SEQUENCE LISTING (31 sequences)

SEQ ID NO: 1 - DNA, 2360 bp, Homo sapiens
  misc_feature (42)...(2225): CDS
ggaagtgcctgcaaccttcgccgctgccttctggttgaagcactatggagggagagaggaagaacaacaacaaacggtggtatttcactcgagaacagctggaaaatagcccatcccgtcgttttggcgtggacccagataaagaactttcttatcgccagcaggcggccaatctgcttcaggacatggggcagcgtcttaacgtctcacaattgactatcaacactgctatagtatacatgcatcgattctacatgattcagtccttcacacggttccctggaaattctgtggctccagcagccttgtttctagcagctaaagtggaggagcagcccaaaaaattggaacatgtcatcaaggtagcacatacttgtctccatcctcaggaatcccttcctgatactagaagtgaggcttatttgcaacaagttcaagatctggtcattttagaaagcataattttgcagactttaggctttgaactaacaattgatcacccacatactcatgtagtaaagtgcactcaacttgttcgagcaagcaaggacttagcacagacttcttacttcatggcaaccaacagcctgcatttgaccacatttagcctgcagtacacacctcctgtggtggcctgtgtctgcattcacctggcttgcaagtggtccaattgggagatcccagtctcaactgacgggaagcactggtgggagtatgttgacgccactgtgaccttggaacttttagatgaactgacacatgagtttctacagattttggagaaaactcccaacaggctcaaacgcatttggaattggagggcatgcgaggctgccaagaaaacaaaagcagatgaccgaggaacagatgaaaagacttcagagcagacaatcctcaatatgatttcccagagctcttcagacacaaccattgcaggtttaatgagcatgtcaacttctaccacaagtgcagtgccttccctgccagtctccgaagagtcatccagcaacttaaccagtgtggagatgttgccgggcaagcgttggctgtcctcccaaccttctttcaaactagaacctactcagggtcatcggactagtgagaatttagcacttacaggagttgatcattccttaccacaggatggttcaaatgcatttatttcccagaagcagaatagtaagagtgtgccatcagctaaagtgtcactgaaagaataccgcgcgaagcatgcagaagaattggctgcccagaagaggcaactggagaacatggaagccaatgtgaagtcacaatatgcatatgctgcccagaatctcctttctcatcatgatagccattcttcagtcattctaaaaatgcccatagagggttcagaaaaccccgagcggccttttctggaaaaggctgacaaaacagctctcaaaatgagaatcccagtggcaggtggagataaagctgcgtcttcaaaaccagaggagataaaaatgcgcataaaagtccatgctgcagctgataagcacaattctgtagaggacagtgttacaaagagccgagagcacaaagaaaagcacaagactcacccatctaatcatcatcatcatcataatcaccactcacacaagcactctcattcccaacttccagttggtactgggaacaaacgtcctggtgatccaaaacatagtagccagacaagcaacttagcacataaaacctatagcttgtctagttctttttcctcttccagttctactcgtaaaaggggaccctctgaagagactggaggggctgtgtttgatcatccagccaagattgccaagagtactaaatcctcttccctaaatttctccttcccttcacttcctacaatgggtcagatgcctgggcatagctcagacacaagtggcctttccttttcacagcccagctgtaaaactcgtgtccctcattcgaaactggataaagggcccactggggccaatggtcacaacacgacccagacaatagactatcaagacactgtgaatatgcttcactccctgctcagtgcccagggtgttcagcccactcagcccactgcatttgaatttgttcgtccttatagtgactatctgaatcctcggtctggtggaatctcctcgagatctggcaatacagacaaaccccggccaccacctctgccatcagaacctcctccaccacttccaccccttcctaagtaaaaaaagaaaaagaagaggagaaaaaaacttctttaaaaaaacacataatttttcttttttttttggggaaaaaaaaattttttttaaaattttttccccaagggacgggggaaaattttatttttaaaatttttt

SEQ ID NO: 2 - DNA, 1791 bp, Homo sapiens
  misc_feature (99)...(1217): CDS
cgcccgccggaggggcctggagtgcggcggcggcgggacccggagcaggagcggcggcagcagcgactgggggcggcggcggcgcgttggaggcggccatggcaaagcagtacgactcggtggagtgccctttttgtgatgaagtttccaaatacgagaagctcgccaagatcggccaaggcaccttcggggaggtgttcaaggccaggcaccgcaagaccggccagaaggtggctctgaagaaggtgctgatggaaaacgagaaggaggggttccccattacagccttgcgggagatcaagatccttcagcttctaaaacacgagaatgtggtcaacttgattgagatttgtcgaaccaaagcttccccctataaccgctgcaagggtagtatatacctggtgttcgacttctgcgagcatgaccttgctgggctgttgagcaatgttttggtcaagttcacgctgtctgagatcaagagggtgatgcagatgctgcttaacggcctctactacatccacagaaacaagatcctgcatagggacatgaaggctgctaatgtgcttatcactcgtgatggggtcctgaagctggcagactttgggctggcccgggccttcagcctggccaagaacagccagcccaaccgctacaccaaccgtgtggtgacactctggtaccggcccccggagctgttgctcggggagcgggactacggcccccccattgacctgtggggtgctgggtgcatcatggcagagatgtggacccgcagccccatcatgcagggcaacacggagcagcaccaactcgccctcatcagtcagctctgcggctccatcacccctgaggtgtggccaaacgtggacaactatgagctgtacgaaaagctggagctggtcaagggccagaagcggaaggtgaaggacaggctgaaggcctatgtgcgtgacccatacgcactggacctcatcgacaagctgctggtgctggaccctgcccagcgcatcgacagcgatgacgccctcaaccacgacttcttctggtccgaccccatgccctccgacctcaagggcatgctctccacccacctgacgtccatgttcgagtacttggcaccaccgcgccggaagggcagccagatcacccagcagtccaccaaccagagtcgcaatcccgccaccaccaaccagacggagtttgagcgcgtcttctgagggccggcgcttgccactagggctcttgtgttttttttcttctgctatgtgacttgcatcgtggagacagggcatttgagtttatatctctcatgcatattttatttaatccccaccctgggctctgggagcagcccgctgagtggactggagtggagcattggctgagagaccaggagggcactggagctgtcttgtccttgctggttttctggatggttcccagagggtttccatggggtaggaggatgggctcgcccaccagtgactttttctaagagctcccggcgtggtggaagaggggacaggtccctcacccacccacaatcctattctcgggctgagaaccctgcgtgaggacagggctcgcctcaggaatgggctgtttttggcctaaccctcagaaacactggggctggcacaaactcttggtttcttcaacaggagaattttactgtgtttcttttggttccattgtttggagacattcctgggcacagtttggtccgttagaattaaaagttgaatttttttttttttttaaaaaaaaaaaaaaaaaaaaaaaaaaaaaaaa

SEQ ID NO: 3 - DNA, 21 bp, Artificial sequence
  RNA molecule with two deoxythymidines at 3′ end
ucccuuccugauacuagaatt

SEQ ID NO: 4 - DNA, 21 bp, Artificial sequence
  RNA molecule with two deoxythymidines at 3′ end
ucccuuccguauacuagaatt

SEQ ID NO: 5 - DNA, 21 bp, Artificial sequence
  RNA molecule with two deoxythymidines at 3′ end
ccaaagcuucccccuauaatt

SEQ ID NO: 6 - DNA, 21 bp, Artificial sequence
  RNA molecule with two deoxythymidines at 3′ end
ccaaagcucuccccuauaatt

SEQ ID NO: 7 - DNA, 3643 bp, Homo sapiens
  misc_feature (46)...(3309): CDS
ggcacgagggaaggcggaggtggagcccgagagcagcagcggaagatgtcggacagcgaggacagcaacttttccgaggaggaggacagcgagcgcagcagtgacggcgaggaggccgaggtagacgaagagcggcggagtgcagcgggcagtgagaaagaagaagagcctgaggacgaagaggaggaggaagaggaggaggaatatgatgaggaagaggaagaagaagatgatgaccgaccccccaagaaaccccgccatggaggcttcattctggacgaggctgatgttgacgatgagtatgaggacgaggaccagtgggaggatggagcagaggacattctagagaaagaagagattgaagcctccaatatcgataatgttgtcctggatgaagatcgttctggggctcgccgcctgcaaaacctctggagggaccagcgagaagaagaactgggcgagtattacatgaagaaatacgccaagtcatctgtgggagagacggtgtatggaggatctgatgagctctcagacgacatcacccagcagcagctgctcccaggagtcaaggatcccaatctgtggactgtcaaatgtaagattggggaggaacgggccacggccatttccttgatgcgcaagttcattgcctaccagttcacagacacgcccctgcagatcaagtcagtagtggcaccagagcatgtgaagggctacatctacgtggaggcctacaagcagacccacgtgaagcaggccattgagggggtgggcaacctgcggcttggctactggaaccagcagatggtgcccatcaaggagatgacagacgtgctcaaagtggtgaaggaggtggccaacctgaaaccaaagtcctgggtccgcctcaagcggggcatctacaaggatgacattgctcaggtggactacgtggagcccagccagaacaccatctccctgaagatgatcccacgcatcgactacgatcgcatcaaggcccgcatgagcttgaaagactggtttgccaaaaggaagaagtttaagcggcctccacagaggctgtttgatgctgagaagatcaggtccctggggggtgatgttgcctctgatggtgacttcctcatctttgaggggaaccgttacagccggaagggctttctgttcaagagcttcgccatgtctgctgtgatcacggagggtgtgaagccaacactctctgagctggaaaagtttgaggaccagccagagggcattgacctggaggtggtgactgagagcacagggaaggagcgggagcacaacttccaacctggggacaacgtggaggtctgtgagggtgagctcatcaacctgcagggcaagatcctcagcgtggatggcaacaagatcaccatcatgcccaagcatgaggacctcaaggacatgttggagttcccagcccaggaacttagaaaatacttcaagatgggggaccacgtgaaggtgattgctggccgattcgagggcgacacaggcctcattgtgcgggtggaggagaatttcgttatcctgttctctgacctcaccatgcatgagctgaaggtgctcccccgggacctgcagctctgctcagagacagcatcaggtgtggatgttgggggccagcatgaatggggcgagctggtgcagctggatccccagactgtgggtgtcatcgtgcgactagaacgggagaccttccaggtgctgaacatgtacgggaaggtggtgactgtcagacatcaggctgtgacccggaagaaggacaaccgctttgctgtggccttggactcagagcagaacaacatccatgtgaaagacatcgttaaggtcattgatggcccccactcaggccgagaaggggagattcgccatctcttccgaagcttcgccttcctacattgcaagaaactggtggagaacgggggcatgtttgtctgcaagacccgccacctggtgctggctgggggctcaaagccccgtgatgtgaccaacttcaccgtgggtggctttgcgcctatgagtccccggatcagcagccccatgcaccccagtgctggaggtcagcgtggcggctttggtagcccaggtggcggcagtggtggcatgagcaggggccggggccggagggacaacgaactcatcggccagaccgtgcgcatctcccaggggccctacaaaggctacatcggtgtggtgaaagatgccacagagtccacggcccgtgtggagctgcactccacctgccagaccatctctgtggaccgtcagcggctcaccacggtgggctcacggcgcccgggcggcatgacctcgacctatgggaggacgcccatgtatggctcccagacgcccatgtatggctctggctcccgaacacccatgtacggctcacagacacccctccaggatggtagccgcaccccacactacggctcacagacgcccctgcatgatggcagccgcactcctgcccagagtggggcctgggaccccaacaaccccaacacgccgtcacgggctgaggaagaatatgagtatgctttcgatgatgagcccaccccgtccccgcaggcctatgggggaacccccaatccccaaacacctggctacccagacccctcgtccccacaggtcaacccacaatacaacccgcagacgccagggacgccggccatgtacaacacagaccagttctctccctatgctgccccctccccacaaggttcctaccagcccagccccagcccccagagctaccaccaggtggcgccaagcccagcaggctaccagaatacccactccccagccagctaccaccctacaccgtcgcccatggcctatcaggctagccccagcccgagccccgttggctacagtcctatgacacctggagctccctcccctggtggctacaacccacacacgccaggctcaggcatcgagcagaactccagcgactgggtaaccactgacattcaggtgaaggtgcgggacacctacctggatacacaggtggtgggacagacaggtgtcatccgcagtgtcacggggggcatgtgctctgtgtacctgaaggacagtgagaaggttgtcagcatttccagtgagcacctggagcctatcacccccaccaagaacaacaaggtgaaagtgatcctgggcgaggatcgggaagccacgggcgtcctactgagcattgatggtgaggatggcattgtccgtatggaccttgatgagcagctcaagatcctcaacctccgcttcctggggaagctcctggaagcctgaagcaggcagggccggtggacttcgtcggatgaagagtgatcctccttccttccctggcccttggctgtgacacaagatcctcctgcagggctaggcggattgttctggatttccttttgtttttccttttagttttccatcttttccctccctggtgctcattggaatctgagtagagtctgggggagggtccccaccttcctgtacctcctccccacagcttgcttttgttgtaccgtctttcaataaaaagaagctgtttggtctaaaaaaaaaaaaaaaaaaaaaaaaaaaaaaaaaaaaaaaaaaaaaaaaaaaaaaaaaaaaaaaaaaa

SEQ ID NO: 8 - DNA, 21 bp, Artificial sequence
  RNA molecule with two deoxythymidines at 3′ end
aactgggcgagtattacatga

SEQ ID NO: 9 - DNA, 21 bp, Artificial sequence
  RNA molecule with two deoxythymidines at 3′ end
aactgggcggatattacatga

SEQ ID NO: 10 - DNA, 21 bp, Artificial sequence
  RNA molecule with two deoxythymidines at 3′ end
tcatgtaatactcgcccagtt

SEQ ID NO: 11 - DNA, 3561 bp, Mus musculus
  misc_feature (35)...(3283): CDS
ggaagcttcaggtggcaagcgatcgctgcggaagatgtcggacagcgaagacagcaacttctccgaggaggaggacagcgagcgcagcagcgaggcggaggaggccgaggttgaggaagaccagcgaagtgctgcaggcagtgaaaaagaggaggagcctgaggaagaggaggaggaagaggaagaatacgatgaggaagaagaggaggaagatgatgataggccccccaagaaaccacgccatgggggcttcattttagatgaggctgatgtagatgatgaatatgaagatgaagaccagtgggaagatggagctgaagatatcctggagaaagaagagattgaagcctccaatattgataacgttgtgctggatgaagaccgctctggggctcgtcgtctgcagaatctctggagggaccagagagaagaagaactgggcgagtattacatgaagaaatatgctaagtcgtctgtgggagagacggtgtatggagggtctgacgagctctcagatgacatcactcagcagcagctgctcccaggagtcaaggaccccaacctgtggactgtcaagtgtaagattggggaggaacgagccacggcaatttccttgatgcggaaattcatcgcttaccagttcacagacacacccctgcagattaagtcagtggttgcccctgagcacgtgaagggctacatctatgtggaggcctacaaacagacccatgtgaagcaggccattgaaggtgtgggcaacctgcggcttggctactggaaccagcagatggtgcctattaaggagatgaccgatgtactcaaggtggtgaaggaggtggccaacctgaagccgaagtcctgggtccgcctcaagcgaggcatctacaaagatgacattgctcaggtggactatgtggagcccagccagaacaccatttccctgaagatgatcccgcgcatcgactacgaccgcatcaaagcccgaatgagcctgaaagactggtttgccaaaaggaagaagttcaagcggcctccacagaggctctttgatgcagagaagatcaggtccttggggggtgatgttgcctctgatggtgacttcctcatctttgaggggaaccgctacagccgaaagggcttcctgttcaagagctttgccatgtctgctgtgattacagaaggtgtgaagcccacactgtcagagctggaaaagtttgaagatcagcctgagggcatcgacctggaggttgtgactgaaagcacagggaaggagcgggaacacaatttccagcctggggacaatgtggaggtgtgcgagggcgagctcatcaacctacagggcaaggtcctcagcgtggatggcaacaagatcactatcatgcccaagcacgaggacctcaaggacatgcttgagttccccgctcaggaacttcggaagtacttcaagatgggggaccacgtaaaagtgattgctggccgatttgagggtgacacaggtctcattgtgcgggtggaagagaactttgttattctcttttcagacctcaccatgcatgagttgaaagtgcttccccgagacctgcagctctgctcagagacagcctcaggcgtggatgttgggggccagcatgagtggggtgagctggtgcagctggatccccgtactgtgggtgtcatcgtgcggctagagcgagaaaccttccaggttctcaacatgcacgggaaggtggtgaccgttaggcaccaggccgtgacgcagaagaaggacaaccgcttcgctgtggctctggactcggatcagaacaacatccatgtgaaggacattgtcaaggtcatcgatggccctcactcaggccgagagggtgagatccgccatctgtaccgcagctttgccttcctgcactgcaagaagctggtggagaacggcggcatgtttgtctgtaaggcacggcacctggtgttggctgggggctcaaagccccgggatgtgaccaatttaactgttggtggcttcactcctatgagcccccggatcagcagccctatgcaccccagtgctgaaggtcagcatggtggctttggaagcccaggtggcatgagcaggggtcgagggagaagagacaatgaactcataggccagactgtgcgtatctcccagggaccctacaaaggctacattggtgtggtgaaggatgccacagagtccacggccagagtagaactgcattctacctgccagaccatctctgtggatcgccagcggctcaccacggtcgactcccagcgtccaggtggcatgacctctacatatggacggactcccatgtatggctctcagacacccatgtatggctctggctcccgaacacctatgtatggctcccagacacctttgcaggatggcagccgcactccacattatggctcacagacacccctgcatgatggtagccgcactcctgctcagagtggggcctgggaccccaacaacccaaacacgccttccagggctgaggaggaatacgaatatgcctttgacgacgaacccaccccatccccacaagcatatgggggaaccccgaatccccaaacacccggctacccagacccatcatccccccaggtcaatccacagtacaatccacagacaccagggacgccagccatgtacaacacagaccagttctccccctacgctgccccctctccacaggggtcttaccagcccagccccagcccccagagctaccaccaagtggcaccaagtccagcaggctatcagaacacccattccccagccagctatcacccgacaccgtcacctatggcctatcaggctagtcccagcccaagccctgttggctacagtccaatgacccctggagcaccttctcctggaggttacaatccccacacaccgggctcaggcattgagcagaactctagcgactgggtgaccactgatatccaggtgaaggttcgagacacctatctggacacacaaatagtggggcagacaggcgtcatccgcagtgtcacgggaggcatgtgctccgtgtacctgaaggacagtgagaaggtggtcagcatctccagtgaacatctggagcccatcacgcccaccaagaacaacaaggtgaaggtgattctgggtgaggaccgagaggctaccggggtcctactgagcattgatggtgaggatggcatcatccgcatggacctagaggaccagcagatcaagattctaaacctccgcttcctagggaagctcctggaagcctgacgcccaagtgtgcagtgcagacatggagccagcagcccctctggccttgtttggcctagcatggaaccctgagtctggagcaagccagggcaatcactctgtatttccttttcctccttccctgatactctaggagtcagagcagagtctgaggaatgttcctgccttcttctgcagccccagcttgactttgtgttcaggtctttcaataaagagaagctgtttgaaaaaaaaaaaaaaaaaaaaaaaaaaaaaaaaaaaaaaaaaaaaaaaaaaaa

SEQ ID NO: 12 - DNA, 1499 bp, Homo sapiens
  misc_feature (68)...(421): CDS
tatttacttcctgcgggtgcacaggctgtggtcgtctatctccctgttgttcttcccatcggcgaagatggccctggagacggtgccgaaggacctgcggcatctgcgggcctgtttgctgtgttcgctggtcaagactatagaccagtttgaatatgatggttgtgacaattgtgatgcatatctacaaatgaagggtaaccgagagatggtatatgactgcactagctcttcctttgatggaatcattgcgatgatgagtccagaggacagctgggtctccaagtggcagcgagtcagtaactttaagccaggtgtatatgcggtgtcagtcactggtcgcctgccccaaggaatcgtgcgggagctgaaaagtcgaggagtggcctacaaatccagagacacagctataaagacctagcaagatgcaaggctgccagcatctttgctctccacctcctgcctctgcttatttcttgttctggaactaaatgaacagaacttcaaatacttcctaccctccaattcagactcagctgactgttgagagagcagcacatcattttatcattttatcttctttggactacaggtggggtgggagggatttgggttggtggattaacagatggaattgaggagagagtaggatgctgattttcctacccgtggcccaggtctgtgccttccccatgccaaggactctaggtcaaatgtcaataaatatgaacctcgagaaagttctgaaggccatgacacctgccttgcctccctcttccattctcttaggcacagtaatagcttatttgccctataagaaccttcccagagcagcagaggcccttctactccctcttgactgtctcagcctctgggattgcagcctttgtagtgtgcttccttgcttcctatcagagggtgctgatccagaggctcagtaaccccatcaacttggtggccctggtgtctcacacttgtatccttctgccctcgagacctggcacagcagtatcccttgaagaaatcctgaggctttgtagagtgctccttgaccatgtttaataattcttccctcccctgcttgtctattttcttctcttcacggctcttcctataccttaggccagtctcaagcactcactggagacccttgggccttgggcgaccattgagtcctagtctcccttgtttgtgcccctgtaggaggtaggtccttttctcctccggcctagtaggggaccttgggtaacatcccatttttcggccaaggtgagttgttttaggataaaaaaatttaccacaaattctcatttaaatttccacagaaatcctgttcgtatccccattttgatttccctaagttccttgttctccctctaaaaagagaatgattgcaccctgcctgtttacctcaggattgttgtgattgtagaaacgaagctatgtgaaaattatataagtattataaaggtgaaatacttttgctctcaaaaaaaaaa

SEQ ID NO: 13 - DNA, 693 bp, Mus musculus
  misc_feature (36)...(389): CDS
cggctggaggaacctgtgttgtcgtatcggtgaggatggccctggagacggtaccaaaggacctgcggcatctgcgggcttgtttgctgtgctcgttagtcaagactatagaccagttcgaatatgatgggtgtgacaattgcgatgcatacctacaaatgaagggcaacagagagatggtttatgactgcaccagctcttcatttgatggaatcattgcgatgatgagtccagaggacagctgggtctccaagtggcagcgagtcagtaactttaagccaggtgtatatgctgtgtccgtcactggtcgcctgccccaaggaatcgtgcgggagctgaaaagtcgaggagtggcctacaaatccagagacacagcaataaagacctagctaggtgcaggccatagcatcttgttcttcacttctgtacttgttttcttgtggaactaaaccgtgggaccccaaagactccccgccctccatctccgaatcagcagactccacgaaagcagcacatcggccttaagtcatctcactttctctgcccactggtgaagtgggagtgtctgggcggattctcagaggctgtgtcaacagaggaagatgctgatttcccacctgtgttcagggttttgcctttgtctatgggcaggactttggttcaaataccaataaagcgagcctttggaacata

SEQ ID NO: 14 - DNA, 5923 bp, Homo sapiens
  misc_feature (185)...(5365): CDS
ttaacgcagcagcggcgacggtggtggtggtggtggcggcggctgcggaggccttgcggtgggtccggactatccttttgcagtctcttgatcggcgactccattttcttcggtgggagcttagcgtagcggagaagcagcggccttcaagcagtcttcaaggaggctgtgagaagcagcaacaatgtctgattttgtggaaagtgaggccgaagagtcagaagaagaatacaatcatgaaggcgaggtggtacctagagtcaccaagaagtttgtggaagaggaggatgatgatgaggaggaggaagaggagaatctagatgatcaagatgagcgaggcaacctcaaagactttatcaatgacgatgatgatgaagaagaaggggaggaggatgagggcagcgactctggggactcagaagacgatgttggccacaagaagagaaaacgcccatcttttgatgaccgcctggaagatgatgattttgacctcattgaggagaatttgggtgttaaagtcaaacgaggacaaaaataccgacgtgtcaaaaaaatgtctgatgatgatgaagatgatgaagaggaatatggcaaagaggaacacgagaaggaggccattgcaggggaaatcttccaagatgaagaaggggaagaaggccaggaggctgtagaggcccccatggcccctcctgacgaggaggaggaagatgatgaggagtcagacattgacgatttcattgtggatgatgatggacagcctctgaaaaaacccaaatggcgaaaaaagctccctggatatacagatgcagccctgcaggaagcccaggagatcttcggtgtggactttgactatgatgaatttgagaaatacaatgaatacgatgaagaactggaagaagactatgagtacgaggatgatgagactgaaggtgagatccgagtgcgccccaagaagaccaccaagaaacgtgtgagtcgcaggagcatctttgagatgtacgagcctagtgagctggagagcagccacctcacagaccaggacaatgaaatccgagccactgacttgcctgagaggttccagctccgctccatcccagtcaaggcagctgaggatgatgagctagaagaggaagctgactggatctatagaaatgccttcgcaacaccgaccatctccctccaggacagctgtgattacctagaccgtgggcagccaactagtagcttcagtcgtaaagggcccagcacagttcagaagatcaaagaggccctgggcttcatgcggaatcagcattttgaggtgcctttcattgccttctaccgaaaggagtatgtggaacctgagctgcacatcaatgacctgtggagggtgtggcagtgggatgagaagtggactcagctgagaatccgcaaagagaacctaacacggctgtttgagaagatgcaggcttatcagtatgagcaaatctctgctgaccctgacaaacctcttgctgacggcatccgggctctagatactactgacatggagaggctcaaagatgttcagtcaatggatgaactgaaagacgtctacaaccatttcttactttactacgggcgtgacatccccaagatgcagaatgctgccaaggctagccggaagaagctgaaacgcataaaggaggatggcgatgaagaaggtgaaggtgaggaggctgaagatgaggaacagaggggaccagagctcaagcaagcctcccgccgagacatgtataccatctgccagagtgccgggctagatggcctggccaaaaagtttggcctcacacctgagcagtttggggagaacctccgtgacagctaccagcgccacgagacagagcagttccctgcagagcctttggagctggccaaagattacgtgtgcagccagttccctacaccagaagctgtgcttgaaggtgcccgatacatggttgcactacagattgccagggagccccttgtcagacaggtgctgagacaagcctttcaggagagagccaagctaaacataaccccaaccaagaaaggtagaaaggatgtggatgaggcccactacgcttactcttttaagtacttaaagaacaagcctgttaaagagctgagagacgaccagttcctcaagataggcctggctgaggatgaagggctgctcaccattgacatcagcatagatatgaagggagtggaagggtatggtaatgaccagacatattttgaggagatcaagcagttttactaccgggatgagttcagccaccaagtacaggaatggaaccggcagcgcactatggccattgagcgagctctgcagcagttcctctacgtgcaaatggccaaagaacttaagaacaagctgctagctgaggccagggagagtgttgtgaaggcctgtagccgaaagctctacaactggttgagagtggcaccctaccgtccagatcagcaggtagaggaggacgacgactttatggatgagaaccaggggaagggcatccgagtcctaggcattgctttctcctctgccagagatcaccctgtgttctgtgccctagtcaatggtgaaggagaagtgactgatttcctccgcctgccccattttaccaagcggcgaactgcatggagagaagaggagcgggaaaagaaggctcaagatattgaaaccctaaagaagtttcttgtgaataagaagcctcatgtggtcactatcgcaggagagaacagggatgcccagatgctgactgaggatgtgaagcggatagtgcatgagctggaccaaggccaacagctgtcttctattggggtggagctggttgacaatgagctggccattctgtacatgaacagcaagaaatcagaggcagagttccgggattatccaccagttttgagacaagctgtctctctagcccggcgtatccaagaccctctgatagaatttgcccaggtgtgcagctcagatgaagacatcctgtgtctcaagtttcatcccttgcaggagcatgttgtgaaagaggagctgcttaacgccttgtactgtgaattcatcaaccgggtcaatgaggtcggggtggatgtcaaccgtgccattgcccatccttacagccaggccttgatccagtatgtctgtggcctgggaccccgaaaagggacccacctgctgaagattttgaaacagaacaacactcgactcgagagccggactcagctggtcaccatgtgtcatatgggtcccaaagtcttcatgaattgtgctggcttcctcaagatcgacactgcttctctgggggacagcactgactcatacattgaagttcttgatggctctcgagtccaccctgaaacgtacgagtgggccaggaagatggcagtggatgccttagaatacgatgagtctgcagaagatgccaatcctgctggagcccttgaggaaatcttggaaaacccagagcggctgaaggatttagatcttgatgcctttgcagaagaactagagagacagggctatggtgacaagcacatcacactgtatgacatccgagcagagctcagttgtaggtataaggacctccggacagcctaccgctctcctaacacagaggagatcttcaatatgttaaccaaagagacgccagagaccttctacattggaaagcttatcatctgcaatgttaccggcattgctcacaggcgcccacagggtgagagctatgaccaggccatccgaaatgatgagacagggctgtggcagtgccccttctgtcaacaagacaacttccctgagctaagcgaggtctggaaccattttgacagtggatcatgccctggtcaggccattggtgtgaaaacacggctagacaatggcgtcaccggcttcatccctaccaaattcctcagtgacaaggtggtgaagaggccagaggagcgagtgaaggtggggatgactgttcactgccggatcatgaagatcgatattgaaaagttcagtgcagacctgacctgccggacctcagacctcatggacaggaacaatgagtggaaactgcccaaggacacctactatgactttgatgcggaagctgcagaccacaagcaggaagaggatatgaaacgaaagcagcagcggaccacatacatcaagcgagtgattgcacacccgtccttccataacataaatttcaagcaagcagaaaagatgatggagaccatggaccaaggggatgttatcattcggcccagcagtaaaggtgaaaaccatctgacggtgacctggaaagtcagtgctggcatctaccagcacgtggacgtgcgggaagagggcaaggagaacgccttcagcctgggagccactctttggatcaacagtgaggaatttgaagacttggatgagattgttgcccgctatgtccagcccatggcttcctttgctcgagaccttctgaaccacaaatattaccaggactgtagtggtggggaccgaaagaaattagaggagctgctcatcaaaactaagaaggagaagcccaccttcatcccttacttcatctgtgcctgcaaggagctgcccggcaagttcctcctgggataccagccccggggtaaacccaggatagaatatgtaacagtgactcctgaaggattccggtaccggggccagattttcccaactgtgaatggactcttcaggtggtttaaggatcactatcaggatcctgtaccaggcatcactcccagtagcagtaacagaacacggacccctgcttctatcaatgctaccccagccaacatcaatcttgcagatctcacacgggctgtgaatgccttgcctcagaacatgacttcacagatgttcagtgccattgctgctgtgacaggccaggggcagaaccccaatgctaccccagcccagtgggcatccagccagtatggctacggtggcagcggaggaggcagcagcgcctaccatgtgttcccaacaccagcccagcagccagtggccacaccactaatgaccccaagctactcctatactaccccgagccagcccatcaccaccccacaataccaccagctccaggccagcaccaccccacagtctacacaggcccagccccagccttcctccagctcccgacaacggcaacagcagccaaagtccaacagccatgcagctatcgactggggcaagatggccgagcagtggctacaggaaaaggaggcagaacgacggaaacagaaacagcggctgaccccacgaccctctcccagccccatgatcgagagcacccccatgtccattgctggcgatgcgaccccactcctggacgagatggatcggtagggggcctgttcctcggactctggttacctctgaagctcccaagaggcctggccacttatggcctcactccctgcccctcttttttttttgtccataaagtggcgtgaaatgacgttctttttagtggtcagcctggctggatggtggccttgtgagctggtgtgcaccgagcagcagctgttccactccgggcttgtgctaaccacctgttctggggccaggctgggagagcagtatggcagggaggagagcaaaggtggagcagtctgtaatgtctctggaagccctcacctgagacaggccatcatgaggcatgcagctcaattgtctatgtccccagcctgtgtggggacaccgatgctatttattcagtttggggcaggagggaaagtagctctgatcctgatggcaagaccttggctgtcaaagcaggactgtgagggcatgtggagcagtgctgagctgccagcccctgcctgcctgcctgcctgccttccctcatcacctaacctcacctgcttctcgcctgtcatttgaataaacagtgtttgtacagag

SEQ ID NO: 15 - DNA, 20 bp, Artificial sequence
  RNA molecule with two deoxythymidines at 3′ end
ggaggcggccatggcaaagc

SEQ ID NO: 16 - DNA, 20 bp, Artificial sequence
  Synthetic construct
tgaagcactatggagggaga

SEQ ID NO: 17 - DNA, 21 bp, Artificial sequence
  RNA molecule with two deoxythymidines at 3′ end
uuggucuccuugaugcuuutt

SEQ ID NO: 18 - DNA, 21 bp, Artificial sequence
  RNA molecule with two deoxythymidines at 3′ end
gaaacguagacagcgcagatt

SEQ ID NO: 19 - DNA, 21 bp, Artificial sequence
  RNA molecule with two deoxythymidines at 3′ end
gcagcacgacuucuucaagtt

SEQ ID NO: 20 - DNA, 21 bp, Artificial sequence
  RNA molecule with two deoxythymidines at 3′ end
gugggagcgcgugaugaactt

SEQ ID NO: 21 - DNA, 21 bp, Artificial sequence
  RNA molecule with two deoxythymidines at 3′ end
uucuaguaucaggaagggatt

SEQ ID NO: 22 - DNA, 21 bp, Artificial sequence
  RNA molecule with two deoxythymidines at 3′ end
uucuaguauacggaagggatt

SEQ ID NO: 23 - DNA, 21 bp, Artificial sequence
  RNA molecule with two deoxythymidines at 3′ end
uuauagggggaagcuuuggtt

SEQ ID NO: 24 - DNA, 21 bp, Artificial sequence
  RNA molecule with two deoxythymidines at 3′ end
uuauaggggagagcuuuggtt

SEQ ID NO: 25 - DNA, 21 bp, Artificial sequence
  RNA molecule with two deoxythymidines at 3′ end
ucauguaauacucgcccagtt

SEQ ID NO: 26 - DNA, 21 bp, Artificial sequence
  RNA molecule with two deoxythymidines at 3′ end
ucauguaauauccgcccagtt

SEQ ID NO: 27 - DNA, 21 bp, Artificial sequence
  RNA molecule with two deoxythymidines at 3′ end
cugggcgaguauuacaugatt

SEQ ID NO: 28 - DNA, 21 bp, Artificial sequence
  RNA molecule with two deoxythymidines at 3′ end
aaagcaucaaggagaccaatt

SEQ ID NO: 29 - DNA, 21 bp, Artificial sequence
  RNA molecule with two deoxythymidines at 3′ end
ucugcgcugucuacguuuctt

SEQ ID NO: 30 - DNA, 21 bp, Artificial sequence
  RNA molecule with two deoxythymidines at 3′ end
cuugaagaagucgugcugctt

SEQ ID NO: 31 - DNA, 21 bp, Artificial sequence
  RNA molecule with two deoxythymidines at 3′ end
guucaucacgcgcucccactt

(19) United States
(12) Patent Application Publication
(10) Pub. No.: US 2004/0152651 A1
(43) Pub. Date: Aug. 5, 2004

(54) Regulation of transcription elongation factors

(76) Inventor: Tariq M. Rana, Shrewsbury, MA (US)

Correspondence Address: LAHIVE & COCKFIELD, LLP., 28 STATE STREET, BOSTON, MA, 02109, US

(21) Appl. No.: 10/635,854

(22) Filed: Aug. 5, 2003

Related U.S. Application Data
(60) Provisional application No. 60/423,198, filed on Nov. 1, 2002.
(60) Provisional application No. 60/439,301, filed on Jan. 9, 2003.
(60) Provisional application No. 60/433,097, filed on Dec. 13, 2002.

(51) Int. Cl. A61K 48/00; C07H 21/04; C07H 21/02

(52) U.S. Cl. 514/44; 536/23.1

(57) ABSTRACT

The present invention relates to agents, including siRNA and shRNA molecules, small molecules, antisense strands, and ribozymes that are targeted to transcription elongation factors (TEFs), including CDK9 and CycT1, subunits of P-TEFb, Spt4 and Spt5, subunits of DSIF (DRB Sensitivity-Inducing Factor (DSIF)), and Spt6. The present invention also relates to methods for treating disorders associated with aberrant or unwanted TEF expression or activity, including HIV and disorders characterized by unwanted or aberrant cellular proliferation or differentiation, such as cancer.

### RELATED APPLICATIONS

This application claims the benefit of U.S. Provisional patent application Serial No. 60/423,198, entitled “RNA Interference of Positive Elongation Factors”, filed Nov. 1, 2002 (pending); U.S. Provisional patent application Serial No. 60/433,097, entitled “Regulation of Transcription Elongation Factors”, filed Dec. 13, 2002 (pending); and U.S. Provisional patent application Serial No. 60/439,301, entitled “Inhibition of Positive Elongation Factors”, filed Jan. 9, 2003 (pending). The entire content of the above-reference patent applications is hereby incorporated by this reference.

### GOVERNMENT RIGHTS

This invention was made with Government support under Grant Nos. A141404, A145466 and A143198 awarded by the National Institutes of Health. The Government has certain rights in the invention.

### BACKGROUND OF THE INVENTION

Regulation of mRNA transcription plays a central role in mammalian cell growth and development. Eukaryotic mRNA synthesis is catalyzed by multisubunit RNA polymerase II and proceeds through multiple stages referred to as preinitiation, initiation, elongation, and termination. Many genes in human and other eukaryotic cells, as well as in viruses, are specifically regulated at the level of transcription elongation. During elongation, RNA polymerase II (RNA pol II) can pause, get arrested, pass through terminator sequences, or terminate transcription. Shortly after initiation, RNA pol II faces a barrier of negative transcription elongation factors (N-TEFs) and enters abortive elongation (Price, Mol. Cell. Biol. 20:2629-2634 (2000)). Positive transcription elongation factors (P-TEFs) lower the barrier of N-TEFs and help RNA pol II escape from this transition phase, thus preventing possible premature termination of transcription (Price (2000), supra).

Positive transcription elongation factor complex b (P-TEFb), which is composed of two subunits, CDK9 and cyclin T1 (CycT1) (Garber et al., Genes & Dev., 12:3512-3527 (1998)), allows the transition to productive elongation, producing longer mRNA transcripts (Price (2000), supra). Two negative transcription elongation factors, DSIF (DRB sensitivity-inducting factor; DRB is 5, 6-dichloro-1-β-D-ribofuranosylbenzimidazole) and NELF (negative elongation factor), have been identified and characterized (Wada et al., Genes Dev. 12:343-56 (1998); Yamaguchi et al., Cell 97:41-51 (1999)). DSIF is composed of at least two subunits, one 14-kDa and one 160-kDa, which are homologs of the Saccharomyces cerevisiae transcription factors Spt5 and Spt4, respectively (Hartzog et al., Genes Dev. 12:357-369 (1998)). NELF is composed of five polypeptides, named as NELF-A to -E, and contains a subunit identical to RD, a putatitive RNA-binding protein (containing arginine-aspartic acid (RD) dipeptide repeats) of unknown function. DSIF and NELF function cooperatively and strongly repress RNA pol II elongation (Yamaguchi et al., supra). In the absence of P-TEFb, DSIF plays the role of a negative regulator in transcription (Wada et al., EMBO J. 17:7395-7403 (1998)). DSIF subunit Spt5 also has a positive elongation activity in Tat transactivation (Wu-Baer et al., J. Mol. Biol. 277:179-197 (1998); Kim et al., Mol. Cell. Biol. 19:5960-598 (1999)). Another transcription elongation factor, Spt6, has been identified which is functionally related to Spt5; Spt5 and Spt6 have been shown to colocalize at regions of active transcription as well as at certain stress response genes induced by heat shock (Kaplan et al., Genes Dev. 14:2623-2634 (2000); Andrulis et al., Genes Dev. 14: 2635-2649 (2000)).

Among the genes regulated in this manner are several protooncogenes (c-myc, c-myb, c-fos); c-fms, the gene encoding macrophage colony stimulating factor 1 (CSF-1) receptor; the gene encoding adenosine dearninase; a collection of stress response genes including hsp70 and genes involved in replication and pathogenesis of HIV-1 and HIV-2.

One elegant example of transcription elongation control is the mechanism of HIV-1 gene expression (reviewed in: Cullen 1998 Cell 93:685-92; Emerman and Malin 1998 Science 280:1880-4; Jeang et al. 1999 J Biol Chem 274:28837-40; Jones 1997 Genes Dev 11:2593-2599; Karm 1999 J Mol Biol. 293:235-254; Taube et al. 1999 Virology 264:245-253). HIV-1 encodes a small regulatory protein, Tat, which is required for efficient transcription of viral genes. Tat enhances the processivity of RNA pol II elongation complexes that initiate transcription in the HIV long terminal repeat (LTR) region. During elongation, Tat binds to a highly structured RNA element, transactivation responsive (TAR) RNA, which is located at the 5′-end of nascent viral transcripts (Rana and Jeang 1999 Arch Biochem Biophys 365:175-185). Through these interactions with TAR RNA, Tat controls an early transcription elongation step that is sensitive to protein kinase inhibitors and requires the carboxyl-terminal domain (CTD) of the large subunit of RNA pol II (Jones 1997 Genes De.v 11:2593-2599). The HIV-1 transcriptional activation mechanism requires Tat interactions with the human Cyclin T1 (hCycT1) subunit of P-TEFb that recruits the kinase complex to the pol II elongation machinery (Bieniasz et al. 1998 EMBO J 17:7056-65; Herrmann and Rice 1995 J. Virol. 69:1612-1620; Herrmann and Rice 1993 Virology 197:601-608; Isel and Karn 1999 J. Mol Biol. 290:929-941; Jones 1997 Genes Dev. 11:2593-2599; Mancebo et al. 1997 Genes Dev 11:2633-2644; Taube et al. 1999 Virology 264: 245-253; Wei et al. 1998 Cell 92:451-62; Yang et al. 1997 Proc Natl Acad Sci USA 94:12331-12336; Zhu et al. 1997 Genes Dev. 11:2622-32)

Recruitment of P-TEFb to TAR RNA is proposed to be both necessary and sufficient to activate transcription elongation from the HIV-1 LTR promoter (Bieniasz et al. 1999 Proc Natl Acad Sci USA 96:7791-7796). Neither hCycT1 nor the P-TEFb complex bind TAR RNA in the absence of Tat, signifying that binding to RNA is highly co-operative for both Tat and P-TEFb (Garber et al. 1998 Genes Dev. 12:3512-3527; Richter et al. 2002 Biochemistry 41:6391-6397; Wei et al. 1998 Cell 92:451-62). Mutagenesis studies have shown that the hCycT1 sequence containing amino acids 1-272 were sufficient to form complexes with Tat-TAR (Bieniasz et al. 1998 EMBO J. 17:7056-65; Fujinaga et al. 1998 J Virol 72:7154-7159; Garber et al. 1998 Genes Dev. 12:3512-3527; Ivanov et al. 1999 J Mol Biol 2888:41-56; Wimmer et al. 1999 Virology 255:182-189; Zhou et al. 1998 EMBO J 17:3681-3691). Human Cyclin T1 residues 250-262 represent the Tat-TAR RNA recognition motif (TRM) (Garber et al. 1998 Genes Dev. 12:3512-35273), which is required but not sufficient to form the hCycT1-Tat-TAR RNA ternary complex, and N-terminal residues in the cyclin box are also necessary for complex formation. Specifically, residues 252-260 of hCycT1 interact with one side of the TAR RNA loop, enhancing the interaction between Tat residue K50 and the other side of the loop (Richter et al. 2002 Proc Natl Acad Sci USA 99:7928-2933). Thus, it has become clear that TAR RNA provides a scaffold for two protein partners to bind and assemble a regulatory switch in HIV replication.

The pol II CTD, and Spt5 are also intimately connected to this regulation of HIV gene expression by Tat and P-TEFb. Human Spt5 and its binding partner hSpt4 comprise the transcription elongation regulatory factor DSIF (DRB sensitivity inducing factor) (Wada et al. 1998 Genes Dev 12:343-356). DSIF binds to pol II and, in concert with NELF (negative elongation factor), represses elongation at promoter-proximal positions in the transcription unit (Renner et al. 2001 J Biol Chem 276:42601-42609; Yamaguchi et al. 1999 Cell 97:41-51). Escape from the repressive effect of DSIF/NELF requires the action of P-TEFb, which phosphorylates both the pol II CTD and the Spt5 subunit of DSIF (Ivanov et al. 1999 J Mol Biol 288:41-56; Kim and Sharp 2001 J Biol Chem 276:12317-12323; Ping and Rana 2001 J Biol Chem 276:12951-12958; Wada et al. 1998 EMBO J. 17:7395-7403). HIV has usurped this mechanism for transcribing its own genome (see model; FIG. 1A). During HIV transcription, P-TEFb, which is initially found as a component of the pol II preinitiation complex (PIC), travels with the transcription elongation complex (TEC) as it moves along the HIV transcription unit (Ping and Rana 1999 J Biol Chem 274:7399-7404). In contrast, DSIF and NELF are not present in the PIC, but associate with the TEC at promoter proximal positions and then travel with the TECs down the template (Ping and Rana 2001 J Biol Chem 276:12951-12958).

In mice, the CDK9/PITALRE protein appears to be expressed predominantly in tissues that are terminally differentiated such as the developing brain and the dorsal root ganglia, areas of skeletal muscle, cardiac muscle, and the lining of the developing intestinal epithelium. Analysis of expression pattern of the murine CDK9/PITALRE protein in adult mouse tissues by immunoblotting indicates that murine CDK9/PITALRE expression is ubiquitous, however, steady-state protein levels are markedly higher in the brain, liver, lung, spleen and kidney. Kinase activity of CDK9/PITALRE kinase also detected in the same adult tissues and was highest in the mouse brain, liver, spleen and lung. Only minimal kinase activity was found in the heart, muscle, and kidney. Bagella et al., J. Cell. Physiol. 177:206-213 (1998). In adult human tissues, CycT1 expression is ubiquitous, with higher immunoreactivity in some tissues of mesenchymal origin, such as cardiovascular and connective tissues, skeletal muscle cells, myocardial cells, adipocytes, chondrocytes and endothelial cells, blood, and lymphoid tissues. Astrocytes, oligodendroglial, and microglial cells of the brain tissue also had a high level expression of Cyclin T1 while endocrine and reproductive systems showed low Cyclin T1 expression. (De Luca et al. (2001), J. Histochem. Cytochem., 49(6), 685-92).

### SUMMARY OF THE INVENTION

The present invention is based in part on the discovery that the expression of factors involved in transcriptional regulation, e.g. TEFs such as P-TEFb (CDK9/CycT1), DSIF (Spt4/Spt5) and Spt6, can be specifically reduced without fatal consequences to a cell, such as a mammalian cell. Thus, CDK9, CycT1, Spt4, Spt5, and/or Spt6 are targets for the treatment of disorders characterized by unwanted or aberrant CDK9, CycT1, Spt4, Spt5 and/or Spt6 activity, including viral disorders such as HIV/AIDS, or disorders characterized by unwanted or aberrant cellular proliferation or differentiation, such as cancer, wherein it is desirable to reduce or eliminate TEF activity in a cell, in some cases, without killing the cell. The present invention provides a number of methods for reducing TEF activity by specifically targeting one or more TEFs, e.g., CDK9, CycT1, Spt4, Spt5, and/or Spt6, including siRNA, antisense, ribozymes, and small molecules, which are useful both experimentally and therapeutically.

Other features and advantages of the invention will be apparent from the detailed description, drawings, and from the claims.

### DESCRIPTION OF DRAWINGS

FIG. 1 is a schematic of a theoretical model for HIV Tat transactivation involving the human P-TEFb (CyclinT1/CDK9) complex.

FIG. 2A is a Western blot of specific hCycT1 and CDK9 RNAi activities in HeLa cells transfected with double-stranded (ds) siRNAs targeting RFP (control, lanes 1-7).

FIG. 2B is a Western blot of specific hCycT1 and CDK9 RNAi activities in HeLa cells transfected with double-stranded (ds) siRNAs targeting hCycT1 (lanes 8-14).

FIG. 2C is a Western blot of specific hCycT1 and CDK9 RNAi activities in HeLa cells transfected with double-stranded (ds) siRNAs targeting CDK9 (lanes 15-21).

FIG. 2D is a Western blot of specific hCycT1 and CDK9 RNAi activities in HeLa cells transfected with double-stranded (ds) mutant siRNAs targeting hCycT1 [2 nucleotide mismatches, lanes 22-28].

FIG. 2E is a Western blot of specific hCycT1 and CDK9 RNAi activities in HeLa cells transfected with double-stranded (ds) siRNAs targeting CDK9 [2 nucleotide mismatches, lanes 29-35]).

FIG. 3A is a photograph of a gel showing specific hCycT1 and CDK9 RNAi activities by RT-PCR at the time points indicated in HeLa cells transfected with hCycT1 ds siRNA (lanes 1-7).

FIG. 3B is a photograph of a gel showing specific hCycT1 and CDK9 RNAi activities by RT-PCR at the time points indicated in HeLa cells transfected with CDK9 ds siRNA (lanes 8-14).

FIG. 4 is a series of photomicrographs depicting the results of analysis of cell viability by in vivo fluorescence analysis of HeLa cells cotransfected by Lipofectamine™ with pEGFP-C1 reporter (GFP) plasmid and four siRNA duplexes, including a control duplex targeting RFP (panels a and e) and three duplexes targeting hCycT1 (panels b and f), CDK9 (panels c and g), and CDK7 (panels d and h). Reporter gene expression was monitored at 50 hours post transfection by fluorescence imaging in living cells (upper panels). Cellular shape and density were recorded by phase contrast microscopy (lower panels).

FIG. 5 is a line graph depicting the results of analysis of cell viability by counting, at the indicated time points, trypan blue-stained HeLa cells cotransfected by Lipofectamine™ with pEGFP-C1 reporter (GFP) plasmid and four siRNA duplexes, including a control unrelated duplex (circles) and three duplexes targeting hCycT1 (diamonds), CDK9 (squares), and CDK7 (triangles).

FIG. 6 is a Western blot of hCycT1 and CDK9 RNAi activities in Magi cells co-transfected with pTat-RFP plasmid and various siRNAs as indicated using antibodies against hCycT1 (upper panel) and CDK9 (lower panel).

FIG. 7 is a photomicrograph of β-galactosidase stained Magi cells, either untransfected (panels a, c, e, and g) or transfected (panels b, d, f and h) with pTat-RFP in the presence of mismatched hCycT1 siRNA (mm) (panels b and f) or hCycT1 ds siRNA (panels d and h).

FIG. 8 is a bar graph depicting the results of P-TEFb silencing by RNAi on Tat transactivation in Magi cells cotransfected with pTat-RFP plasmid and ds siRNAs targeting hCycT1 and CDK9 (bars 4 and 5), with antisense (as) RNA strands (bars 2 and 3), or mutant (mm) siRNAs (bars 6 and 7). Green fluorescent protein (GFP) ds siRNA was used as an unrelated control siRNA (bar 8), while Tat ds siRNA, targeting the mRNA encoding Tat sequence, was used as a positive control (bar 9). Means±SD of two experiments are shown.

FIG. 9 is a bar graph depicting the effect on [symbol] -galactosidase activity in HeLa-CD4-LTR/β-galactosidase (Magi) cells transfected with homologous (ds, bars 3 and 4) and mismatched (mm, bars 5 and 6) siRNAs directed against CycT1 or CDK9, mock transfected without siRNA (bar 2) or transfected with an unrelated ds siRNA against the RFP sequence (bar 7).

FIG. 10 presents an evaluation of P-TEFb kinase activity in P-TEFb knockdown cells. (A) Experimental procedure for assaying P-TEFb kinase activity from cells with or without hCycT1 siRNA treatment. See Material and Methods for details. (B) Kinase activity of P-TEFb. P-TEFb and its associated factors were affinity purified (anti-CDK9 Immunoprecipitation) from HeLa cell extract and treated (lanes 1-7) or not treated (lanes 8-14) with RNase A as outlined in (A). Kinase assays were performed on anti-CDK9 immunoprecipitates at 37° C. for 1 h in 20 mM Tris-HCl (pH 8.0), 5 mM MgCl₂, 60 mM NaCl and 10 μM ATP and [γ-³²P]-ATP in a total volume of 45 ml. The reaction was terminated by the addition of 15 ml of 4×Laemmli sample buffer. Phosphorylated protein was visualized by autoradiography after electrophoresis in a 10% SDS-polyacylamide gel (upper panel). Amount of hCycT1 and CDK9 proteins in the immunoprecipitates were eluted with SDS, resolved by 10% SDS-PAGE, and stained with Bio-Rad silver stain plus kit (bottom panel). The specificity of the protein bands was confirmed by immunoblotting with anti-hCycT1 or anti-CDK9 antibodies.

FIG. 11 is a model for how siRNA-mediated P-TEFb silencing modulates HIV-1 transcription without causing major lethal effect to the host cells.

FIG. 12 is a chart showing the results of genome-wide analysis of gene expression in P-TEFb knockdown HeLa cells. 51 down-regulated genes are displayed by class, based on their putative functions. Each row represents one gene. Column 1 indicates hCycT1 ds siRNA treatment and column 2 indicates CDK9 ds siRNA treatment. The brightness of each color reflects the magnitude of the gene expression level (Signal Log Ratio).

FIG. 13 is a chart showing the results of genome-wide analysis of gene expression in P-TEFb knockdown HeLa cells. 39 up-regulated genes are displayed by class, based on their putative functions. Each row represents one gene. Column 1 indicates hCycT1 ds siRNA treatment and column 2 indicates CDK9 ds siRNA treatment. The brightness of each color reflects the magnitude of the gene expression level (Signal Log Ratio).

FIG. 14 is a Western blot demonstrating the kinetics of hCycT1 and CDK9 silencing by siRNA over a 90-hour time course in cells transfected with RFP siRNA (top panel, lanes 1-9), hCycT 1 ds siRNA (lower left panel, lanes 10-18), or CDK9 siRNA (lower right panel, lanes 19-27).

FIG. 15 is the sequence of human Cyclin T1 [SEQ ID NO: 1]. The underlined nucleotides are putative coding regions, and italicized nucleotides are non-coding. Adenine dinucleotides which may be chosen as the start of a potential siRNA target region are in bold.

FIG. 16 is the sequence of human CDK9 [SEQ ID NO: 2]. The underlined nucleotides are putative coding regions, and italicized nucleotides are non-coding. Adenine dinucleotides which may be chosen as the start of a potential siRNA target region are in bold.

FIGS. 17A-C are a chart listing the names, codes, and structures of a number of small molecule inhibitors of CDK9.

FIG. 18 is a Western blot demonstrating the effect of specific silencing of P-TEFb on BCSG-1 protein levels in T47D cells transfected with a mismatch control siRNA (left panel, lanes 1-8) or siRNA specific for hCycT1 (right panel, lanes 9-16).

FIG. 19 is a photograph of a gel demonstrating the effect of specific silencing of P-TEFb on BCSG-1 mRNA levels in T47D cells transfected with siRNA specific for hCycT1.

FIG. 20 is a series of five photomicrographs of T47D cells. Panel a, control mock transfected cells. Panels b and c, cells transfected with siRNA for hCycT1 or CDK9 containing 2 nucleotide mismatches, respectively. Panel d, cells transfected with hCycT1-specific ds siRNA. Panel e, cells transfected with CDK9-specific ds siRNA.

FIG. 21 is two bar graphs illustrating the kinetics of the effect of transfection with hCycT1 ds siRNA on the growth rate of T47D breast cancer cells (left panel) or HeLa cells (right panel).

FIG. 22 is a 3-dimensional bar graph illustrating the effect of specific silencing of P-TEFb by transfection with ds siRNAs on the colony-forming ability of T47D in soft agar.

FIG. 23 is a schematic diagram of a theoretical model for HIV Tat transactivation involving the human P-TEFb (CyclinT1/CDK9) and DSIF/NELF complexes.

FIG. 24 is a schematic illustration of the sequence of Spt5, showing the region and sequences of the sense strands of the wild type [SEQ ID NO:8] and double mismatch (control) [SEQ ID NO:9] siRNAs.

FIG. 25A-B present data showing specific silencing of hSpt5 expression by RNAi. (A) hSpt5 mRNA is 3261 nucleotides in length. siRNA targeting sequence for hSpt5 was selected from position 407 to 427 relative to the start codon. As a specific control, mutant siRNA containing 2 nucleotide mismatches (underline) between the target mRNA and the antisense of siRNA at the hypothetical cleavage sites of the mRNA was generated. (B) Evaluation of specific hSpt5 siRNA activity by RT-PCR. Total cellular mRNA was prepared from HeLa cells transfected without siRNA or with hSpt5 duplex or control siRNAs and was followed by RT-PCR, as described in Material and Methods. Each RT-PCR reaction included 100 ng total cellular mRNA, gene-specific primer sets for hSpt5 and hCycT1 amplification (0.5 μM for each primer), 200 μM dNTP, 1.2 mM MgSO₄ and 1U of RT/platinum Taq mix. Primer sets for hSpt5 produced 2.6 kb products while hCycT1 produced 1.8 kb products. RT-PCR products were resolved in a 1% agarose gel and viewed by ethidium bromide staining. RT-PCR products are shown from cells that were not transfected with siRNA (lane 1), or cells transfected with single-stranded antisense hSpt5 siRNA (hSpt5 (AS), lane 2), hSpt5 duplex siRNA (hSpt5 (DS), lane 3), or mismatch hSpt5 duplex siRNA (hSpt5-mm (DS), lane 4). (C) Analysis of specific hSpt5 siRNA activity by western blotting. Cell lysates were prepared from HeLa cells mock-transfected without siRNA (lane 1), or transfected with single-stranded antisense hSpt5 siRNA (hSpt5 (AS), lane 2), hSpt5 duplex siRNA (hSpt5 (DS), lane 3), or mismatch hSpt5 duplex siRNA (hSpt5-mm (DS), lane 4). Cell lysates were analyzed by 10% SDS-PAGE. Protein contents were detected by immunoblotting assay with antibodies against hSpt5 (top panel) and hCycT1 (lower panel.

FIG. 26A is a representation of a Western blot of protein taken at the time points indicated from HeLa cells transfected with a single strand of anti-sense RNA targeting human Spt5 (hSpt5) (SEQ ID NO:10 TTGACCCGCTCATAATGTACT). Antibodies specific for hSpt5 (upper row) and human Cyclin T1, a subunit of P-TEFb (lower row), were used as indicated.

FIG. 26B is a representation of a Western blot of protein taken at the time points indicated from HeLa cells transfected with double-stranded RNA (dsRNA) [SEQ ID NO:8] targeting Spt5. Antibodies specific for hSpt5 (upper row) and human Cyclin T1, a subunit of P-TEFb (lower row), were used as indicated.

FIG. 26C is a representation of a Western blot of protein taken at the time points indicated from HeLa cells transfected with a control mismatch double-stranded RNA (dsRNA) targeting the sequence of Spt5 with two mismatches [SEQ ID NO.9]. Antibodies specific for hSpt5 (upper row) and human Cyclin T1, a subunit of P-TEFb (lower row), were used as indicated.

FIG. 27 is a line graph depicting the analysis of cell viability by counting trypan blue-stained cells. HeLa cells were transfected with Lipofectamine with various siRNAs or no siRNA as control. Three siRNA duplexes, including hSpt5 siRNA (yellow), mismatch hSpt5 siRNA (light blue) and siRNA targeting human capping enzyme (HCE, red), were used in these experiments. Controls for viability included cells mock-transfected with no siRNA or cells transfected with single-stranded antisense hSpt5 siRNA. At various times after transfection, cells floating in the medium were collected and counted in the presence of 0.2% trypan blue. Cells that took up dye (stained blue) were not viable.

FIG. 28 is a bar graph depicting the effect of hSpt5 siRNA on HIV-1 Tat transactivation in Magi cells. Quantified effect of siRNA on HIV-1 Tat transactivation was determined by β-galactosidase activity assay. Magi cells were cotransfected with pTat-RFP plasmid and various siRNAs targeting hSpt5 or Tat and harvested at 48 h post-transfection. Activity of β-galactosidase was measured using the β-Galactosidase Enzyme Assay System (Promega). Tat transactivation was determined by the ratio of β-galactosidase activity in pTat-RFP transfected cells to activity measured in cells without pTat-RFP. Inhibitory effect of siRNA was determined by normalizing Tat transactivation activity to the amount of Tat-RFP protein. Tat transactivation was measured for Magi cells transfected with pTat-RFP only (lane 1), single-stranded antisense hSpt5 siRNA (hSpt5-AS, lane 2), hSpt5 duplex siRNA (hSpt5-DS, lane 3), mismatch hSpt5 duplex siRNA (hSpt5-mm-DS, lane 4), and Tat duplex siRNA duplex (Tat-DS, lane 5). Results are representative of three independent experiments.

FIG. 29 is a bar graph depicting the effect of hSpt5 silencing by RNAi on Tat transactivation in Magi cells transfected with pTat-RFP plasmid alone (bar 1) or cotransfected with pTat-RFP plasmid and ds siRNAs targeting Spt5 (bar 3), antisense (AS) RNA strands (bar 2), or mutant (mm) siRNAs (bar 4). Means±SD of two experiments are shown.

FIG. 30 is a Western blot showing the effect of Spt5 RNAi activities in otherwise untransfected Magi cells (lanes 1-4) or Magi cells co-transfected with pTat-RFP plasmid (lanes 5-8), using various siRNAs as indicated. The blot was probed with antibodies against hSpt5 (upper panel) and hCycT1 (lower panel).

FIG. 31 is a western blot showing robust knockdown of hSpt5 using a double transfection method. HeLa-CD4-LTR/β-galactosidase (Magi) cells were mock-transfected, or transfected with single-stranded antisense hSpt5 siRNA, hSpt5 duplex siRNA, mismatched hSpt5 duplex siRNA, or Nef duplex siRNA. 24 h after the first transfection, a second siRNA transfection was performed. 24 h later, cells were infected with HIV_{NL-GFP}, an infectious molecular clone of HIV-1. 48 h post-infection, cells were harvested and cell lysates were evaluated for hSpt5 protein levels using hSpt5 antibodies in immunoblot assays (upper panel). Immunoblotting using hCycT1 antibodies (lower panel) was used as an internal control for protein levels. Lanes 1-6 represent knocked down protein levels of cells 96 h after initial transfection, being transfected only once prior to infection. Lanes 7-12 represent knocked down protein levels of cells 96 h after initial transfection, being transfected with siRNA twice prior to infection. Lanes 1 and 7 show protein levels in mock-treated cells not infected with virus after the first and second transfection, respectively. Protein levels are shown from virus-infected cells that had been mock-transfected (lanes 2 and 8), or singly (left panel) or doubly (right panel) transfected with single-stranded antisense hSpt5 siRNA (Spt5 AS, lanes 3 and 9), hSpt5 duplex siRNA (Spt5 (DS), lanes 4 and 10), mismatched hSpt5 duplex siRNA (Spt5-MM (DS), lanes 5 and 11) or Nef duplex siRNA (Nef (DS), lanes 6 and 12).

FIG. 32 is a bar graph depicting that siRNA targeting hSpt5 modulates HIV-1 replication. HeLa-CD4-LTR/β-galactosidase (Magi) cells were mock-transfected (mock), or transfected with single-stranded antisense hSpt5 siRNA (AS), hSpt5 duplex siRNA (siRNA), mismatched hSpt5 duplex siRNA (MM) or Nef duplex siRNA (T98). 24 h after the first transfection, a second siRNA transfection was performed. 24 h later, cells were infected with HIV_{NL-GFP}, an infectious molecular clone of HIV- 1. Cells infected with virus and not treated with oligofectamine are shown (mock). HIV-1 Tat-mediated transactivation of the 5′ LTR led to β-galactosidase production, which was quantified 48 h post-infection. Cells treated with duplex siRNA targeting Nef (lanes marked T98; note that in this clone, Nef is fused to GFP as previously reported in (Jacque et al., 2002 Nature 418:435438) served as a positive control. Serial double dilutions of the viral inoculum (in cpm of RT activity) are consistent with 32-fold decreases in viral replication.

FIG. 33 is a western blot showing the hSpt5 knockdown effect on Hsp40 and Hsp70 expression. Magi cells were transfected without or with hSpt5 duplex siRNA, and 48 h after transfection, cells were incubated under heat shock conditions at 45° C. for 30 min. Cells were harvested at various time points after heat shock and cell lysates were evaluated for protein levels by immunoblot analysis with hSpt5, Hsp40, Hsp70 and hCycT1 antibodies. Protein levels of cells not transfected with hSpt5 siRNA are shown in lanes 1-6. Protein levels of cell transfected with hSpt5 siRNA are shown in lanes 10-15. Time 0 equals the time at which cells began recovery from heat shock. hCycT1 was used an internal control for specificity of hSpt5 knockdown and upregulation of heat shock genes.

FIG. 34 is the sequence of human Spt5 [SEQ ID NO:7]. The italicized nucleotides are non-coding. Adenine dinucleotides that can be chosen as the start of a potential siRNA target region are in bold, and underlined nucleotides are the nucleotides targeted in the present examples.

FIG. 35 is the sequence of mouse Spt5 [SEQ ID NO:11]. The italicized nucleotides are non-coding. Adenine dinucleotides that can be chosen as the start of a potential siRNA target region are in bold, and underlined nucleotides are the nucleotides targeted in the present examples.

FIG. 36 is the sequence of human Spt4 [SEQ ID NO: 12]. The italicized nucleotides are non-coding. Adenine dinucleotides that can be chosen as the start of a potential siRNA target region are in bold.

### DETAILED DESCRIPTION

The human positive transcription elongation factor P-TEFb is composed of two subunits, CyclinT1 (hCycT1) and CDK9, and is involved in transcription regulation of cellular genes as well as HIV-1 mRNA. Replication of HIV-1 requires Tat protein, which activates elongation of RNA polymerase II at the HIV-1 promoter by interacting with hCycT1. To understand the cellular functions of P-TEFb and to test whether suppressing host proteins like P-TEFb can modulate HIV infectivity without causing cellular toxicity or lethality, RNA interference (RNAi) was used to specifically knockdown P-TEFb expression by degrading hCycT1 or CDK9 mRNA. RNAi-mediated gene silencing of P-TEFb in HeLa cells was not lethal and inhibited Tat transactivation and HIV-1 replication in host cells. CDK9 protein stability was found to depend on hCycT1 protein levels, suggesting that formation of P-TEFb CDK-cyclin complexes was required for CDK9 stability. Strikingly, P-TEFb knockdown cells showed normal P-TEFb kinase activity. These studies suggest the existence of a dynamic equilibrium between active and inactive pools of P-TEFb in the cell and this equilibrium is shifted towards the active kinase form to sustain cell viability when P-TEFb protein levels are reduced. The finding that P-TEFb knockdown was not lethal while still showing normal P-TEFb kinase activity suggested that there was a critical threshold concentration of activated P-TEFb required for cell viability and HIV replication. These results provide new insight into the regulation of P-TEFb function and suggest the possibility that cells may use similar mechanisms to modulate other enzymatic pathways.

Based, at least in part, on the above findings, the present invention relates to methods of modulating (e.g., decreasing) the activity of transcription elongation factors (TEFs) and more specifically to ribonucleic acid interference (RNAi) of TEFs (e.g., positive transcription elongation factors or P-TEFs) or subunits thereof (e.g., the P-TEFb subunits CDK9 and CycT1).

The present invention is further based on the discovery that expression of certain transcription elongation regulatory factors (also called TEFs herein) such as DSIF (e.g., DSIF subunits Spt4 and/or Spt5), and Spt6, can be specifically eliminated without fatal consequences to a cell, such as a mammalian cell. Thus, these transcription elongation regulatory factors are targets for treatment of disorders characterized by unwanted or aberrant TEF activity, e.g., CDK9, CycT1, Spt4, Spt5, and/or Spt6 activity, including HIV/AIDS, or disorders characterized by unwanted or aberrant cellular proliferation or differentiation, such as cancer, wherein it is desirable to reduce or eliminate the TEF activity, e.g., CDK9, CycT1, Spt4, Spt5, and/or Spt6 activity, in a cell.

In one embodiment, RNA interference (RNAi) methods are used to specifically silence one or more TEFs, e.g., P-TEFb, DSIF and/or Spt6. These RNAi methods can be used to reduce HIV infectivity and to regulate genes involved in cell proliferation and differentiation, e.g., genes that have been correlated with diseases and disorders characterized by unwanted or aberrant cellular proliferation or differentiation, such as cancer. Furthermore, the use of RNAi technologies to specifically disrupt TEF expression, e.g., P-TEFb, DSIF and/or Spt6 expression, is non-toxic on a cellular level.

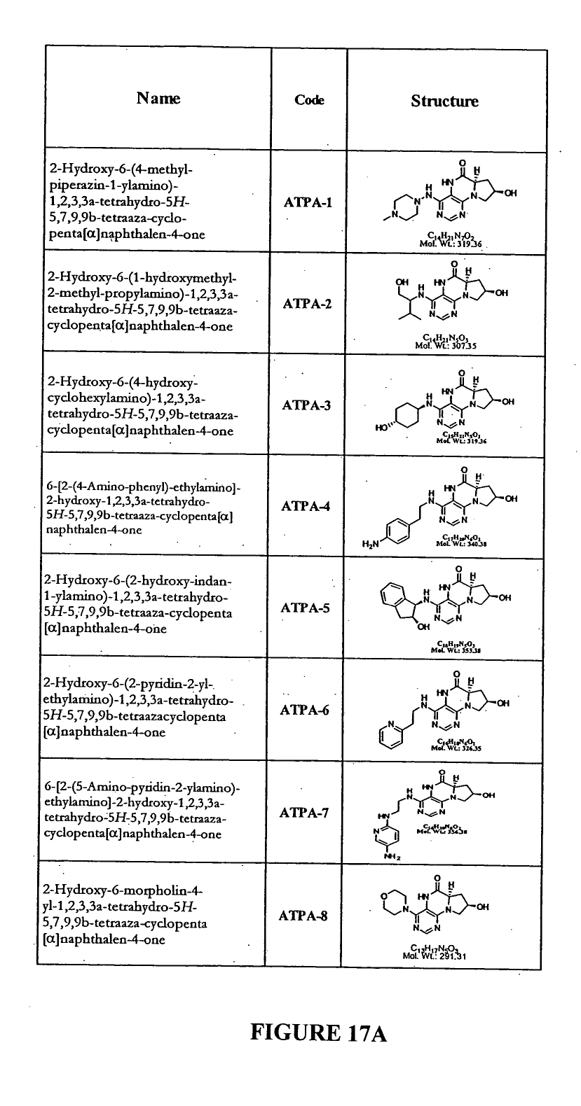

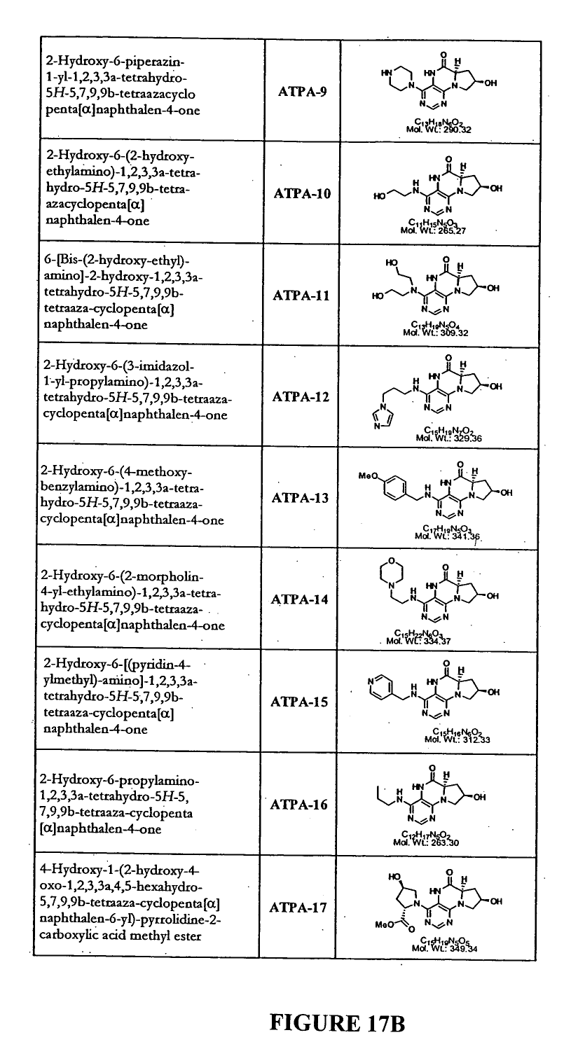

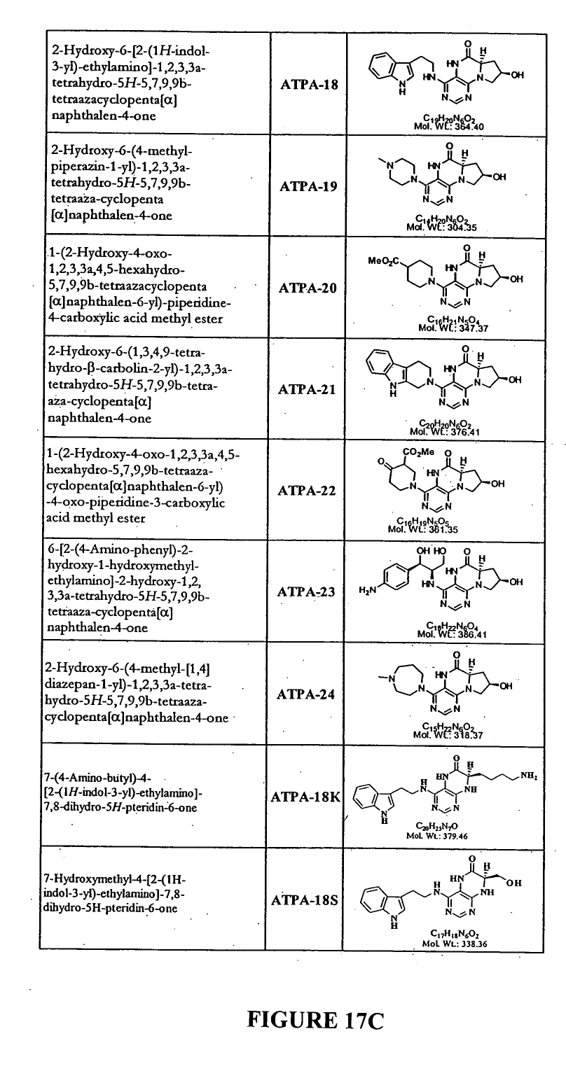

In one aspect, the invention features a method for inhibiting unwanted cellular proliferation in a subject by administering to the subject a therapeutically effective amount of an inhibitor of a TEF, e.g., P-TEFb (CDK9 or CycT1), DSIF (Spt4 or Spt5) or Spt6. In one embodiment, the inhibitor reduces the expression of CDK9, CycT1, Spt4, Spt5, or Spt6. In a further embodiment, the inhibitor can be an antisense nucleotide sequence or strand, a ribozyme, or a siRNA specific for CDK9, CycT1, Spt4, Spt5, or Spt6. In another embodiment, the inhibitor reduces a TEF activity, e.g., P-TEFb (CDK9 or CycT1), DSIF (Spt4 or Spt5), or Spt6 activity by reducing CDK9, CycT1, Spt4, Spt5 or Spt6 activity. In a further embodiment, the inhibitor can be a small molecule, a peptide, or a dominant negative form of CDK9, CycT1, Spt4, Spt5, or Spt6 (e.g., one of the small molecule inhibitors listed in FIG. 17A-C). In one embodiment, the unwanted cellular proliferation is cancer, for instance, carcinomas, sarcomas, metastatic disorders, and hematopoietic neoplastic disorders.

In another aspect, the invention features a method for inhibiting viral replication in a subject by administering to the subject an effective amount of an inhibitor of CDK9, CycT1, Spt4, Spt5, or Spt6. In one embodiment, the inhibitor of CDK9, CycT1, Spt4, Spt5, or Spt6 reduces the expression of CDK9, CycT1, Spt4, Spt5, or Spt6. In a further embodiment, the inhibitor can be an antisense nucleotide sequence or strand, a ribozyme, or an siRNA. In another embodiment, the inhibitor of CDK9, CycT1, Spt4, Spt5, or Spt6 reduces a TEF activity by reducing the activity of CDK9, CycT1, Spt4, Spt5, or Spt6. In a further embodiment, the inhibitor can be a small molecule, a peptide, or a dominant negative form of CDK9, CycT1, Spt4, Spt5, or Spt6 (e.g., one of the small molecule inhibitors listed in FIG. 17A-C). In one embodiment, the viral replication is replication of HIV-1 or HIV-2 or HCV.

In another aspect, the invention features an isolated nucleic acid molecule with a first nucleotide sequence of at least 16 nucleotides substantially identical, e.g., having 3, 2, 1, or 0 mismatches, to a target region of an mRNA sequence of a TEF, e.g., CDK9, CycT1, Spt4, Spt5, or Spt6, and a second nucleotide sequence of at least 16 nucleotides complementary to the first nucleotide sequence. In some embodiments, the mRNA sequence of CycT1 is SEQ ID NO: 1. In some embodiments, the mRNA sequence of CDK9 is SEQ ID NO: 2. In some embodiments, the mRNA sequence of Spt5 is SEQ ID NO: 7 or SEQ ID NO: 11. In some embodiments, the mRNA sequence of Spt4 is SEQ ID NO: 12 or SEQ ID NO: 13. In some embodiments, the mRNA sequence of Spt6 is SEQ ID NO: 14.

In one embodiment, the first nucleotide sequence is fully identical to the mRNA sequence of a TEF, e.g., of CDK9, CycT1, Spt4, Spt5, or Spt6. In another embodiment, the isolated nucleic acid molecule also has a loop portion comprising 4-11, e.g., 4, 5, 6, 7, 8, 9, 10, or 11, nucleotides that connects the two nucleotide sequences. In certain embodiments, the first and second nucleotide sequences comprise 16, 17, 18, 19, 20, 21, 22, 23, 24, 25, 26, 27, 28, 29, or 30 or more nucleotides.

In one embodiment, the target region of the mRNA sequence is located from 100 to 300 nucleotides downstream (3′) of the start of translation of the TEF mRNA. In another embodiment, the target region of the mRNA sequence is located in a 5′ untranslated region (UTR) or a 3′ UTR of the mRNA of a TEF, e.g., CDK9, CycT1, Spt4, Spt5, or Spt6. In a further embodiment, the first or second nucleotide sequence is substantially similar to SEQ ID NO:8. In still further embodiments, the first or second nucleotide sequence is selected from the group consisting of SEQ ID NO: 3 and SEQ ID NO: 5.

In another aspect, the invention features an isolated nucleic acid molecule that encodes a nucleic acid molecule with a first nucleotide sequence of at least 16 nucleotides substantially identical, e.g., having 3, 2, 1, or 0 mismatches, to a target region of an mRNA sequence of a TEF, e.g., CDK9, CycT1, Spt4, Spt5, or Spt6, and a second nucleotide sequence of at least 16 nucleotides complementary to the first nucleotide sequence. In a further embodiment, the first or second nucleotide sequence is SEQ ID NO:9. In yet another embodiment, the first or second nucleotide sequence is SEQ ID NO: 4 or SEQ ID NO:6.

In another aspect, the invention features an expression construct comprising an isolated nucleic acid molecule that encodes a nucleic acid molecule with a first nucleotide sequence of at least 16 nucleotides substantially identical, e.g., having 3, 2, 1, or 0 mismatches, to a target region of an mRNA sequence of CDK9, CycT1, Spt4, Spt5, or Spt6, and a second nucleotide sequence of at least 16 nucleotides complementary to the first nucleotide sequence. In one embodiment, the expression construct is a viral vector, retroviral vector, expression cassette, or plasmid. In another embodiment, the expression construct has an RNA Polymerase III promoter sequence or RNA Polymerase II promoter sequence. In a further embodiment, the RNA Polymerase III promoter is the U6 snRNA promoter or H1 promoter.

In another aspect, the invention provides host cells comprising the nucleic acid molecules of the invention, e.g., the expression constructs of the invention. In one embodiment, the cell is a mammalian cell, e.g., a non-human or human cell.

In another aspect, the invention features therapeutic compositions comprising the nucleic acid molecules of the invention, and a pharmaceutically acceptable carrier.

In another aspect, the invention features methods of treating a subject having a disorder characterized by unwanted cellular proliferation, e.g., cancer, e.g., carcinomas, sarcomas, metastatic disorders and hematopoietic neoplastic disorders (e.g., leukemias), or proliferative skin disorders, e.g., psoriasis, by administering to the subject an amount of a nucleic acid composition, e.g., a therapeutic composition, of the invention, effective to inhibit TEF activity. As used herein, inhibiting P-TEF activity refers to a reduction in the activity of TEF, e.g., by 10%, 20%, 30%, 40%, 50%, 60%, 70%, 80%, 90%, or 100%.

In another aspect, the invention provides a method of treating a subject infected with HIV by administering to the subject an amount of the nucleic acid compositions, e.g., the therapeutic compositions, of the invention, effective to inhibit TEF expression or activity.

In another aspect, the invention features a method of treating a subject having a disorder characterized by aberrant or unwanted expression of a gene whose expression is regulated by a TEF, e.g., CDK9, CycT1, Spt4, Spt5 and/or Spt6, by administering to the subject an amount of the nucleic acid compositions, e.g., the therapeutic compositions, of the invention, effective to inhibit TEF expression or activity. In one embodiment, the gene is selected from the list of genes in Table 1.

In another aspect, the invention features a method of treating a subject having a disorder characterized by aberrant or unwanted expression or activity of a TEF, e.g., CDK9, CycT1, Spt4, Spt5 and/or Spt6 by administering to the subject an amount of the nucleic acid compositions, e.g., the therapeutic compositions, of the invention, effective to inhibit TEF expression or activity. In one embodiment, the disorder is HIV/AIDS. In another embodiment, the disorder is cancer, e.g., carcinomas, sarcomas, metastatic disorders and hematopoietic neoplastic disorders, e.g., leukemia.

So that the invention may be more readily understood, certain terms are first defined.

The term “nucleoside” refers to a molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “RNA” or “RNA molecule” or “ribonucleic acid molecule” refers generally to a polymer of ribonucleotides. The term “DNA” or “DNA molecule” or deoxyribonucleic acid molecule” refers generally to a polymer of deoxyribonucleotides. DNA and RNA molecules can be synthesized naturally (e.g., by DNA replication or transcription of DNA, respectively). RNA molecules can be post-transcriptionally modified. DNA and RNA molecules can also be chemically synthesized. DNA and RNA molecules can be single-stranded (i.e., ssRNA and ssDNA, respectively) or multi-stranded (e.g., double stranded, i.e., dsRNA and dsDNA, respectively). Based on the nature of the invention, however, the term “RNA” or “RNA molecule” or “ribonucleic acid molecule” can also refer to a polymer comprising primarily (i e., greater than 80% or, preferably greater than 90%) ribonucleotides but optionally including at least one non-ribonucleotides molecule, for example, at least one deoxribonucleotide and/or at least one nucleotide analog.

The term “nucleotide analog”, also referred to herein as an “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function.

The term “RNA analog” refers to an polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. As discussed above, the oligonucleotides may be linked with linkages which result in a lower rate of hydrolysis of the RNA analog as compared to an RNA molecule with phosphodiester linkages. Exemplary RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA interference.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of target genes.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference.

A siRNA having a “sequence sufficiently complementary to a target mRNA sequence to direct target-specific RNA interference (RNAi)” means that the siRNA has a sequence sufficient to trigger the destruction of the target mRNA by the RNAi machinery or process. “mRNA” or “messenger RNA” is single-stranded RNA that specifies the amino acid sequence of one or more polypeptide chains. This information is translated during protein synthesis when ribosomes bind to the mRNA.

The term “cleavage site” refers to the residues, e.g. nucleotides, at which RISC* cleaves the target RNA, e.g., near the center of the complementary portion of the target RNA, e.g., about 8-12 nucleotides from the 5′ end of the complementary portion of the target RNA.

The term “mismatch” refers to a basepair consisting of noncomplementary bases, e.g. not normal complementary G:C, A:T or A:U base pairs.

As used herein, the term “isolated” molecule (e.g., isolated nucleic acid molecule) refers to molecules which are substantially free of other cellular material, or culture medium when produced by recombinant techniques, or substantially free of chemical precursors or other chemicals when chemically synthesized.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

A gene “involved” in a disorder includes a gene, the normal or aberrant expression or function of which effects or causes a disease or disorder or at least one symptom of said disease or disorder

Various methodologies of the instant invention include step that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing a siRNA of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Treating HIV Infection

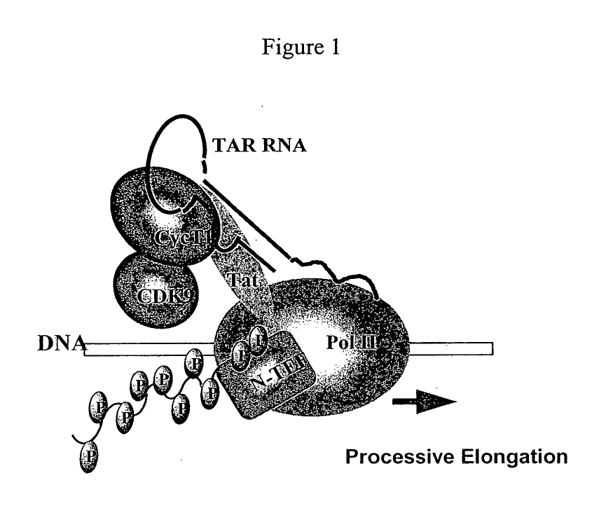

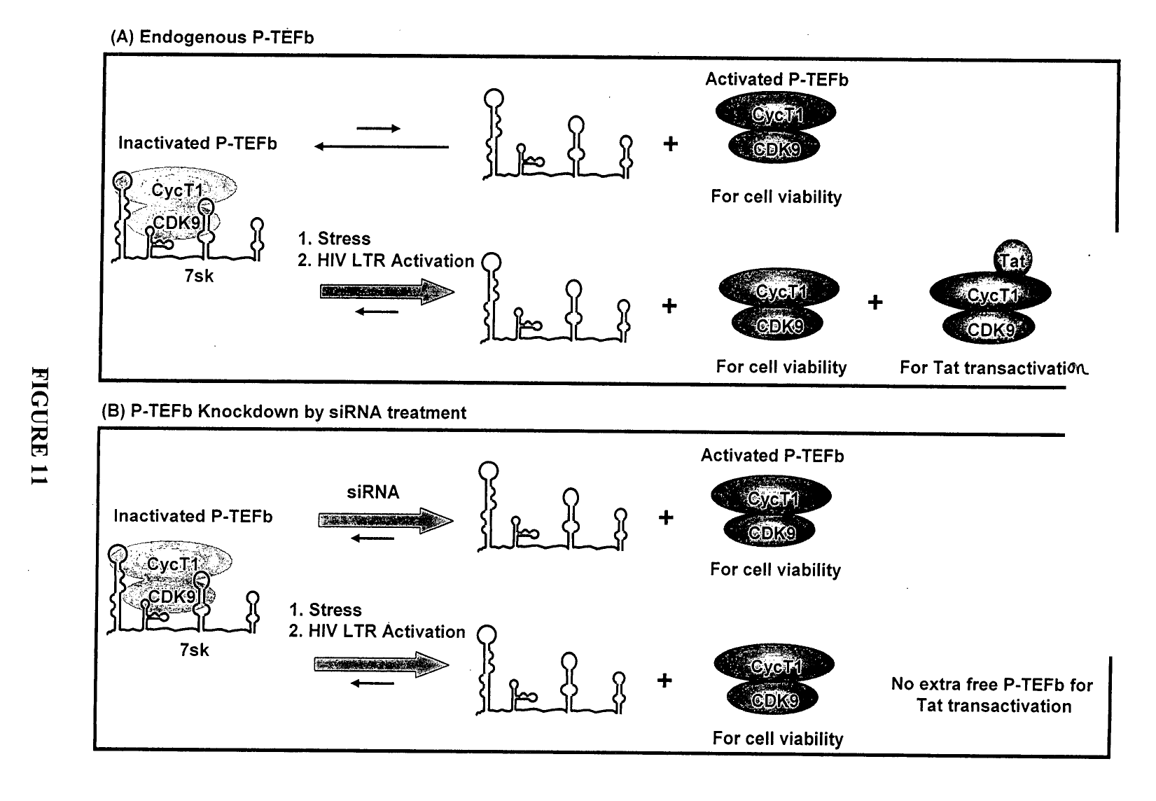

In one aspect, the present invention is based on the discovery that specific reduction of TEF activity, e.g., CDK9, CycT1, Spt4, Spt5 or Spt6 activity, in human cells is non-lethal and can be used to control, e.g., inhibit, Tat transactivation and HIV replication in host cells. While not wishing to be bound by theory, one model for understanding HIV-1 gene regulation is depicted in FIG. 1A and FIG. 11. Briefly, RNA pol II containing nonphosphorylated C-terminal domain (CTD) of the largest subunit (IIA) assembles on the HIV LTR promoter to form a preinitiation complex. TFIIH binds to nonphosphorylated RNA pol II and plays a critical role in transcription initiation and promoter clearance. TFIIH phosphorylates the CTD of the largest subunit of RNA pol II and assists in promoter clearance. The TFIIH complex dissociates from TECs 30 to 50 nucleotides after initiation and is not part of the elongation complexes. P-TEFb, composed of CDK9 and cyclin T1, is a component of PICs, however, it may not be an active kinase at this stage. After promoter clearance, DSIF and NELF associate with the transcription complex during the early elongation stage. Under standard physiological conditions and in the case of non-HIV-1 LTR promoters, Spt5 is phosphorylated by CDK9 once DSIF/NELF associate with the early elongation complex, and this phosphorylation of Spt5 may sufficiently support regular transcription elongation. In the presence of DRB, the kinase activity of CDK9 is inhibited and Spt5 cannot be phosphorylated by P-TEFb. The unphosphorylated form of Spt5 acts as a negative regulator and causes inhibition of RNA pol II elongation. In contrast to cellular promoters, transcription from the HIV-1 LTR promoter is not efficient and CDK9 is activated by Tat protein. In the absence of Tat, elongation complexes which originated at the HIV-1 promoter meet DSIF and NELF, CDK9 is unable to efficiently phosphorylate Spt5 and, as a result, elongation is not processive. After the transcription of a functional TAR RNA structure, Tat binds to TAR and repositions P-TEFb in the vicinity of the CTD of RNA pol II and Spt5. Hyperphosphorylation of the CTD is carried out by P-TEFb after the formation of Tat-TAR-P-TEFb complexes. In addition to CTD phosphorylation, Tat also enhances the phosphorylation of Spt5 mediated by P-TEFb, and the phosphorylated form of Spt5 turns DSIF into a positive regulator of transcription elongation (Ping and Rana, J Biol. Chem., 276:12951-12958 (2001)). Specific reduction in P-TEFb or DSIF activity can be achieved in a number of different ways, including RNAi, antisense, ribozymes, or small molecules targeted to one or both subunits of P-TEFb (e.g., CDK9 or CycT1) or DSIF (e.g., Spt4 or Spt5). Specific reduction in Spt6 activity can be achieved in a number of different ways, including RNAi, antisense, ribozymes, or small molecules targeted to Spt6.

### Treating Cancer

In another aspect, the present invention is based in part on the discovery that specific reduction of transcription elongation factor activity in human cells is non-lethal and can be used to regulate the expression of genes correlated with diseases or disorders characterized by unwanted or aberrant cellular proliferation or differentiation, to decrease the growth of cancerous cells, and reduce the metastatic activity of cancerous cells. Examples of proliferative and/or differentiative disorders include cancer, e.g., carcinomas, sarcomas, metastatic disorders or hematopoietic neoplastic disorders, e.g., leukemias, as well as proliferative skin disorders, e.g., psoriasis or hyperkeratosis. Other myeloproliferative disorders include polycythemia vera, myelofibrosis, chronic myelogenous (myelocytic) leukemia, and primary thrombocythaemia, as well as acute leukemia, especially erythroleukemia, and paroxysmal nocturnal haemoglobinuria. Metastatic tumors can arise from a multitude of primary tumor types, including but not limited to those of prostate, colon, lung, breast and liver origin. Specific reduction in transcription elongation factors such as P-TEFb (CDK9/CycT1), DSIF (Spt4/Spt5) or Spt6, can be achieved in a number of different ways, including the introduction into a cell of RNAi, antisense, ribozyme, dominant negative mutation or sequences containing such mutation, or small molecules targeted to the factor, e.g., one or both subunits of P-TEFb (CDK9/CycT1), one or both subunits of DSIF (e.g., Spt5 or Spt4) or Spt6.

### RNA Interference

RNAi is a remarkably efficient process whereby double-stranded RNA (dsRNA, also referred to herein as siRNAs or ds siRNAs, for double-stranded small interfering RNAs,) induces the sequence-specific degradation of targeted mRNA in animals and plant cells (Hutvagner and Zamore, Curr. Opin. Genet. Dev.: 12, 225-232 (2002); Sharp, Genes Dev., 15:485-490 (2001)). In mammalian cells, RNAi can be triggered by 21-nucleotide (nt) duplexes of small interfering RNA (siRNA) (Chiu et al., Mol. Cell. 10:549-561 (2002); Elbashir et al., Nature 411:494-498 (2001)), or by micro-RNAs (miRNA), functional small-hairpin RNA (shRNA), or other dsRNAs which can be expressed in vivo using DNA templates with RNA polymerase III promoters (Zeng et al., Mol. Cell 9:1327-1333 (2002); Paddison et al., Genes Dev. 16:948-958 (2002); Lee et al., Nature Biotechnol. 20:500-505 (2002); Paul et al., Nature Biotechnol. 20:505-508 (2002); Tuschl, T., Nature Biotechnol. 20:440-448 (2002); Yu et al., Proc. Natl. Acad. Sci. USA 99(9):6047-6052 (2002); McManus et al., RNA 8:842-850 (2002); Sui et al., Proc. Natl. Acad. Sci. USA 99(6):5515-5520 (2002).)

Accordingly, the invention includes such molecules that are targeted to a CDK9, CycT1, Spt4, Spt5, or Spt6 RNA.

### siRNA Molecules

The nucleic acid molecules or constructs of the invention include dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA of CDK9 [SEQ ID NO: 1], CycT1 [SEQ ID NO: 2], Spt4 [SEQ ID NO: 12 or SEQ ID NO: 13], Spt5 [SEQ ID NO: 7 or SEQ ID NO: 11], or Spt6 [SEQ ID NO: 14], and the other strand is identical or substantially identical to the first strand. The dsRNA molecules of the invention can be chemically synthesized, or can be transcribed in vitro from a DNA template, or in vivo from, e.g., shRNA. The dsRNA molecules can be designed using any method known in the art, for instance, by using the following protocol:

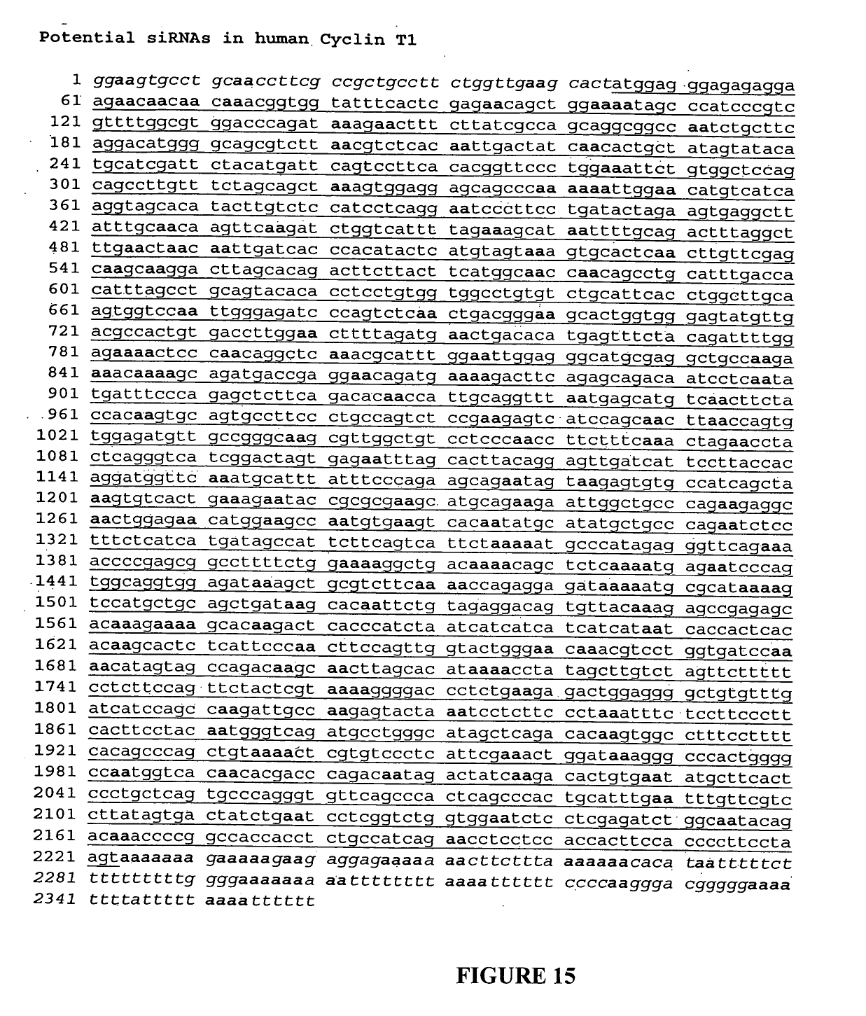

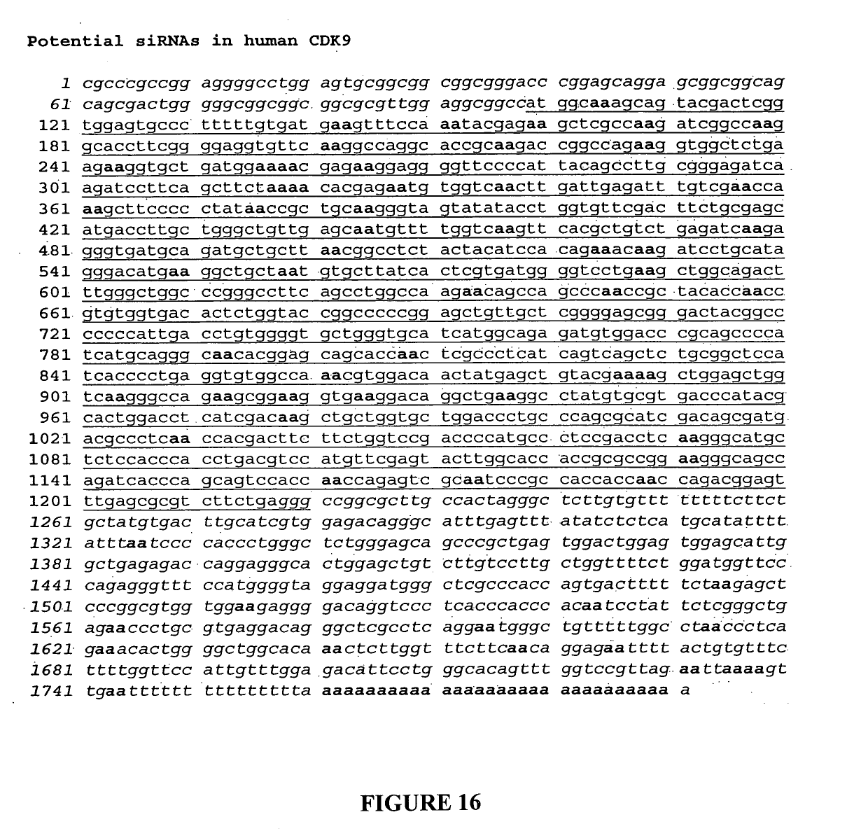

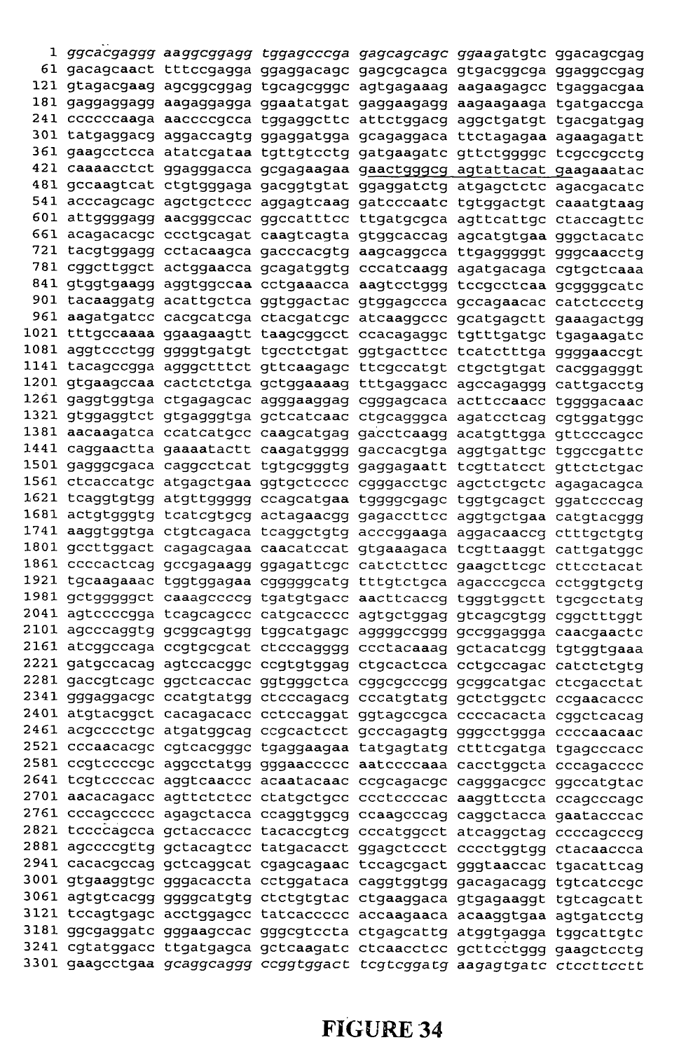

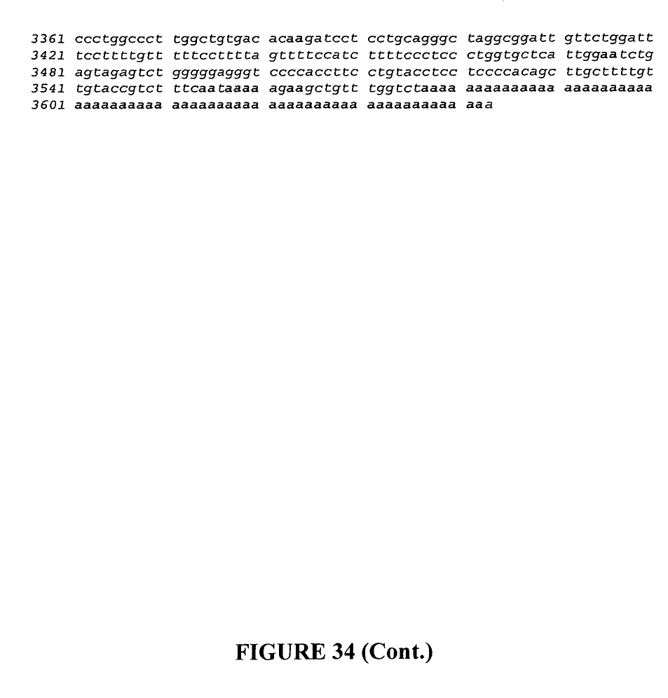

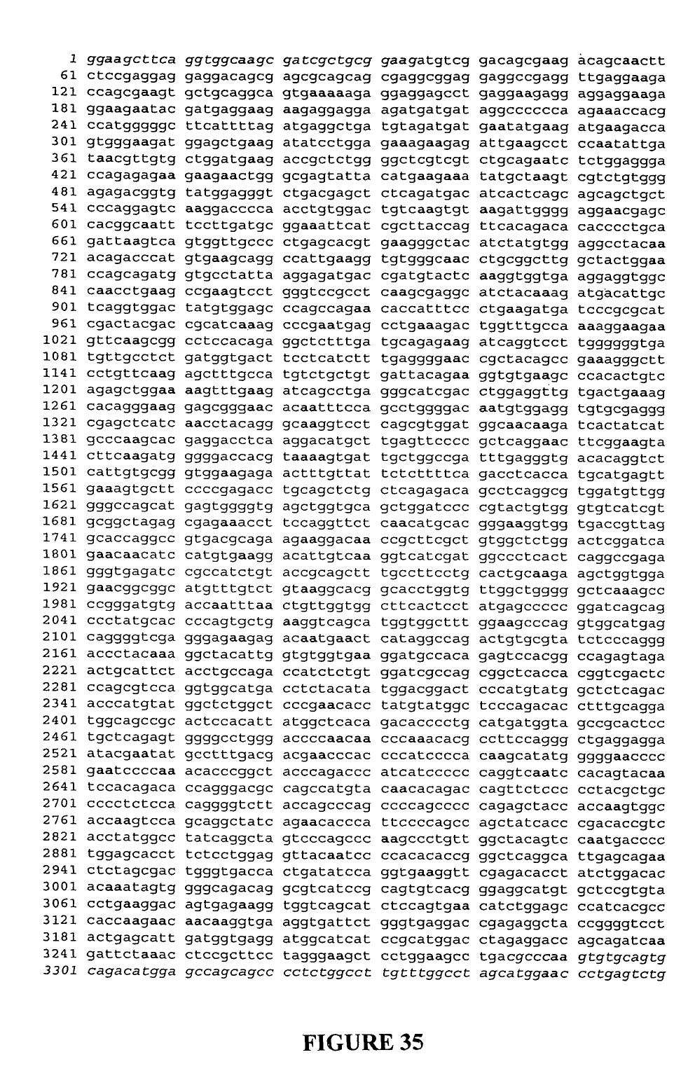

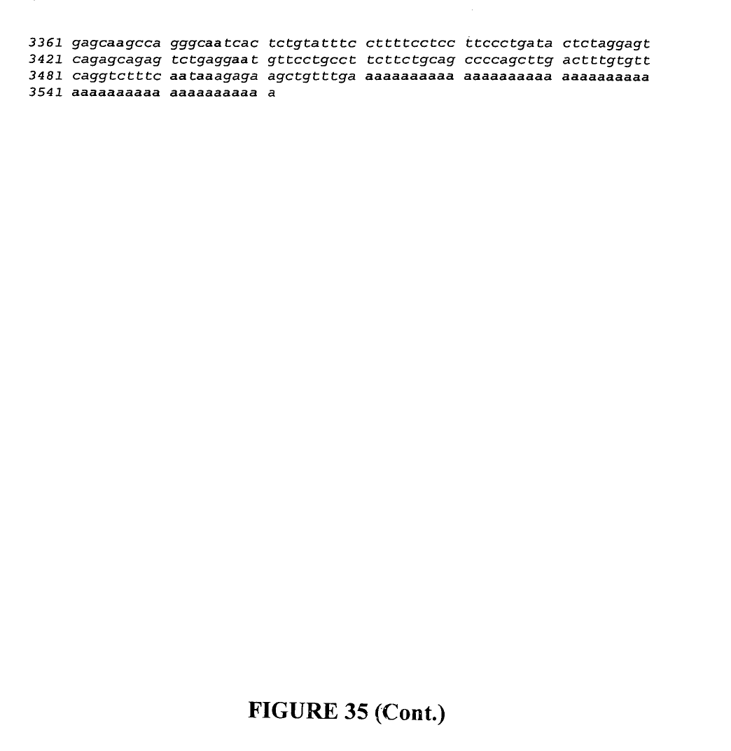

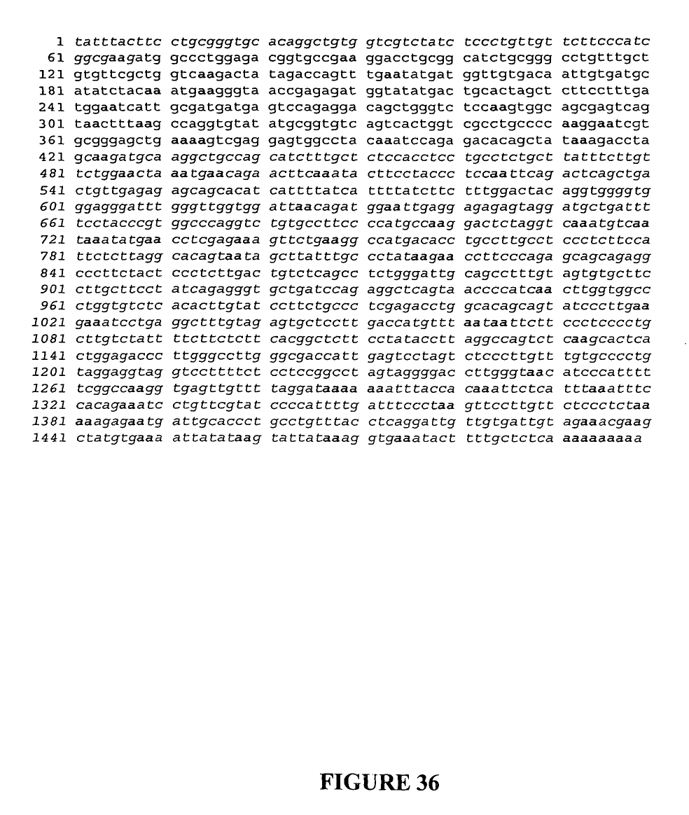

1. Beginning with the AUG start codon, look for AA dinucleotide sequences; each AA and the 3′ adjacent 16 or more nucleotides are potential siRNA targets (see FIGS. 15, 16, 34, 35, 36). siRNAs taken from the 5′ untranslated regions (UTRs) and regions near the start codon (within about 75 bases or so) may be less useful as they may be richer in regulatory protein binding sites, and UTR-binding proteins and/or translation initiation complexes may interfere with binding of the siRNP or RISC endonuclease complex. Thus, in one embodiment, the nucleic acid molecules are selected from a region of the cDNA sequence beginning 50 to 100 nt downstream of the start codon. Further, siRNAs with lower G/C content (35-55%) may be more active than those with G/C content higher than 55%. Thus in one embodiment, the invention includes nucleic acid molecules having 35-55% G/C content. In addition, the strands of the siRNA can be paired in such a way as to have a 3′ overhang of 1 to 4, e.g., 2, nucleotides. Thus in another embodiment, the nucleic acid molecules can have a 3′ overhang of 2 nucleotides, such as TF. The overhanging nucleotides can be either RNA or DNA.

2. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at the National Center for Biotechnology Information web site of the National Institutes of Health.

3. Select one or more sequences that meet your criteria for evaluation. Further general information about the design and use of siRNA can be found in “The siRNA User Guide,” available at the web site of the laboratory of Dr. Thomas Tuschl at Rockefeller University.

Negative control siRNAs should have the same nucleotide composition as the selected siRNA, but without significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The nucleic acid compositions of the invention include both unmodified TEF siRNAs and modified TEF siRNAs as known in the art, such as crosslinked siRNA derivatives as described in U.S. Provisional Patent Application 60/413,529, which is incorporated herein by reference in its entirety. Crosslinking can be employed to alter the pharmacokinetics of the composition, for example, to increase half-life in the body. Thus, the invention includes siRNA derivatives that include siRNA having two complementary strands of nucleic acid, such that the two strands are crosslinked. For example, a 3′ OH terminus of one of the strands can be modified, or the two strands can be crosslinked and modified at the 3′OH terminus. The siRNA derivative can contain a single crosslink (e.g., a psoralen crosslink). In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying SiRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acid compositions of the invention can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al., Drug Deliv. Rev.:47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

The nucleic acid molecules of the present invention can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using ³H,³²P, or other appropriate isotope.

The dsRNA molecules of the present invention can comprise the following sequences as one of their strands, and the corresponding sequences of allelic variants thereof:

| | | |
|---|---|---|
| hCycT1 ds | SEQ ID NO: 3 | 5′-UCCCUUCCUGAUACUAGAAdTdT- |
| | | 3′ |
| HcycT1 mm | SEQ ID NO: 4 | 5′-UCCCUUCCGUAUACUAGAAdTdT- |
| (neg. ctrl) | | 3′ |
| CDK9 ds | SEQ ID NO: 5 | 5′-CCAAAGCUUCCCCCUAUAAdTdT- |
| | | 3′ |
| CDK9 mm | SEQ ID NO: 6 | 5′-CCAAAGCUCUCCCCUAUAAdTdT- |
| (neg. ctrl) | | 3′ |
| Spt5 ds | SEQ ID NO: 8 | 5′-AACTGGGCGAGTATTACATGAdTd |
| | | T-3 |
| Spt5 mm | SEQ ID NO: 9 | 5′-AACTGGGCGGATATTACATGAdTd |
| (neg. ctrl) | | T-3′ |

SEQ ID NOs: 3, 4, 5, 6, 8 and 9 (e.g., sense sequences) correspond to targeted portions of their target mRNAs, as described herein. For convenience, reverse complementary sequences (e.g., antisense sequences) are set forth as SEQ ID NOs: 21, 22, 23, 24, 25 and 26, respectively. dsRNA molecules of the present invention preferably comprise one of SEQ ID NOs: 3, 4, 5, 6, 8 and 9 paired with one of SEQ ID NOs: 21, 22, 23, 24, 25 and 26, respectively.

Moreover, because RNAi is believed to progress via at least one single stranded RNA intermediate, the skilled artisan will appreciate that ss-siRNAs (e.g., the antisense strand of a ds-siRNA) can also be designed as described herein and utilized according to the claimed methodologies.

### siRNA Delivery for Longer-Term Expression

Synthetic siRNAs can be delivered into cells by methods known in the art, including cationic liposome transfection and electroporation. However, these exogenous siRNA generally show short term persistence of the silencing effect (4˜5 days in cultured cells), which may be beneficial in certain embodiments. To obtain longer term suppression of TEFs and to facilitate delivery under certain circumstances, one or more siRNA duplexes, e.g., CDK(, CycT1, Spt4, Spt5, or Spt6 ds siRNA, can be expressed within cells from recombinant DNA constructs. Such methods for expressing siRNA duplexes within cells from recombinant DNA constructs to allow longer-term target gene suppression in cells are known in the art, including mammalian Pol III promoter systems (e.g., H1 or U6/snRNA promoter systems (Tuschl (2002), supra) capable of expressing functional double-stranded siRNAs; (Bagella et al., J. Cell. Physiol. 177:206-213 (1998); Lee et al. (2002), supra; Miyagishi et al. (2002), supra; Paul et al. (2002), supra; Yu et al. (2002), supra; Sui et al. (2002), supra). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence under the control of T7 promoter also make fuinctional siRNAs when cotransfected into the cells with a vector expressing T7 RNA polymerase (Jacque (2002), supra). A single construct may contain multiple sequenc3es coding for siRNAs, such as multiple regions of CDK9, CycT1, Spt4, Spt5, and/or Spt6, targeting the same gene or multiple genes, and can be driven, for example, by separate PolIII promoter sites.

Animal cells express a range of noncoding RNAs of approximately 22 nucleotides termed micro RNA (miRNAs) which can regulate gene expression at the post transcriptional or translational level during animal development. One common feature of miRNAs is that they are all excised from an approximately 70 nucleotide precursor RNA stem-loop, probably by Dicer, an RNase III-type enzyme, or a homolog thereof. By substituting the stem sequences of the miRNA precursor with miRNA sequence complementary to the target mRNA, a vector construct that expresses the novel miRNA can be used to produce siRNAs to initiate RNAi against specific mRNA targets in mammalian cells (Zeng (2002), supra). When expressed by DNA vectors containing polymerase III promoters, micro-RNA designed hairpins can silence gene expression (McManus (2002), supra). Viral-mediated delivery mechanisms can also be used to induce specific silencing of targeted genes through expression of siRNA, for example, by generating recombinant adenoviruses harboring siRNA under RNA Pol II promoter transcription control (Xia et al. (2002), supra). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. Id. In an animal model, whole-embryo electroporation can efficiently deliver synthetic siRNA into post-implantation mouse embryos (Calegari et al., Proc. Natl. Acad. Sci. USA 99(22):14236-40 (2002)). In adult mice, efficient delivery of siRNA can be accomplished by “high-pressure” delivery technique, a rapid injection (within 5 seconds) of a large volume of siRNA containing solution into animal via the tail vein (Liu (1999), supra; McCaffrey (2002), supra; Lewis, Nature Genetics 32:107-108 (2002)). Nanoparticles and liposomes can also be used to deliver siRNA into animals.

### Uses of Engineered RNA Precursors to Induce RNAi

Engineered RNA precursors, introduced into cells or whole organisms as described herein, will lead to the production of a desired siRNA molecule. Such an siRNA molecule will then associate with endogenous protein components of the RNAi pathway to bind to and target a specific mRNA sequence for cleavage and destruction. In this fashion, the mRNA to be targeted by the siRNA generated from the engineered RNA precursor will be depleted from the cell or organism, leading to a decrease in the concentration of the protein encoded by that mRNA in the cell or organism. The RNA precursors are typically nucleic acid molecules that individually encode either one strand of a dsRNA or encode the entire nucleotide sequence of an RNA hairpin loop structure.

### Antisense

An “antisense” nucleic acid can include a nucleotide sequence that is complementary to a “sense” nucleic acid encoding a protein, e.g., complementary to the coding strand of a double-stranded cDNA molecule or complementary to a TEF mRNA sequence. The antisense nucleic acid can be complementary to an entire coding strand of a target sequence, or to only a portion thereof (for example, the coding region of human CDK9, corresponding to SEQ ID NO:2, the coding region of human CycT1, corresponding to SEQ ID NO:1, the coding region of human Spt5 corresponding to SEQ ID NO:7 or mouse Spt5 corresponding to SEQ ID NO:1 1). In another embodiment, the antisense nucleic acid molecule is antisense to a “noncoding region” of the coding strand of a nucleotide sequence encoding CDK9, CycT1, Spt4, Spt5, or Spt6 (e.g., the 5′ and 3′ untranslated regions). For example, the antisense oligonucleotide can be complementary to the region surrounding the translation start site of CDK9 or CycT1 mRNA, e.g., between the −10 and +10 regions of the translation start site of the target gene nucleotide sequence of interest, e.g., CDK9 [ggaggcggccatggcaaagc: SEQ ID NO: 15] or CycT1 [tgaagcactatggagggaga: SEQ ID NO:16]. An antisense oligonucleotide can be, for example, about 7, 10, 15, 20, 25, 30, 35, 40, 45, 50, 55, 60, 65, 70, 75, 80, or more nucleotides in length.

An antisense nucleic acid can be designed such that it is complementary to the entire coding region of a target TEF mRNA, e.g., CDK9, CycT1, Spt4, Spt5, or Spt6 mRNA, but can also be an oligonucleotide that is antisense to only a portion of the coding or noncoding region of the target mRNA. For example, the antisense oligonucleotide can be complementary to the region surrounding the translation start site of the target mRNA, e.g., between the -10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject (e.g., by direct injection at a tissue site), or generated in situ such that they hybridize with or bind to cellular mRNA and/or genomic DNA encoding a CDK9, CycT1, Spt4, Spt5, or Spt6 protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al., Nucleic Acids. Res. 15:6625-6641 (1987)). The antisense nucleic acid molecule can also comprise a 2′-o-methylribonucleotide (Inoue et al. Nucleic Acids Res. 15:6131-6148 (1987)) or a chimeric RNA-DNA analogue (Inoue et al. FEBS Lett., 215:327-330 (1987)).

CDK9, CycT1, Spt4, Spt5, or Spt6 gene expression can be inhibited by targeting nucleotide sequences complementary to the regulatory region of CDK9, CycT1, Spt4, Spt5, or Spt6 (e.g., the CDK9, CycT1, Spt4, Spt5, or Spt6 promoters and/or enhancers) to form triple helical structures that prevent transcription of the CDK9, CycT1, Spt4, Spt5, or Spt6 gene in target cells. See generally, Helene, C. Anticancer Drug Des. 6:569-84 (1991); Helene, C. Ann. N.Y. Acad. Sci. 660:27-36 (1992); and Maher, Bioassays 14:807-15 (1992). The potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

### Ribozymes

Ribozymes are a type of RNA that can be engineered to enzymatically cleave and inactivate other RNA targets in a specific, sequence-dependent fashion. By cleaving the target RNA, ribozymes inhibit translation, thus preventing the expression of the target gene. Ribozymes can be chemically synthesized in the laboratory and structurally modified to increase their stability and catalytic activity using methods known in the art. Alternatively, ribozyme genes can be introduced into cells through gene-delivery mechanisms known in the art. A ribozyme having specificity for a CDK9-, CycT1-, Spt4-, Spt5-, or Spt6-encoding nucleic acid can include one or more sequences complementary to the nucleotide sequence of an CDK9, CycT1, Spt4, Spt5, or Spt6 cDNA disclosed herein (i.e., SEQ ID NO:2, SEQ ID NO:1, SEQ ID NO:7, SEQ ID NO:11, SEQ ID NO:12, SEQ ID NO:13, or SEQ ID NO:14), and a sequence having known catalytic sequence responsible for mRNA cleavage (see U.S. Pat. No. 5,093,246 or Haselhoff and Gerlach Nature 334:585-591 (1988)). For example, a derivative of a Tetrahymena L-19 IVS RNA can be constructed in which the nucleotide sequence of the active site is complementary to the nucleotide sequence to be cleaved in a CDK9-, CycT1-, Spt4-, Spt5-, or Spt6-encoding mRNA. See, e.g., Cech et al. U.S. Pat. No. 4,987,071; and Cech et al. U.S. Pat. No. 5,116,742. Alternatively, CDK9, CycT1, Spt4, Spt5, or Spt6 mRNA can be used to select a catalytic RNA having a specific ribonuclease activity from a pool of RNA molecules. See, e.g., Bartel, D. and Szostak, J. W. Science 261:1411-1418 (1993).

### TEF Nucleic Acid Targets

The nucleic acid targets of the antisense, RNAi, and ribozymes as described herein may be any TEF, including but not limited to CDK9, CycT1, Spt4, Spt5, and/or Spt6. In some embodiments, the nucleic acid target is an mRNA for CDK9, CycT1, Spt4, Spt5, or Spt6.

The mRNA sequence of CDK9 can be any ortholog of CDK9, such as sequences substantially identical to the S. cerevisiae, human, C. elegans, D. melanogaster, or mouse CDK9, including but not limited to GenBank Accession Nos. NM_{—}001261 (GI:17017983) (SEQ ID NO:2) (corresponding protein sequence: NP_{—}001252) (human); P50750 (human); NP_{—}570930 (mouse); BA C40824 (mouse); NP_{—}477226 (fruit fly); NP_{—}492906 (C. elegans); or NP_{—}492907 (C. elegans). The mRNA sequence of CycT1 can be any ortholog of CycT1, such as sequences substantially identical to the S. cerevisiae, human, or mouse CycT1, including but not limited to GenBank Accession Nos. AF048730 (GI:2981195) (SEQ ID NO:1) (corresponding protein sequence: AAC39664) (human); NM_{—}001240 (GI:17978465) (corresponding protein sequence: NP_{—}001231) (human); AAN73282 (chimpanzee); NP_{—}033963 (mouse); AAD17205 (mouse); QDQWV9 (mouse); AAM74155 (goat); or AAM74156 (goat).

In some embodiments, the mRNA sequence of CDK9 can be SEQ ID NO:2 or an ortholog thereof. In some embodiments, the mRNA sequence of CycT1 can be SEQ ID NO: 1 or an ortholog thereof.

The mRNA sequence of Spt4 can be any ortholog of Spt4, such as sequences substantially identical to the S. cerevisiae, human, or mouse Spt4, including but not limited to GenBank Accession Nos. NM 003168 (GI:4507310) (SEQ ID NO: 12) (human Spt4); U38817 (GI:1401054) (humanSpt4); U38818 (GI:1401052) (human Spt4); U43923 (GI:1297309)(human Spt4); NM 009296 (GI:6678180) (SEQ ID NO:13) (mouse Spt4); U43154 (GI:1401065) (mouse Spt4) or M83672 (S. cerevisiae Spt4). The mRNA sequence of Spt5 can be any ortholog of Spt5, such as sequences substantially identical to the S. cerevisiae, human, or mouse Spt5, including but not limited to GenBank Accession Nos. BC02403 (GI: 18848307) (SEQ ID NO:7) (human Spt5), NM 003169 (GI:20149523) (human Spt5); AB000516 (GI:2723379) (human Spt5); AF 040253 (GI:4104823) (human Spt5); U56402 (GI:1845266) (human Spt5); NM013676 (GI:22094122) (SEQ ID NO:11) (mouse Spt5); U888539 (mouse Spt5); or M 62882 (S. cerevisiae Spt5). The mRNA sequence of Spt6 can be any ortholog of Spt6, such as sequences substantially identical to the S. cerevisiae or mouse Spt6, including but not limited to NM 009297 (GI:6678182) (SEQ ID NO:14) (mouse Spt6) or M34391 (S. cerevisiae Spt6).

In some embodiments, the mRNA sequence of Spt5 is SEQ ID NO:7 or SEQ ID NO:11 or an ortholog thereof. In some embodiments, the mRNA sequence of Spt4 is SEQ ID NO:12 or SEQ ID NO:13 or an ortholog thereof. In some embodiments, the mRNA sequence of Spt6 is SEQ ID NO: 14 or an ortholog thereof.

The term “ortholog” as used herein refers to a sequence which is substantially identical to a reference sequence. The term “substantially identical” is used herein to refer to a first amino acid or nucleotide sequence that contains a sufficient or minimum number of identical or equivalent (e.g., with a similar side chain) amino acid residues or nucleotides to a second amino acid or nucleotide sequence such that the first and second amino acid or nucleotide sequences have a common structural domain or common functional activity. For example, amino acid or nucleotide sequences that. contain a common structural domain having at least about 60%, or 65% identity, likely 75% identity, more likely 85%, 90%. 91%, 92%, 93%, 94%, 95%, 96%, 97%, 98% or 99% identity are defined herein as substantially identical.

Calculations of homology or sequence identity between sequences (the terms are used interchangeably herein) are performed as follows.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 50%, at least 60%, at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In one embodiment, the percent identity between two amino acid sequences is determined using the Needleman and Wunsch (J. Mol. Biol. 48:444-453 (1970)) algorithm which has been incorporated into the GAP program in the GCG software package (available at the official Accelrys web site), using either a Blossum 62 matrix or.a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (available at the official Accelrys web site), using a NWSgapdna.CMP matrix and a gap weight of 40, 50, 60, 70, or 80 and a length weight of 1, 2, 3, 4, 5, or 6. One set of parameters (and the one that can be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

The percent identity between two amino acid or nucleotide sequences can be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid and protein sequences described herein can be used as a “query sequence” to perform a search against public databases to, for example, identify other orthologs, e.g., family members or related sequences. Such searches can be performed using the NBLAST and XBLAST programs (version 2.0) of Altschul, et al. J. Mol. Biol. 215:403-10 (1990). BLAST nucleotide searches can be performed with the NBLAST program, score=100, wordlength=12 to obtain nucleotide sequences homologous to known TEF nucleic acid molecules of the invention. BLAST protein searches can be performed with the XBLAST program, score=50, wordlength=3 to obtain amino acid sequences homologous to known TEF protein molecules. To obtain gapped alignments for comparison purposes, Gapped BLAST can be utilized as described in Altschul et al., Nucleic Acids Res. 25:3389-3402 (1997). When utilizing BLAST and Gapped BLAST programs, the default parameters of the respective programs (e.g., XBLAST and NBLAST) can be used. See the National Center for Biotechnology Information web site of the National Institutes of Health.

Orthologs can also be identified using any other routine method known in the art, such as screening a cDNA library, e.g., a human cDNA library, using a probe designed to identify sequences which are substantially identical to a reference sequence.

### Small Molecules

Small molecule inhibitors can also be used to specifically reduce TEF activity. In one embodiment, small molecule inhibitors of Spt4, Spt5, or Spt6 can be used to reduce DSIF or Spt6 activity. In another embodiment, small molecule inhibitors of CDK9 or CycT1 can be used to reduce P-TEFb activity. In an exemplary embodiment, the small molecule inhibitors bind CDK9 or CycT1 to inhibit or inactivate P-TEFb. Small molecules include, but are not limited to, peptides, peptidomimetics (e.g., peptoids), amino acids, amino acid analogs, polynucleotides, polynucleotide analogs, nucleotides and/or nucleotide analogs. Preferably, the term “small molecule” refers to organic or inorganic compounds (i.e.,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds. The small molecules can be, for example, any of the small molecule inhibitors listed in FIG. 17A-C.

The small molecules can be obtained using any of numerous approaches in the art. For example, assaying combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., J. Med. Chem., 37:2678-85 (1994)); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, K. S., Anticancer Drug Des. 12:145 (1997)).

Examples of methods for the synthesis of molecular libraries can be found in the art, for example in: DeWitt et al., Proc. Natl. Acad. Sci. U.S.A. 90:6909 (1993); Erb et al., Proc. Natl. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33:2059 (1994); Carell et al., Angew. Chem. Int. Ed. Engl. 33:2061 (1994); and Gallop et al., J. Med. Chem. 37:1233 (1994).

Libraries of compounds can be presented in solution (e.g., Houghten, Biotechniques 13:412-421 (1992)), or on beads (Lam, Nature 354:82-84 (1991)), chips (Fodor, Nature 364:555-556 (1993)), bacteria (Ladner, U.S. Pat. No. 5,223,409), spores (Ladner U.S. Pat. No. '409), plasmids (Cull et al., Proc Natl Acad Sci USA 89:1865-1869 (1992)) or on phage (Scott and Smith, Science 249:386-390 (1990); Devlin, Science 249:404-406 (1990); Cwirla et al., Proc. Natl. Acad. Sci. 87:6378-6382 (1990); Felici, J. Mol. Biol. 222:301-310 (1991); Ladner supra.).

Any cell-based or cell-free assay known in the art can be used to determine the ability of a small molecule to modulate TEF activity, e.g., P-TEFb (e.g., CDK9 or CycT1), DSIF (e.g. Spt4 or Spt5) and/or Spt6 activity. Determining the ability of the small molecule to modulate TEF activity, e.g., P-TEFb (e.g., CDK9 or CycT1), DSIF (e.g. Spt4 or Spt5) and/or Spt6 activity can be accomplished by monitoring, for example, transcription, e.g., transcription of a reporter gene, or by any other assay known in the art.

### Pharmaceutical Compositions and Methods of Administration

The siRNA molecules of the invention, antisense strands, ribozymes, and small molecules, can be incorporated into pharmaceutical compositions. Such compositions typically include the nucleic acid molecule and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. Isotonic agents, for example, sugars, polyalcohols such as manitol, sorbitol, sodium chloride can also be included in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, suitable methods of preparation include vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

The compounds can also be administered by transfection or infection using methods known in the art, including but not limited to the methods described in McCaffrey et al., Nature 418(6893):38-39 (2002) (hydrodynamic transfection); Xia et al., Nature Biotechnol. 20(10):1006-10 (2002) (viral-mediated delivery); or Putnam, Am. J. Health Syst. Pharm. 53(2):151-160 (1996), erratum at Am. J. Health Syst. Pharm. 53(3):325 (1996).

The compounds can also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol. 88(2):205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices can be used. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds generally lies within a range of circulating concentrations that include the ED50 with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography.

As defined herein, a therapeutically effective amount of a nucleic acid molecule (i.e., an effective dosage) depends on the nucleic acid selected. For instance, if a plasmid encoding shRNA is selected, single dose amounts in the range of approximately 1 :g to 10000 mg can be administered; in some embodiments, 10, 30, 100 or 1000 :g can be administered. In some embodiments, 1 g of the compositions can be administered. The compositions can be administered on any appropriate schedule, e.g., from one or more times per day to one or more times per week; including once every other day, for any number of days or weeks, e.g., 1 day, 2 days, 3 days, 4 days, 5 days, 6 days, 1 week, 10 days, 2 weeks, 3 weeks, 4 weeks, 5 weeks, 6 weeks, 7 weeks, 8 weeks, 2 months, 3 months, 6 months, or more, or any variation thereon. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or can include a series of treatments.

The nucleic acid molecules of the invention can be inserted into expression constructs, e.g., viral vectors, retroviral vectors, expression cassettes, or plasmid viral vectors, e.g., using methods known in the art, including but not limited to those described in Xia et al., (2002), supra. Expression constructs can be delivered to a subject by, for example, inhalation, orally, intravenous injection, local administration (see U.S. Pat. No. 5,328,470) or by stereotactic injection (see e.g., Chen et al., Proc. Natl. Acad. Sci. USA 91:3054-3057 (1994)). The pharmaceutical preparation of the delivery vector can include the vector in an acceptable diluent, or can comprise a slow release matrix in which the delivery vehicle is imbedded. Alternatively, where the complete delivery vector can be produced intact from recombinant cells, e.g., retroviral vectors, the pharmaceutical preparation can include one or more cells which produce the gene delivery system.

The nucleic acid molecules of the invention can also include small hairpin RNAs (shRNAs), and expression constructs engineered to express shRNAs. Transcription of shRNAs is initiated at a polymerase III (pol III) promoter, and is thought to be terminated at position 2 of a 4-5-thymine transcription termination site. Upon expression, shRNAs are thought to fold into a stem-loop structure with 3′ UU-overhangs; subsequently, the ends of these shRNAs are processed, converting the shRNAs into siRNA-like molecules of about 21 nucleotides. Brummelkamp et al., Science 296:550-553 (2002); Lee et al, (2002). supra; Miyagishi and Taira, Nature Biotechnol. 20:497-500 (2002); Paddison et al. (2002), supra; Paul (2002), supra; Sui (2002) supra; Yu et al. (2002), supra. More information about shRNA design and use can be found on the internet at the following addresses:

katahdin.cshl.org:9331/RNAi/docs/13seRI-BamHI_Strategy.pdf and

katahdin.cshl.org:9331 /RNAi/docs/Web_version_of_PCR_strategyl .pdf.

The expression constructs are any constructs suitable for use in the appropriate expression system and include, but are not limited to retroviral vectors, linear expression cassettes, plasmids and viral or virally-derived vectors, as known in the art. Such expression constructs can include one or more inducible promoters, RNA Pol III promoter systems such as U6 snRNA promoters or H1 RNA polymerase III promoters, or other promoters known in the art. The constructs can include one or both strands of the siRNA. Expression constructs expressing both strands can also include loop structures linking both strands, or each strand can be separately transcribed from separate promoters within the same construct. Each strand can also be transcribed from a separate expression construct. Tuschl (2002), supra).

The pharmaceutical compositions can be included in a container, pack, or dispenser together with instructions for administration.

### Methods of Treatment

The present invention provides for both prophylactic and therapeutic methods of treating a subject at risk of (or susceptible to) a disorder or having a disorder associated with aberrant or unwanted TEF expression or activity, e.g., CDK9, CycT1, Spt4, Spt5, or Spt6 activity. As used herein, the term “treatment” is defined as the application or administration of the siRNA compositions of the present invention to an individual, e.g., a patient or subject, or application or administration of a therapeutic composition including the siRNA compositions to an isolated tissue or cell line from an individual who has a disease, a symptom of a disease, or a predisposition toward a disease, with the purpose to cure, heal, alleviate, relieve, alter, remedy, ameliorate, improve, or affect the disease, the symptoms of disease, or the predisposition toward disease. The treatment can include administering siRNAs to one or more target sites on one or both of the P-TEFb subunits, e.g., CDK9 or CycT1, to one or more target sites on one or both of the DSIF subunits, e.g., Spt5 or Spt4, or to target sites on Spt6, as well as siRNAs to other TEFs. The mixture of different siRNAs can be administered together or sequentially, and the mixture can be varied over time.

With regards to both prophylactic and therapeutic methods of treatment, such treatments can be specifically tailored or modified, based on knowledge obtained from the field of genomics, particularly genomics technologies such as gene sequencing, statistical genetics, and gene expression analysis, as applied to a patient's genes. Thus, another aspect of the invention provides methods for tailoring an individual's prophylactic or therapeutic treatment with the siRNA compositions of the present invention according to that individual's genotype; e.g., by determining the exact sequence of the patient's CDK9, CycT1, Spt4, Spt5, and/or Spt6, and designing, using the present methods, an siRNA molecule customized for that patient. This allows a clinician or physician to tailor prophylactic or therapeutic treatments to patients to enhance the effectiveness or efficacy of the present methods.

Also with regards to both prophylactic and therapeutic methods of treatment, such treatments can be specifically tailored or modified, based on knowledge obtained from the field of pharmacogenomics. “Pharmacogenomics,” as used herein, refers to the application of genomics technologies such as gene sequencing, statistical genetics, and gene expression analysis to drugs in clinical development and on the market. More specifically, the term refers to the study of how a patient's genes determine his or her response to a drug (e.g., a patient's “drug response phenotype,” or “drug response genotype.”) Thus, another aspect of the invention provides methods for tailoring an individual's prophylactic or therapeutic treatment with the siRNA compositions of the present invention according to that individual's drug response genotype. Pharmacogenomics allows a clinician or physician to target prophylactic or therapeutic treatments to patients who will most benefit from the treatment and to avoid treatment of patients who will experience toxic drug-related side effects.

In one aspect, the invention provides a method for treating a subject having a disease, disorder, or condition associated with an aberrant or unwanted TEF expression or activity, e.g. CDK9, CycT1, Spt4, Spt5, or Spt6 expression or activity, by administering to the subject a composition including a CDK9, CycT1, Spt4, Spt5, and/or Spt6 siRNA. Subjects having a disease which is caused or contributed to by aberrant or unwanted CDK9, CycT1, Spt4, Spt5, or Spt6 expression or activity can be identified by, for example, any or a combination of diagnostic or prognostic assays known in the art or as described herein. Administration of a composition including a CDK9, CycT1, Spt4, Spt5, or Spt6 siRNA can occur prior to the manifestation of symptoms characteristic of the CDK9, CycT1, Spt4, Spt5, or Spt6 aberrance, such that the disease, disorder, or condition is treated or inhibited.

In one aspect, the invention provides a method for preventing in a subject, a disease or condition associated with an aberrant or unwanted CDK9, CycT1, Spt4, Spt5, or Spt6 expression or activity, by administering to the subject a composition including a CDK9, CycT1, Spt4, Spt5, or Spt6 siRNA. Subjects at risk for a disorder caused or contributed to by aberrant or unwanted CDK9, CycT1, Spt4, Spt5, or Spt6 expression or activity can be identified by, for example, any or a combination of diagnostic or prognostic assays known in the art or as described herein. Administration of a prophylactic agent can occur prior to the manifestation of symptoms characteristic of the CDK9, CycT1, Spt4, Spt5, or Spt6 aberrance, such that a disease or disorder is prevented or, alternatively, delayed in its progression.

The siRNA compositions of the present invention can also be used to control one or more cellular proliferative and/or differentiative disorders or viral diseases.

Examples of cellular proliferative and/or differentiative disorders include cancer, e.g., carcinoma, sarcoma, metastatic disorders, or hematopoietic neoplastic disorders, e.g., leukemias, as well as proliferative skin disorders, e.g., psoriasis or hyperkeratosis. A metastatic tumor can arise from a multitude of primary tumor types, including, but not limited to, those of prostate, colon, lung, breast and liver origin.

As used herein, the terms “cancer,” “hyperproliferative,” and “neoplastic” refer to cells having the capacity for autonomous growth, i.e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness, as long as they are controlled by P-TEFb (CDK9/CycT1), DSIF (Spt4/Spt5) or Spt6. “Pathologic hyperproliferative” cells occur in disease states characterized by malignant tumor growth. Examples of non-pathologic hyperproliferative cells include proliferation of cells associated with wound repair.

The terms “cancer” or “neoplasms” include malignancies of the various organ systems, such as the lung, breast, thyroid, lymphoid, gastrointestinal, and genitourinary tract, as well as adenocarcinomas which include malignancies such as most colon cancers, renal-cell carcinoma, prostate cancer and/or testicular tumors, non-small cell carcinoma of the lung, cancer of the small intestine and cancer of the esophagus, carcinomas, sarcomas, metastatic disorders or hematopoietic neoplastic disorders, e.g., leukemias, as well as proliferative skin disorders, e.g., psoriasis or hyperkeratosis. Metastatic tumors can arise from a multitude of primary tumor types, including but not limited to those of prostate, colon, lung, breast and liver origin.

The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon, and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is art recognized and refers to malignant tumors of mesenchymal derivation.

Additional examples of proliferative and/or differentiative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. For example, the diseases can arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (reviewed in Vaickus et al., Crit. Rev. in Oncol./Hemotol. 11:267-97 (1991)); lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Stemberg disease. Other myeloproliferative disorders include polycythemia vera, myelofibrosis, chronic myelogenous (myelocytic) leukemia, and primary thrombocythaemia, as well as acute leukemia, especially erythroleukemia, and paroxysmal nocturnal haemoglobinuria.

Other examples of proliferative and/or differentiative disorders include skin disorders. The skin disorder may involve the aberrant activity of a cell or a group of cells or layers in the dermal, epidermal, or hypodermal layer, or an abnormality in the dermal-epidermal junction. For example, the skin disorder may involve aberrant activity of keratinocytes (e.g., hyperproliferative basal and immediately suprabasal keratinocytes), melanocytes, Langerhans cells, Merkel cells, immune cell, and other cells found in one or more of the epidermal layers, e.g., the stratum basale (stratum germinativum), stratum spinosum, stratum granulosum, stratum lucidum or stratum corneum. In other embodiments, the disorder may involve aberrant activity of a dermal cell, e.g., a dermal endothelial, fibroblast, immune cell (e.g., mast cell or macrophage) found in a dermal layer, e.g., the papillary layer or the reticular layer.

Examples of skin disorders include psoriasis, psoriatic arthritis, dermatitis (eczema), e.g., exfoliative dermatitis or atopic dermatitis, pityriasis rubra pilaris, pityriasis rosacea, parapsoriasis, pityriasis lichenoiders, lichen planus, lichen nitidus, ichthyosiform dermatosis, keratodermas, dermatosis, alopecia areata, pyoderma gangrenosum, vitiligo, pemphigoid (e.g., ocular cicatricial pemphigoid or bullous pemphigoid), urticaria, prokeratosis, rheumatoid arthritis that involves hyperproliferation and inflammation of epithelial-related cells lining the joint capsule; dermatitises such as seborrheic dermatitis and solar dermatitis; keratoses such as seborrheic keratosis, senile keratosis, actinic keratosis. photo-induced keratosis, and keratosis follicularis; acne vulgaris; keloids and prophylaxis against keloid formation; nevi; warts including verruca, condyloma or condyloma acuminatum, and human papilloma viral (HPV) infections such as venereal warts; leukoplakia; lichen planus; and keratitis. The skin disorder can be dermatitis, e.g., atopic dermatitis or allergic dermatitis, or psoriasis.

In some embodiments, the disorder is psoriasis. The term “psoriasis” is intended to have its medical meaning, namely, a disease which afflicts primarily the skin and produces raised, thickened, scaling, nonscarring lesions. The lesions are usually sharply demarcated erythematous papules covered with overlapping shiny scales. The scales are typically silvery or slightly opalescent. Involvement of the nails frequently occurs resulting in pitting, separation of the nail, thickening and discoloration. Psoriasis is sometimes associated with arthritis, and it may be crippling. Hyperproliferation of keratinocytes is a key feature of psoriatic epidermal hyperplasia along with epidermal inflammation and reduced differentiation of keratinocytes. Multiple mechanisms have been invoked to explain the keratinocyte hyperproliferation that characterizes psoriasis. Disordered cellular immunity has also been implicated in the pathogenesis of psoriasis. Examples of psoriatic disorders include chronic stationary psoriasis, psoriasis vulgaris, eruptive (gluttate) psoriasis, psoriatic erythroderma, generalized pustular psoriasis (Von Zumbusch), annular pustular psoriasis, and localized pustular psoriasis.

Additionally, TEF molecules, e.g. CDK9, CycT1, Spt4, Spt5, and/or Spt6 may play an important role in the etiology of certain viral diseases, including, but not limited to, Human Immunodeficiency Virus (HIV), Hepatitis B, Hepatitis C, and Herpes Simplex Virus (HSV). P-TEFb siRNA compositions can be used to treat viral diseases, and in the treatment of viral infected tissue or virus-associated tissue fibrosis. In particular, as described herein, TEF, e.g. CDK9, CycT1, Spt4, Spt5, and/or Spt6, siRNA compositions can be used to treat HIV infections. Also, TEF modulators can be used in the treatment and/or diagnosis of virus-associated carcinoma, including hepatocellular cancer.

The present invention has a number of advantages. One advantage is that, because the siRNA molecules are targeted to non-viral host genes, there is less concern about mutation, which is of major concern with viral target genes given the propensity of HIV to mutate. In addition, the siRNA molecules can be tailored to the individual patient's genome, so that the effectiveness of the treatment can be optimized-regardless of the amount of genetic variation in the population. In addition, the temporal length of the silencing effect on a TEF, e.g., CDK9, CycT1, Spt4, Spt5, and/or Spt6 can be manipulated to last for a clinically desirable length of time; for instance, use of exogenous siRNAs, e.g., synthetic siRNAs, results in a shorter-lived effect, while use of plasmids or other delivery vectors for expression of the dsRNA in vivo allows for longer-term effects. This allows the clinician to fine tune the treatment, giving the clinician the ability to tailor treatment to the needs of the individual patient; for example, the treatment can be administered every day for any number of days, once a week, twice a week, etcetera, allowing the clinician to monitor the status of the patient and adjust the treatment regime as needed. The treatment can be stopped at any time with no residual effects. The compositions of the present invention can also be administered a number of different ways, including but not limited to targeted transfection (allowing targeting of treatment to a tumor site, for example); injection, e.g., subcutaneous, intradermal, or intraperitoneal; intravenous administration; by needle-free methods or any of the methods described herein. Furthermore, unlike standard cancer treatments, the compositions of the present invention are less toxic and thus less likely to kill normal, healthy cells.

### EXAMPLES

The following materials, methods, and examples are illustrative only and not intended to be limiting.

### Experimental Procedures for Examples 1 - 18

siRNA Preparation

Design of siRNAs against CDK9/CycT1

The targeted region in the mRNA, and hence the sequence of CycT1 or CDK9-specific siRNA duplexes was designed following the guidelines provided by Dharmacon (Lafayette, Colo.). Briefly, starting 100 bases downstream of the start codon, the first AA dimer was located and the next 19 nucleotides were then recorded following the AA dimer. Criteria were set such that the guanosine and cytidine content (G/C content) of the AA-N19 21 base-sequence must be less than 70% and greater than 30%. The search continued downstream until the conditions were met. The 21 -mer sequence was subjected to a BLAST search against the human genome/NCBI EST library to ensure only the desired gene was targeted. The siRNA sequence targeting hCycT1 was from position 347-367 relative to the start codon. The siRNA sequence targeting CDK9 was from position 258-278 relative to the start codon. siRNA sequences used in our experiments were: hCycT1 ds (5′-UCCCUUCCUGAUACUAGAAdTdT-3′) (SEQ ID NO:3); hCycT1 mm (5′-UCCCUUCCGUAUACUAGAAdTdT-3′) (SEQ ID NO:4); CDK9 ds (5′-CCAAAGCUUCCCCCUAUAAdTdT-3′) (SEQ ID NO:5); CDK9 mm (5′-CCAAAGCUCUCCCCUAUAAdTdT-3′) (SEQ ID NO:6); CDK7 ds (5′-UUGGUCUCCUUGAUGCUUUdTdT-3′) (SEQ ID NO:17); Tat ds (5′-GAAACGUAGACAGCGCAGAdTdT-3′) (SEQ ID NO:18); GFP ds (5′-GCAGCACGACUUCUUCAAGdTdT-3′) (SEQ ID NO: 19); and RFP ds (5′-GUGGGAGCGCGUGAUGAACdTdT-3′) (SEQ ID NO:20). Underlined residues represent the mismatched sequence to their targets.

hCycT1 contains an amino-terminal cyclin box motif (amino acids 1-298) that is conserved in the cyclin type protein family, a putative coiled-coil motif (amino acids 379-430) and a histidine-rich motif (amino acids 506-530). The hCycT1 sequence containing amino acids 1-303 is sufficient to form complexes with Tat-TAR and CDK9, as CDK9 binds to the cyclin box (amino acids 1-250) of CycT1. A Tat:TAR recognition motif (TRM) in the hCycT1 sequence that spans amino acids 251-272 is necessary for forming complex with Tat and TAR. Residues 252-260 of hCycT1 have been demonstrated to interact with the TAR RNA loop, suggesting that amino acids 261-272 are involved in interaction with Tat core domain. A critical cysteine (amino acids 261) has been identified as a absolutely requiring residue for the Tat and hCycT1 interaction. The targeted region in the mRNA and hence the sequence of hCycT1-specific siRNA duplexes can be designed targeting to the Cyclin box region or the region for Tat-TAR interaction. Using the guidelines provided by Dharmacon (Lafayette, Colo.) as discussed above, other potential siRNA target sequences include the following: relative to the start codon, the siRNA sequences targeting hCycT1 can be from position 238-278, 502-522, 758-778, 769-789 etc. Based on the guidelines of Dharmacon as discussed above, additional siRNA sequences suitable for targeting CDK9 can be from position 220-240, 258-278, 379-399relative to the start codon.

SiRNA Synthesis and Maintenance

21-nt ds RNAs were chemically synthesized as 2′ bis(acetoxyethoxy)-methyl ether-protected oligos by Dharmacon (Lafayette, Colo.). Synthetic oligonucleotides were deprotected, annealed and purified according to the manufacturer's recommendation. Successful duplex formation was confirmed by 20% non-denaturing polyacrylamide gel electrophoresis (PAGE). All siRNAs were stored in DEPC (0.1% diethyl pyrocarbonate)-treated water at −80° C.

Culture and Transfection of Cells

HeLa cells were maintained at 37° C. in Dulbecco's modified Eagle's medium (DMEM, Invitrogen) supplemented with 10% fetal bovine serum (FBS), 100 unit/ml penicillin and 100 μg/ml streptomycin (Invitrogen). Magi (multinucleate activation of galactosidase indicator) cells harboring the endogenous HIV LTR-β-galactosidase gene were maintained at 37° C. in DMEM, supplemented with 10% FBS, 0.2 mg/ml Geneticin (G418) and 0.1 mg/ml hygromycin B (Roche Molecular Biochemicals). T47D (HTB 133) cells were purchased from American Type Culture Collection (ATCC, Manassas, Va.) and cultured in RPMI 1640 medium (Invitrogen), supplemented with 10% FBS and 0.2 units/ml bovine insulin (Invitrogen). Cells were regularly passaged at sub-confluence and plated at 70% confluency 16 hours before transfection. Lipofectamine (Invitrogen)-mediated transient cotransfections of reporter plasmids and siRNAs were performed in duplicate 6-well plates (Falcon) as described by the manufacturer for adherent cell lines. A standard transfection mixture containing 100 nM siRNA and 10 μl Lipofectamine™ in 1 ml serum-reduced OPTI-MEM (Invitrogen) was added to each well. Cells were incubated in transfection mixture for 6 hours and further cultured in antibiotic-free DMEM. For Western blot analysis at various time intervals, the transfected cells were washed twice with phosphate buffered saline (PBS, Invitrogen), flash frozen in liquid nitrogen, and stored at −80° C. for analysis. For in vivo assays of Tat-mediated transactivation at 48 hours post transfection, Magi cells were subjected to β-galactosidase staining directly or flash frozen in liquid nitrogen and stored at −80° C. for β-galactosidase assay as described below.

siRNA Delivery for Longer-Term Expression

Synthetic siRNAs can be delivered into cells by cationic liposome transfection and electroporation. However, these exogenous siRNA only show short term persistence of silencing effect (4˜5 days). Several strategies for expressing siRNA duplexes within cells from recombinant DNA constructs allow longer-term target gene suppression in cells, including mammalian Pol III promoter systems (e.g., H1 or U6/snRNA promoter systems (Tuschl (2002), supra) capable of expressing functional double-stranded siRNAs; (Bagella et al. (1998) J. Cell. Physiol. 177, 206-213; Lee et al. (2002), supra; Miyagishi et al. (2002), supra; Paul et al. (2002), supra; Yu et al. (2002), supra; Sui et al. (2002) supra). Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence under the control of T7 promoter also make functional siRNs when cotransfected into the cells with a vector expression T7 RNA polymerase (Jacque (2002), supra).

It has been shown that animal cells express a range of noncoding RNAs of approximately 22 nucleotides termed micro RNA (miRNAs) and can regulate gene expression at the post transcriptional or translational level during animal development. One common features of miRNAs is that they are all excised from an approximately 70 nucleotide precursor RNA stem-loop, probably by Dicer, an RNase III-type enzyme, or a homolog thereof. By substituting the stem sequences of the miRNA precursor with miRNA sequence complementary to the target mRNA, a vector construct which expresses the novel miRNA can be used to produce siRNAs to initiate RNAi against specific mRNA targets in mammalian cells (Zeng (2002), supra). When expressed by DNA vectors containing polymerase III promoters, micro-RNA designed hairpin are active on silencing gene expression (McManus (2002), supra). Viral-mediated delivery mechanism can also be used to induce specific silencing of targeted genes through expression of siRNA, for example by generating recombinant adenoviruses harboring siRNA under RNA Pol II promoter transcription control (Xia et al. (2002), supra). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. Id. In an animal model, whole-embryo electroporation can efficiently deliver synthetic siRNA into post-implantation mouse embryos (Calegari et al. (2002), Proc. Natl. Acad. Sci. U S A, 99(22), 14236-40). In adult mice, efficient delivery of siRNA can be accomplished by “high-pressure” delivery technique, a rapid injection (within 5 seconds) of a large volume of siRNA containing solution into animal via the tail vein (Liu (1999), supra; McCaffrey (2002), supra; Lewis (2002), Nature Genetics 32, 107-108.). Nanoparticles and liposomes can also be used to deliver siRNA into animals.

Western Blotting

Cells treated with siRNA were harvested as described above and lysed in ice-cold reporter lysis buffer (Promega) containing protease inhibitor (complete, EDTA-free, 1 tablet/10 ml buffer, Roche Molecular Biochemicals). After clearing the resulting lysates by centrifugation, protein in clear lysates was quantified by Dc protein assay kit (Bio-Rad). Proteins in 60 μg of total cell lysate were resolved by 10% SDS-PAGE, transferred onto a polyvinylidene difluoride membrane (PVDF, Bio-Rad), and immuno-blotted with antibodies against hCycT1 and CDK9 (Santa Cruz). Protein content was visualized with a BM Chemiluminescence Blotting Kit (Roche Molecular Biochemicals). The blots were exposed to x-ray film (Kodak MR-1) for various times (30 s to 5 min).

RT-PCR for Amplification of hCycT1 and CDK9 mRNA

Total cellular mRNA was prepared from HeLa cells with or without hCycT1/CDK9 siRNA treatment using a Qiagen RNA mini kit, followed by an oligotex mRNA mini kit (Qiagen). RT-PCR was performed using a SuperScript One-Step RT-PCR kit with platinum Taq (Invitrogen) and 40 cycles of amplification. Each RT-PCR reaction included 100 ng total cellular mRNA, gene-specific primer sets for hCycT1 and CDK9 amplification (0.5 μM for each primer), 200 μM dNTP, 1.2 mM MgSO₄ and 1U of RT/platinum Taq mix. Primer sets for hCycT1 produced 2178 bp products, while CDK9 primer sets produced 1116 bp products. RT-PCR products were resolved in 1% agarose gel and viewed by ethidium bromide staining.

RT-PCR for amplification of BCSG1 mRNA

Total cellular mRNA was prepared from T47D cells with or without hCycT1 siRNA treatment, and RT-PCR was performed as described above, except that BCSG1 gene-specific primer sets (0.5 mM for each primer) were used. Glyceraldehyde 3-phosphate dehydrogenase (GAPDH) was used as a reference gene in the RT-PCR reaction. Primer sets for BCSG1 produced 384 bp products, while GAPDH primer sets amplify the coding sequence of GAPDH from 539 to 759 and produced 221 bp products.

Plasmids Harboring the HIV-1 Tat Sequence

pTat-RFP plasmids were constructed by fusing the DNA sequence of HIV-1 Tat with DNA sequences of DsRed1-N1, harboring coral (Discosoma spp.)-derived red fluorescent protein (RFP), per the manufacturer's recommendation (Clontech). Cytomegalovirus promoter control drove the expression of Tat-RFP fusion proteins, which were easily visualized in living cells by fluorescence microscopy (Zeiss). Expression of Tat-RFP fusion proteins was also quantified by directly exciting the RPF fluorophore in clear cell lysates and measuring fluorescence, as described below.

β-Galactosidase Staining of Cells

Magi cells were transfected with Tat-containing plasmids in the absence or presence of siRNAs. At 48 h post transfection, cells were washed twice with PBS and fixed 5 min in fixative (1% formaldehyde and 0.2% glutaraldehyde in PBS) at room temperature. After washing twice with PBS, cells were covered with staining solution (PBS containing 4 mM potassium ferrocyanide, 4mM potassium ferricyanide, 2 mM MgCl₂ and 0.4 mg/ml X-gal [Promega]) and incubated at 37° C. for exactly 50 min. Plates were washed twice with PBS. Cell counts represent number of β-galactosidase positive (blue) cells per 100-power field.

β-Galactosidase Enzyme Assay

Magi cells were transfected with Tat-containing plasmids in the absence or presence of siRNAs. At 48 hours post transfection, cells were harvested and clear cell lysates were prepared and quantified as described above. Total cell lysate (120 μg) in reporter lysis buffer (150 μl) was subjected to standard β-galactosidase assay by adding 150 μl 2×β-galactosidase assay buffer (Promega) and incubating at 37° C. for 30 min. The reactions were stopped by adding 500 μl 1M sodium carbonate and briefly vortexing. Absorbance was read immediately at 420 nm. The same amount of cell lysate was subjected to fluorescence measurements on a PTI (Photon Technology International) fluorescence spectrophotometer, with slit widths set at 4 nm for both excitation and emission wavelengths. All experiments were carried out at room temperature. Fluorescence of Tat-RFP in the cell lysate was detected by exciting at 568 nm and recording the emission spectrum from 588 nm to 650 mn; the spectrum peak at 583 nm represents the maximum fluorescence intensity of Tat-RFP. Tat transactivation was determined by calculating the ratio of β-galactosidase activity (absorbance at 420 nm) of the pTat-RFP transfected cells to that of cells without pTat-RFP plasmid treatment. The inhibitory effect of siRNA treatment was determined by normalizing Tat-transactivation activity to the amount of Tat-RFP protein (represented by RFP fluorescence intensity) in the presence and absence of siRNA.

In vivo Fluorescence Analysis

pEGFP-C 1 reporter plasmids (1 μg) and siRNA (100 nM) were cotransfected into HeLa cells by Lipofectamine™ as described above, except that cells were cultured on 35 mm plates with glass bottoms (MatTek Corporation, Ashland Mass.) instead of standard 6-well plates. Fluorescence in living cells was visualized 50 hours post transfection by conventional fluorescence microscopy (Zeiss). For GFP fluorescence detection, FITC filter was used.

GeneChip® Experiments

Total cellular mRNA was prepared from HeLa cells with or without hCycT1/CDK9 ds siRNA treatment using a Qiagen RNA mini kit followed by oligotex mRNA mini kit. Double-stranded cDNAs were synthesized from 2 μg total mRNA using the Superscript Choice System for cDNA synthesis (Invitrogen) with the T7-(dT)24 primer following the manufacturer's recommendations. cDNAs were cleaned up by phase lock gel (PLG) (Brinkman Instrument)-phenol/chloroform extraction and concentrated by ethanol precipitation. Biotin-labeled cRNA was synthesized from cDNA by in vitro transcription using the Bioarray HighYield RNA transcript Labeling Kit (Affymetrix) following vendor's recommendation. In vitro transcription products were cleaned up using RNeasy spin columns (Qiagen) and fragmented into 35-200 base units by metal-induced hydrolysis in fragmentation buffer (40 mM Tris-acetate, pH 8.1, 100 mM KOAc, 30 mM MgOAc). Fragmented cRNA was then subjected to Affymetrix Human Genome U133A and U133B GeneChip® sets in hybridization buffer (100 mM MES, 1M NaCl, 20 mM EDTA, 0.01% Tween-20). All hybridization, washing, staining and scanning procedures were performed in the UMASS Medical School Genomics Core Facility following the manufacturer's protocols. GeneChip® images were analyzed with Affymetrix Microarray Suite V5.0 and Affymetrix Data Mining Tool V3.0. Signal intensities of all probe sets were scaled to a target value of 150. Results of Detection Call, Change Call and Signal Log Ratio were obtained by applying the default parameters to statistical algorithms for both absolute and comparison analyses.

Magi Infectivity Assay (HIV-1 Replication Assay)

HeLa-CD4-LTR/β-galactosidase indicator (Magi) cells (Kimpton and Emerman (1992), J. Virol., 66, 2232-2239) were plated in 24-well plates (7.5×10⁵ cells per well) and transfected with siRNAs as previously described (Jacque et al. (2002), Nature, 418, 435-438). siRNA (60 pmol) was transfected into cells using oligofectamine (2 μl, Invitrogen) for 3 hours in serum-free DMEM (GIBCO). Cells were rinsed twice and top-layered in 500 μl of DMEM-10% FBS. Sixteen hours after transfection, cells were trypsinized and seeded in 96-well microtiter plates (4×10⁴ cells per well), incubated 3 hours and infected. HIV-1 virions (normalized to RT activity in cpm) were added in doubling dilutions to duplicate wells. Thirty-six hours post infection, cells were harvested to quantify β-galactosidase activity.

Soft Agar Assay

Anchorage-independent growth was carried out in 24-well culture plates (Falcon). T47D cells were treated with siRNAs in a 60 mm plate and 24 hours later cells were split into a 24-well plate. The bottom layer consisted of 375 ml of RPMI medium containing 10% FBS and 0.5% agar, and the top layer contain 10% FBS and 0.33% agar plus 2.5×104 cells. At 99 hours post transfection, three fields in each duplicate well (total six fields) were randomly selected and viewed under a microscope at 200×. The number of colonies containing 5-7 and 8-10 cells were counted separately and averaged.

Immunoprecipitation

Protein extracts were prepared by sonicating cells in RIPA buffer (20 mM Tris-HCl, pH 8.0, 0.5% Nonidet P-40, 1% Triton X-100, and 150 mM KCl, 5 mM DTT) containing protease inhibitor (Roche, 1 tablet/10 mL) and recombinant RNAsin RNase inhibitor (Promega, 1U/μl). Cell lysates (300 μg of protein) were immunoprecipitated with 3 μg anti-CDK9 antibodies (Santa Cruz) which had been adsorbed to protein G-Sepharose beads (Amersham Pharmacia Biotech) in RIPA buffer during an overnight incubation at 4° C. The beads were than washed three times with 300 μl of RIP A buffer containing 0.5% NP-40 and 1 M KCl and once with 300 μl of RIP A buffer. The beads were resuspended in 200 μl of RIPA buffer and split into two equal aliquots. One of the aliquots was treated with 10 units of RNase A (Amersham Pharmacia Biotech) for 15 min at 30° C. RNaseA treatment was stopped by washing the beads with 300 μl RIPA buffer for three times. The second aliquot was not treated with RNase A. The beads treated or non-treated with RNase A were then split into three equal aliquots: one for silver stain analysis; one for western analysis; and the other for kinase activity analysis.

Kinase Activity Analysis of Immunoprecipitates

Kinase assays were performed on anti-CDK9 immunoprecipitates (with or without RNase A treatment as described above) at 37° C. for 1 h in 20 mM Tris-HCl (pH 8.0), 5 mM MgCl₂, 60 mM NaCl and 10 μM ATP and [γ-³²P]-ATP in a total volume of 45 ml. The reaction was terminated by the addition of 15 ml of 4×Laemmli sample buffer. The phosphorylated protein was visualized by autoradiography after electrophoresis in a 10% SDS-polyacrylamide gel. The incorporation of ³²P was quantified by phosphorimager analysis.

### Example 1

### Specific Silencing of P-TEFb Expression bv siRNA in HeLa Cells

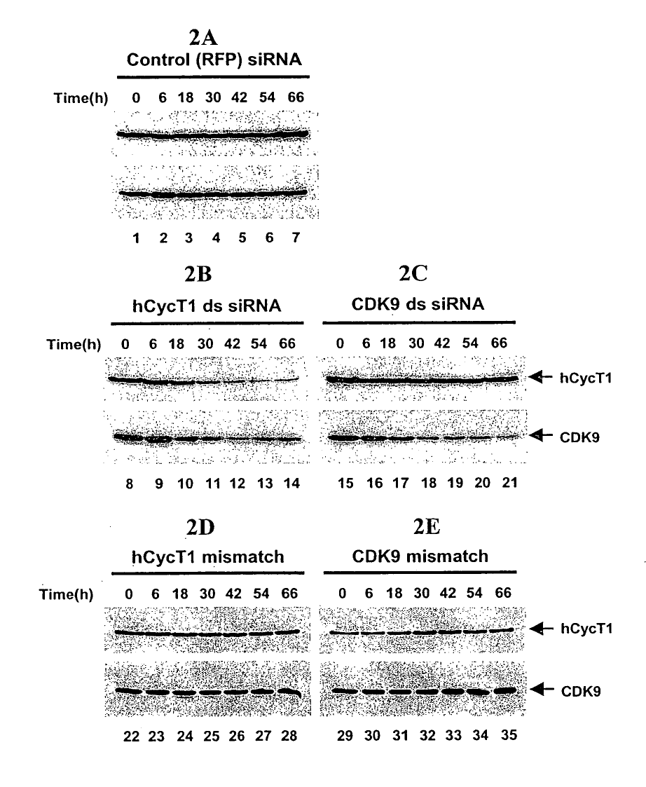

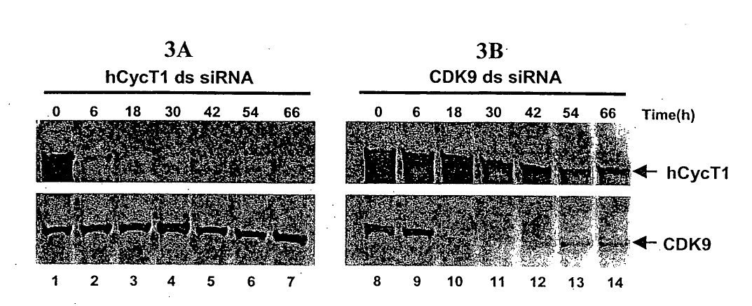

RNAi was used to inhibit hCycT1 and CDK9 expression in cultured human (HeLa) cell lines. The short interfering RNA (siRNA) sequence targeting hCycT1 was from position 347 to 367 relative to the start codon, and the CDK9 siRNA sequence was from position 258 to 278 relative to the start codon. Using lipofectamine, HeLa cells were transfected with hCycT1 or CDK9 siRNA duplex, targeting either hCycT1 or CDK9. To analyze RNAi effects, lysates were prepared from siRNA duplex-treated cells at various times after transfection. Western blot experiments were carried out using anti-hCycT1 and anti-CDK9 antibodies (see FIGS. 2A-2E). Briefly, HeLa cells were transfected with double-stranded (ds) siRNAs targeting RFP (control, FIG. 2A, lanes 1-7), hCycT1 (FIG. 2B, lanes 8-14), or CDK9 (FIG. 2C, lanes 15-21). Cells were also transfected with mutant siRNAs (hCycT1 mismatch [FIG. 2D, lanes 22-28] or CDK9 mismatch [FIG. 2E, lanes 29-35]) having 2 nucleotide mismatches between the target mRNA and the antisense strand of siRNA at the hypothetical cleavage site of the mRNA. Cells were harvested at various times post transfection, their protein content resolved on 10% SDS-PAGE, transferred onto PVDF membranes, and immunoblotted with antibodies against hCycT1 and CDK9. Analysis of immunoblotting experiments reveals that the siRNA targeting hCycT1 inhibited hCycT1 protein expression (FIG. 2B, lanes 8-14, upper panel). siRNA targeting CDK9 was similarly specific against CDK9 expression (FIG. 2C, lanes 15-21, lower panel). This RNAi effect depended on the presence of a 21-nt duplex siRNA harboring a sequence complementary to the target mRNA, but not on single stranded antisense strand siRNAs (data not shown) nor on an unrelated control siRNA, which targeted a coral (Discosoma spp.)-derived red fluorescent protein (RFP) (FIG. 2A, lanes 1-7). As a specificity control, cells were also transfected with mutant siRNAs (mismatched siRNA) of hCycT1 or CDK9, which have two nucleotide mismatches between the target mRNA and the antisense strand of siRNA at the putative cleavage site of the mRNA. Mutant siRNAs showed no interference activity (FIGS. 2D and 2E, lanes 22-28 and 29-35, respectively), indicating the specificity of the RNAi effect. Thus, the siRNAs of the present invention specifically silence the subunits of P-TEFb in HeLa cells.

### Example 2

### Kinetics of P-TEFb RNA Interference in HeLa Cells

RNA interference is a highly efficient process because a few dsRNA molecules are sufficient to inactivate a continuously transcribed target mRNA for long periods of time. Experiments have shown in plants and worms (Cogoni, C., and Macino, G. (1999). Gene silencing in Neurospora crassa requires a protein homologous to RNA-dependent RNA polymerase. Nature 399, 166-169; Dalmay et al. (2000), Cell, 101, 543-553; Grishok et al. (2000), Science, 287, 2494-2497) that this inactivation can spread throughout the organism and is often heritable to the next generation. Mutations in genes encoding proteins related to RNA-dependent RNA polymerase (RdRP) affect RNAi-type processes in Neurospora, Caenorhabditis elegans and plants (Cogoni, C., and Macino, G., 1999, supra; Dalmay et al., 2000, supra; Lipardi et al. (2001). Cell, 107, 297-307; Mourrain et al. (2000), Cell, 101, 533-542; Smardon et al. (2000), Curr. Biol., 10, 169-178), and the involvement of RdRP in amplifying RNAi has been postulated (Lipardi et al., 2001, supra).

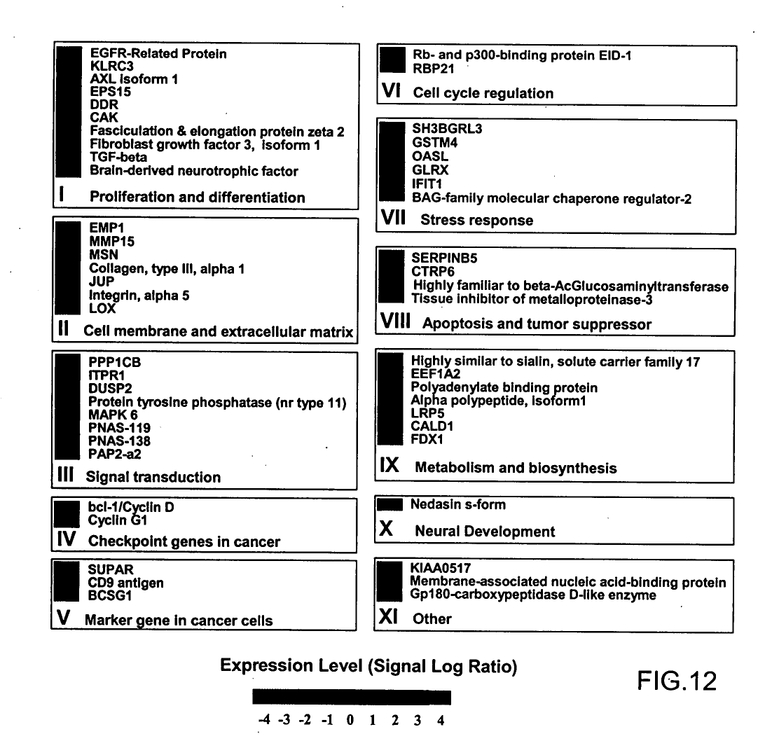

Western blot experiments (FIGS. 2A-E) using anti-hCycT1 and anti-CDK9 antibodies also revealed the kinetics of gene suppression and persistence of RNAi in HeLa cells. Briefly, ds siRNAs were transfected into HeLa cells as described in the Experimental Procedures. Cells were harvested at various times and protein contents were resolved on 10% SDS-PAGE, transferred onto PVDF membranes, and immunoblotted with antibodies against hCycT1 and CDK9. Western blotting of hCycT1 and CDK9 over a 90-hour time course are shown in FIG. 12. Although RNAi can suppress expression of hCycT1 and CDK9 proteins up to 66 hours post transfection, maximum activities were observed at 42-54 h, and inhibition by siRNAs did not persist. After reaching maximal activity at 42-54 hours post transfection, RNA interference started to decrease at 54 h, with protein amount showing gradual recovery to normal levels between 66 to 90 hours (3 to 4 days) post transfection (FIG. 12). Similar phenomena were demonstrated in 293T cells, a human T lymphocyte cell line (data not shown). The recovery of target gene expression indicates that RNAi by exogenous siRNA duplexes lasted comparably to other genes silenced previously in mammalian cells (Surabhi 2002 J. Virol. 76:12963-73) and does not last indefinitely in mammalian cells. These findings suggest that the amplification system driven by RdRP in plants and nematodes may not exist or has very little effect on siRNA-mediated gene silencing in mammalian cells. These data indicate that the effect of siRNA on hCycT1 and CDK9 is transient, which allows for great flexibility in customizing treatment plans and removal of treatment without residual effects.

An intriguing finding revealed by the above analysis of hCycT1 knockdown was that cells transfected with hCycT1 siRNA had significantly down-regulated CDK9 protein levels. The kinetics of CDK9 knockdown by hCycT1 siRNA shows a pattern similar to hCycT1 knockdown (FIG. 2B, lanes 8-14, lower panel). No homologous sequence has been found between the CDK9 mRNA and hCycT1 siRNA used in this assay, suggesting that CDK9 knockdown by hCycT1 siRNA was not due to cleavage of CDK9 mRNA by the RNAi pathway. Two possibilities could explain this unexpected down-regulation. First, hCycT1 knockdown may have affected CDK9 protein stability. If protein-protein contacts between CDK9 and hCycT 1 were involved in forming a stable P-TEFb complex, these hCycT1-CDK9 interactions may be required to stabilize CDK9 in the cell. Second, since P-TEFb is a positive transcription factor, it is possible that the P-TEFb complex is required for transcription of the CDK9 gene. If this was the case, down-regulation of hCycT1 by hCycT1 siRNA would decrease the amount of P-TEFb complexes in the cell available for transcription and in turn down-regulate intracellular CDK9 transcription.

### Example 3

### Specific Silencing of P-TEFb by siRNA at the mRNA Level and Stability of CDK9

To determine the specificity of P-TEFb knockdown by siRNA at the mRNA level and to distinguish the two hypotheses proposed above, RT-PCR was performed to reveal the effect of siRNA on the level of mRNA involved in P-TEFb expression. Briefly, HeLa cells were transfected with hCycT1 ds siRNA (FIG. 3A, lanes 1-7) and CDK9 ds siRNA (FIG. 3B, lanes 8-14), harvested at various times after transfection and mRNAs extracted. One-step RT-PCR was performed, setting the specific primer for hCycT1 and CDK9 amplification. RT-PCR products were resolved in 1% agarose gel and viewed by ethidium bromide staining. As shown in FIG. 3A, transfection of cells with siRNA duplex targeting hCycT1 (hCycT1 ds) significantly reduced hCycT1 expression (lanes 1-7, upper panel), but had no effect on CDK9 mRNA (lanes 1-7, lower panel).

On the other hand, transfection of cells with siRNA duplex targeted to CDK9 (CDK9 ds) significantly interfered with the expression of CDK9, but not hCycT1 (FIG. 3B, lanes 8-14). These results suggested that hCycT1 knockdown did not result in decreased transcription of CDK9 mRNA. The siRNA duplex started to cause an RNAi effect as early as 6-18 hours post transfection and gradually increased with time, peaking at 30 h, and decreased between 54-66 h. The time-dependent effect of siRNA indicates that siRNAs need to be processed or assembled into an active complex with cellular factors for effective RNA interference. A time lag was also seen between the degradation of target mRNA (starting at 6 hours post siRNA transfection, as shown by semi-quantitative RT-PCR in FIGS. 3A-3B) and the half-life of the existing protein expressed by the target gene, because protein levels did not show any down-regulation until 18-30 hours post siRNA transfection (FIGS. 2B-2C). Combined with Western blot analysis (FIG. 2B-2C), semi-quantitative RT-PCR (FIGS. 3A-3B) not only confirms the specific knockdown of P-TEFb by siRNA at the mRNA level, but also suggests that forming a complex with hCycT1 is a prerequisite for maintaining the stability of CDK9 proteins in living cells. Thus, hCycT1 siRNA down-regulated hCycT1 levels by the RNAi pathway, while down-regulating CDK9 levels by promoting its degradation without affecting its gene expression at the mRNA level. This indicates that the use of hCycT1 siRNA, even without added CDK9 siRNA, is able to down regulate both P-TEFb and CDK9 activity.

### Example 4

### hCycT1 and CDK9 Knockdown are Not Lethal to Human Cells

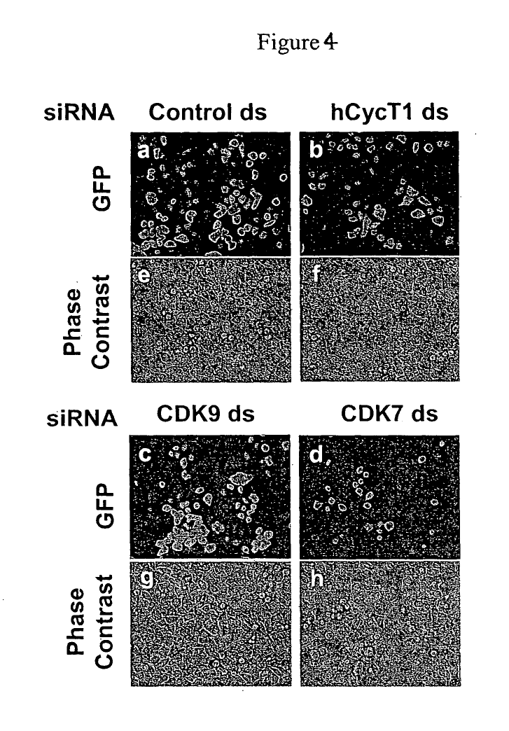

To analyze the viability of cells subjected to P-TEFb gene silencing, a pEGFP-C1 reporter plasmid, harboring enhanced green fluorescent protein [GFP] under the cytomegalovirus (CMV) immediate early promoter, plus hCycT1 and CDK9 siRNAs were co-transfected into HeLa cells using lipofectamine. Briefly, HeLa cells were cotransfected by Lipofectamine™ with pEGFP-C1 reporter (GFP) plasmid and siRNAs. Four siRNA duplexes, including a control duplex targeting RFP (FIG. 4, panels a and e) and three duplexes targeting hCycT1 (FIG. 4, panels b and f), CDK9 (FIG. 4, panels c and g), and CDK7 (FIG. 4, panels d and h), were used in these experiments. Reporter gene expression was monitored at 50 hours post transfection by fluorescence imaging in living cells (FIG. 4, upper panels, a-d). Cellular shape and density were recorded by phase contrast microscopy (lower panels, e-h). Reporter gene (GFP) expression, driven by cytomegalovirus (CMV) immediate early promoter, was monitored in living cells (FIG. 4, upper panels a-d). Cellular morphology and density were monitored by phase contrast microscopy (FIG. 4, lower panels e-h). GFP expression was not affected by hCycT1 or CDK9 knockdown (FIG. 4, compare panels a-c). Cells with P-TEFb knockdown had normal shape and growth rate. At 50 hours post transfection, cell density reached ˜90% to 100% confluency (FIG. 4, compare panels e-g).

For comparison, cells were transfected with siRNA targeting CDK7, a well-characterized kinase required for TFIIH, an essential transcription factor, to phosphorylate the CTD of RNA pol II at the step of promoter clearance during initiation of transcription. Kin28, a protein in Saccharomyces cerevisiae that is equivalent to CDK7 in mammals, is an essential gene product that phosphorylates Ser5 of the CTD YSPTSPS repeat region (Komarnitsky et al. (2000), Genes Dev., 14, 2452-2460; Rodriguez et al. (2000), Mol. Cell. Biol., 20, 104-112; Schroeder et al. (2000), Genes & Dev., 14, 2435-2440) and is required to recruit the mRNA capping enzyme to the transcription machinery (Cho et al. (1997), Genes & Dev., 11, 3319-3326; McCracken et al. (1997), Genes & Dev., 11, 3306-3318; McCracken et al. (1997), Nature, 385, 357-361; Yue et al. (1997), Proc. Natl. Acad. Sci. USA, 94, 12898-12903). CDK7 is a bifunctional enzyme in larger eukaryotes, promoting both CDK activation and transcription (Harper and Elledge. (1998), Genes & Dev., 12, 285-289). As expected, reduction of CDK7 levels by RNAi led to a lower reporter (GFP) expression and an arrest in cellular growth (FIG. 4, panel d). CDK7 knockdown cells were smaller than control cells and showed blebbing (FIG. 4, panel h), indicating that unlike RNAi of P-TEFb, CDK7 gene silencing had an adverse affect on transcription, cell morphology and cell growth.

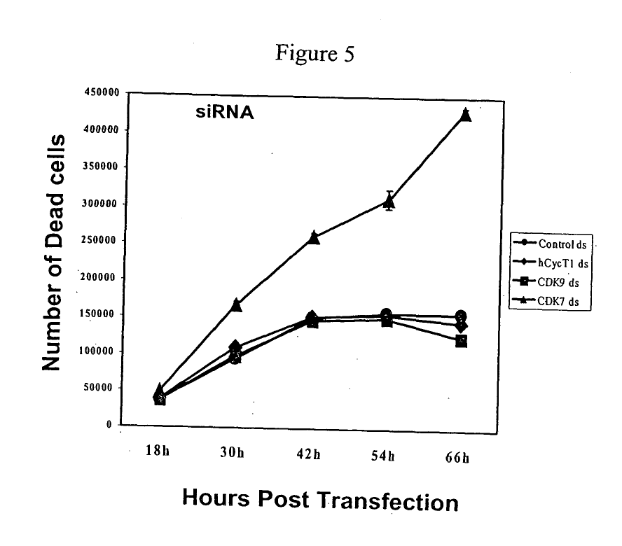

Cellular viability was next analyzed under various siRNA treatments. At various times after transfection, cell viability was assessed by trypan blue exclusion (see below). Briefly, HeLa cells were cotransfected by Lipofectamine™ with pEGFP-C1 reporter (GFP) plasmid and siRNAs (see Experimental Procedures). Four siRNA duplexes, including a control unrelated duplex (FIG. 5, circles) and three duplexes targeting hCycT1 (FIG. 5, diamonds), CDK9 (FIG. 5, squares), and CDK7 (FIG. 5, triangles), were used in these experiments. At various times after transfection, cells floating in the medium were collected and counted in the presence of 0.2% trypan blue (see Experimental Procedures). Cells that took up dye (stained blue) were not viable. Over a 66 hours time course experiment, the rate of cell death in P-TEFb (hCycT1 or CDK9) knockdown cells was comparable to that in control cells with unrelated siRNA treatment, while CDK7 knockdown cells showed a significant increase in cell death (FIG. 5). These results indicate that P-TEFb knockdown is not lethal to human cells, while a much more stringent threshold for CDK7 is required to maintain cell viability and growth.

### Example 5

### hCycT1 and CDK9 RNAi Inhibit HIV-1 Tat Transactivation in Human Cells

A dominant paradigm for Tat up-regulation of HIV gene expression at the level of transcription elongation revolves around the ability of the Tat-TAR RNA complex to bind to P-TEFb and stimulate phosphorylation of the CTD and Spt5, thereby overriding the elongation arrest elicited by DSIF and NELF (Ping and Rana, 2001, supra; Price, 2000, supra). To test whether siRNAs that targeted sequence elements of P-TEFb would specifically block Tat transactivation, Magi cells were cotransfected with the Tat expression construct pTat-RFP and hCycT1 or CDK9 ds siRNA or as controls, antisensehCycT1 or CDK9 siRNA, mutant hCycT1 or CDK9 siRNA, or non-P-TEFb duplex siRNA. Magi, a HeLa cell line harboring a single copy of persistently transfected HIV-1 LTR-β-galactosidase gene, is programmed to express the CD4 receptor and the CCR5 coreceptor for HIV-1, making them a model cell line for measuring HIV replication (Kimpton and Emerman, 1992, supra). It was confirmed that the HIV-1 Tat-RFP fusion protein was expressed under control of the CMV early promoter in all transfected cells by Western blot, using anti-RFP antibody.

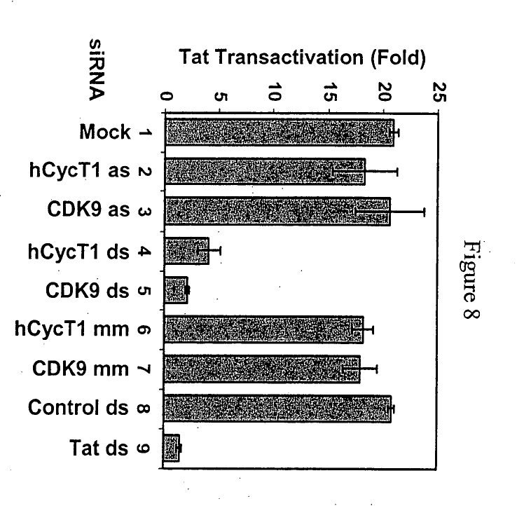

Tat-RFP strongly enhanced β-galactosidase gene expression, which is under control of the HIV-1 LTR promoter in transfected Magi cells. Tat transactivation was determined by calculating the ratio of β-galactosidase activity in pTat-RFP transfected cells to the activity in cells without pTat-RFP treatment. Inhibitory activity was determined by normalizing Tat-transactivation activity to the amount of Tat-RFP protein (represented by RFP fluorescence intensity as described in Experimental Procedures) in the presence and absence of siRNA. Briefly, twenty-four hours after pre-treating Magi cells with siRNA, they were cotransfected with pTat-RFP plasmid and various siRNAs. Cells were harvested 48h post pTat-RFP transfection, and activity of β-galactosidase in clear cell lysates was measured (see Experimental Procedures). Tat-RFP transfection (mock) is shown in FIG. 8, bar 1. Magi cells were cotransfected with ds siRNAs targeting hCycT1 and CDK9 (FIG. 8, bars 4 and 5), with antisense (as) RNA strands (FIG. 8, bars 2 and 3), or mutant (mm) siRNAs (FIG. 8, bars 6 and 7). GFP ds siRNA was used as an unrelated control siRNA (FIG. 8, bar 8), while Tat ds siRNA, targeting the mRNA encoding Tat sequence, was used as a positive control (FIG. 8, bar 9). Means±SD of two experiments are shown. Under standard experimental conditions, Tat-RFP enhanced gene transactivation 20- to 25-fold (FIG. 8, bar 1). This activation was strongly inhibited by cotransfecting host Magi cells with the specific ds siRNAs targeting hCycT1 and CDK9 (FIG. 8, bars 4 and 5), but not with antisense (as) RNA strands (FIG. 8, bars 2 and 3), mutant (mm) siRNAs (FIG. 8, bars 6 and 7), or an unrelated control siRNA (FIG. 8, bar 8).

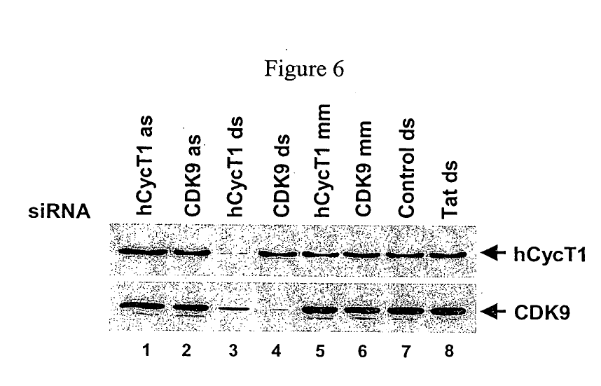

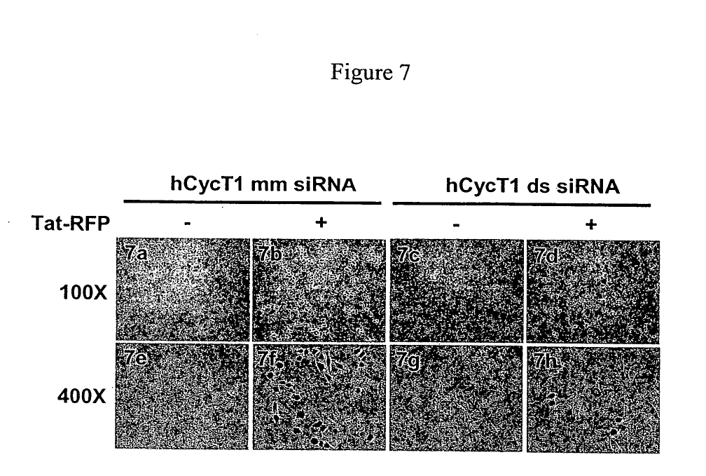

Specific RNA interference with hCycT1 and CDK9 expression in Magi cells was demonstrated by Western blot analysis. Briefly, Magi cells were co-transfected with pTat-RFP plasmid and various siRNAs. Cells were harvested at 48 hours post transfection, resolved on 10% SDS-PAGE, transferred onto PVDF membranes, and immunoblotted with antibodies against hCycT1 (FIG. 6, upper panel) and CDK9 (FIG. 6, lower panel). RNAi activities in Magi cells treated with antisense (as) strands of hCycT1 and CDK9 siRNAs are shown in FIG. 6, lanes 1 and 2, while those of cells treated with ds siRNA targeting hCycT1 and CDK9 are shown in FIG. 6, lanes 3 and 4. RNAi activities in cells treated with mutant hCycT1 siRNA (hCycT1 mm) or mutant CDK9 siRNA (CDK9 mm) are shown in FIG. 6, lanes 5 and 6. GFP ds siRNA was used as an unrelated control (FIG. 6, lane 7), while Tat ds RNAi was used to target mRNA encoding Tat (FIG. 6, lane 8). The inhibition of Tat transactivation correlated well with the knockdown of hCycT1 and CDK9 protein levels by the hCycT1 and CDK9 siRNAs (compare FIG. 8, bars 4 and 5 to FIG. 6, lanes 3 and 4). As shown in FIG. 7, syncytia formation and LTR activation (represented by cells with dark β-galactosidase staining) were reduced in hCycT1 ds siRNA-treated cells (FIG. 7, compare panels d to h and b to f). From these results, it can be concluded that siRNA targeting P-TEFb can inhibit Tat-transactivation in human cells without affecting cellular viability, thus making siRNA targeting P-TEFb an excellent candidate for treatment of patients infected with HIV.

### Example 6

### hCycT1 and CDK9 RNAi Inhibit HIV-1 Infectivity

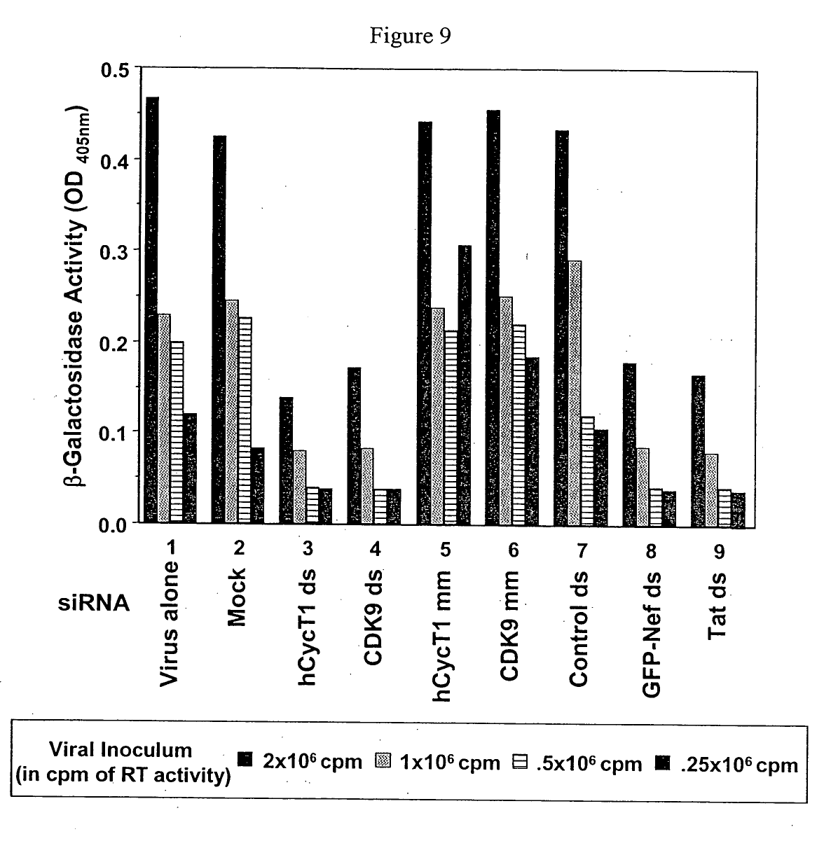

The next question addressed was whether targeting the human P-TEFb complex by RNAi inhibited HIV replication. To investigate this question, HeLa-CD4-LTR/β-galactosidase (Magi) cells were transfected with homologous and mismatched siRNAs directed against hCycT1 or CDK9 and 16 hours later infected the Magi cells with various concentrations of HIV_{NL-GFP}, an infectious molecular clone of HIV-1. HIV-1 Tat-mediated transactivation of the LTR led to β-galactosidase production that was quantified 36 hours post-infection. Briefly, LTR/ [symbol] -galactosidase (Magi) cells transfected with homologous (FIG. 9, ds, bars 3 and 4) and mismatched (FIG. 9, mm, bars 5 and 6) siRNAs directed against CycT1 or CDK9. Cells were also mock transfected without siRNA (FIG. 9, bar 2) or transfected with an unrelated ds siRNA against the RFP sequence (FIG. 9, bar. 7). Sixteen hours later, cells were infected with NL-GFP, an infectious molecular clone of HIV-1. Cells infected with virus and not treated with oligofectamine are shown in FIG. 9, bar 1. HIV-1 Tat-mediated transactivation of the LTR led to [symbol] -galactosidase production, which was quantified 36 hours post-infection. Cells treated with ds siRNA targeting GFP-Nef (FIG. 9, bar 8) and targeting the mRNA encoding Tat sequence (FIG. 9, bar 9) served as positive controls. These controls previously showed decreased levels of β-galactosidase activity and viral infectivity (Jacque et al. 2002 Nature 418:435-8).

As shown in FIG. 9, ds siRNA directed against hCycT1 or CDK9 inhibited viral infectivity. Doubling dilutions of the inoculums are consistent with an 8-fold decrease in viral infectivity. Control experiments using siRNA duplexes containing mismatched sequences (see Experimental Procedures) and an unrelated ds siRNA against the RFP sequence showed no antiviral activities. Consistent with our previous results (Jacque et al., 2002, supra), siRNA targeting GFP-Nef and Tat led to an 8-fold decrease in viral infectivity. No significant toxicity or cell death was observed during these experiments, suggesting further that P-TEFb knockdown was not lethal. These results demonstrate that HIV infectivity can be modulated by siRNAs targeting CycT1 or CDK9, both components of P-TEFb, indicating that the use of siRNA targeting either subunit is a viable treatment for patients with HIV.

### Example 7

### P-TEFb Kinase Activity Remains Unchanged in P-TEFb Knockdown Cells

The observation that knockdown of P-TEFb did not lead to lethality was a surprising result as P-TEFb has an important role in transcription elongation and P-TEFb knockdown was previously shown to be required for early embryonic development in C. elegans (Shim et al. 2002 Genes Dev. 16:2135-2146). There were a number of possible explanations for these findings. One possibility was that mRNA and protein levels were significantly decreased in human cells transfected with siRNAs, but the low levels of P-TEFb that remained during RNAi may have been sufficient for sustaining cell viability and growth. Clarifying this further, cells may have been compensating for lower P-TEFb levels by converting inactive forms of the kinase to an activated form. This would suggest that a shift in equilibrium between inactive and active forms of P-TEFb may have occurred during hCycT1 or CDK9 knockdown that ultimately allowed cells to survive with lower levels of P-TEFb in the cells.

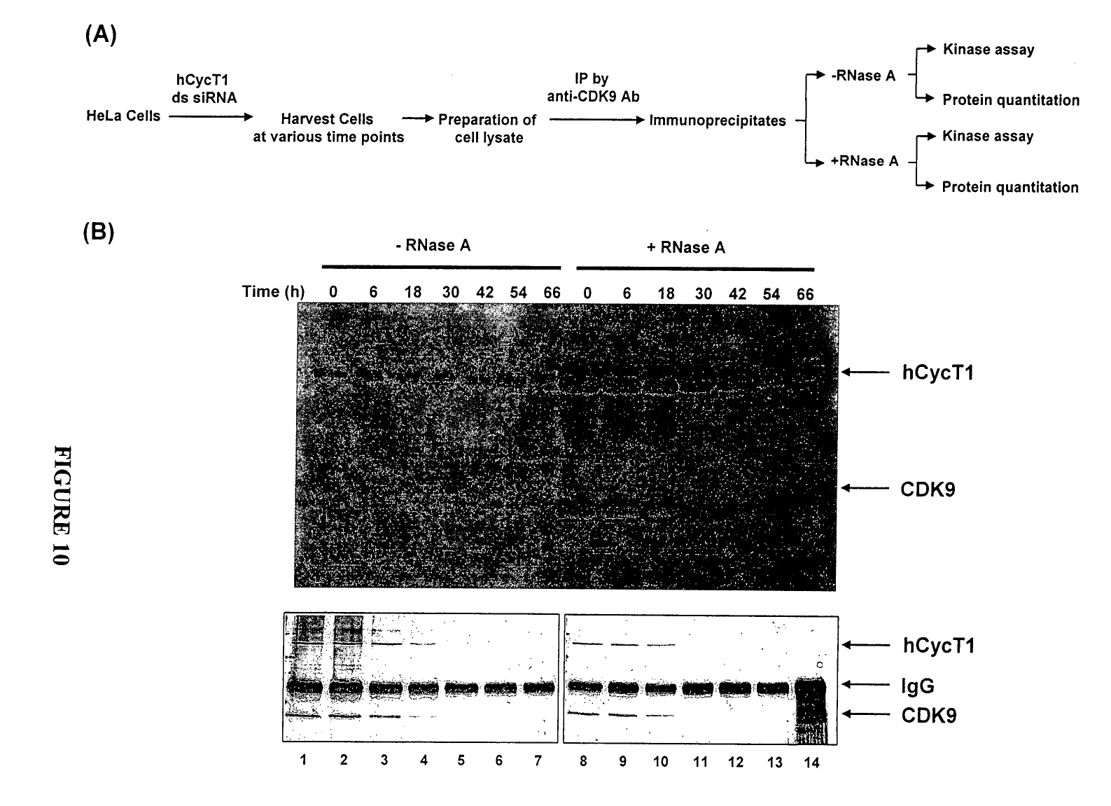

Recent studies have shown that P-TEFb is inhibited when in a complex with 7SK RNA and this 7SK-P-TEFb complex is the predominant form of P-TEFb present in the cell (Nguyen et al. 2001 Nature 414:322-5; Yang et al. 2001 Nature 414:317-22). If cells compensated for decreased P-TEFb levels by converting inactive 7SK-P-TEFb complexes to active P-TEFb complexes, then P-TEFb kinase activity in P-TEFb knockdown cells should be equal to the kinase activity in cells not exhibiting P-TEFb knockdown. To determine if levels of P-TEFb kinase activity were knocked down concomitantly with protein levels during RNAi, P-TEFb kinase activity was evaluated over the course of an hCycT 1 knockdown time course experiment (see experimental design in FIG. 10A).

Immunoprecipitation was carried out with anti-CDK9 antibodies to isolate P-TEFb complexes to ensure specificity of P-TEFb associated kinase activity in our assays. Briefly, P-TEFb and its associated factors were affinity purified by anti-CDK9 immunoprecipitation from HeLa cell extract at various time points after hCycT1 siRNA transfection. Immunoprecipitates were then treated with (FIG. 10B; lanes 1-7 upper panel) or without (FIG. 10B; lanes 8-14 upper panel) RNase A. Both the RNase A treated and non-treated immunoprecipitates were split into three equal pools to evaluate the kinase activity and protein levels of P-TEFb isolated by immunoprecipitation. Kinase assays were performed on the anti-CDK9 immunoprecipitates at 37° C. for 1 h. hCycT1 and CDK9 proteins in the immunoprecipitates were eluted with SDS and resolved by 10% SDS-PAGE to evaluate P-TEFb autophosphorylation. Specificity of the protein bands was confirmed by immunoblotting with anti-hCycT1 or anti-CDK9 antibodies (data not shown). To evaluate P-TEFb knockdown and protein levels of the immunoprecipitates used in the kinase assay, hCycT1, Cdk9 and anti-CDK9 IgG were visualized by silver stain. As shown in FIG. 10B (bottom panels), the silver stain showed that both hCycT1 and Cdk9 levels had been knocked down during the time course experiment (time =30-66 h) and that the same amount of immunoprecipitate was loaded for each time point (IgG band).

P-TEFb kinase activity of cells transfected with hCycT1 siRNA and showing hCycT1 and Cdk9 knockdown between 30-66 h was compared to P-TEFb kinase activity at the time of transfection (time =0 h). Time 0 h was prior to any observable P-TEFb knockdown and should be representative of normal levels of P-TEFb kinase activity. The observed kinase activity of immunoprecipates not treated with RNase A was quantitatively the same at all time points despite the reduction in hCycT1 protein levels observed over time (FIG. 10B, lanes 1-7). These results indicated that although P-TEFb protein levels were knocked down in cells transfected with hCycT1 or CDK9 siRNAs, P-TEFb kinase activity was not significantly affected. These observations would therefore support the hypothesis that cells were able to survive P-TEFb knockdown because the level of P-TEFb kinase activity normally present in cells was retained even after hCycT1 or CDK9 knockdown.

The kinase activity of immunoprecipitates treated with RNase A prior to performing the kinase assay was also compared at time 0 h and various time points post-siRNA transfection. Immunoprecipitates from time 0 h treated with RNase A showed a significant increase in kinase activity as compared to immunoprecipitates from time 0 h not treated with RNase A (FIG. 10B; compares lanes 1 and 8). This increase in activity indicated that 7SK RNA had been degraded by RNase A, relieving inhibition of P-TEFb kinase activity, as observed previously (Nguyen et al. 2001 Nature 414:322-5; Yang et al. 2001 Nature 414:317-22). At later time points, the increase in P-TEFb kinase activity began to decrease as P-TEFb protein levels decreased. However, through to the last time points of the experiment where the highest levels of P-TEFb knockdown were sustained, P-TEFb kinase activity never decreased to levels below the kinase activity observed with immunoprecipates not treated with RNaseA (FIG. 10B; compare lanes 5-7 and 12-14). This result suggested that there was a critical threshold of kinase activity consistently maintained during P-TEFb knockdown in the presence or absence of 7SK. These results also indicated that in the presence of 7SK, a shift in the equilibrium between pools of kinase inactive 7SK-P-TEFb and kinase active P-TEFb complexes occurred to achieve the critical threshold required for viability. Altogether, these results strongly support the hypothesis that the lower level of P-TEFb protein expressed during RNAi was sufficient for sustaining a critical threshold of P-TEFb kinase activity required for carrying out essential cellular functions.

### Discussion of Examples 1-7

The results presented here provided new mechanistic data about the regulation of P-TEFb kinase activity in response to changes in protein expression using RNA interference. First, this analysis demonstrated that P-TEFb mRNA and protein levels could be knocked down with siRNAs targeted to either CDK9 or hCycT1. Another important discovery was that CDK9 protein levels depended on hCycT1 protein levels, indicating that the formation of the P-TEFb complex was essential for stabilizing CDK9 in the cells. Interestingly, CDK9 levels, although showing a striking initial decline, seemed to stabilize instead of steadily declining further over time along with Cyclin T1 (FIG. 2B, lanes 8-14), suggesting that a separate pool of CDK9 was bound to other proteins that could stabilize the kinase subunit. CDK9 has been shown to form chaperone complexes with heat shock proteins Hsp70 and kinase-specific chaperone Hsp90/Cdc37 (O'Keeffe et al. 2000 J Biol Chem. 275:279-287), raising the possibility that the pool of CDK9 still present during hCycT1 knockdown may have been stabilized by these chaperone interactions.

The most surprising finding of this study was that cells survived knockdown of P-TEFb by maintaining normal kinase activity in the wake of reduced protein expression. The cell viability associated with RNAi of P-TEFb could have suggested that P-TEFb function was not essential for cell viability, a conclusion that was contradictory to its central role in transcription elongation and its essential roles in transcription during C. elegans embryonic development (Shim et al. 2002 Genes Dev. 16:2135-2146). At later stages in the life cycle, however, it was still not entirely clear whether complete knockout of P-TEFb was lethal to cells. Insight into whether P-TEFb has some essential functions in adult cells was revealed by the studies herein. With P-TEFb kinase activity remaining virtually the same under both normal and P-TEFb knockdown conditions, significantly reducing P-TEFb protein levels did not appear to limit its ability to carry out essential functions. However, our kinase assays indicated that there was a critical threshold of P-TEFb kinase activity required to sustain cell viability.

The mechanism by which cells appeared to compensate for lower P-TEFb protein levels via converting kinase inactive P-TEFb to active P-TEFb suggested that at the very least P-TEFb kinase activity was essential for cell viability. With such a compensatory mechanism in place, however, cells must first have a system for monitoring P-TEFb proteins levels, and secondly, have a mechanism established for signaling the conversion of inactive P-TEFb to kinase active P-TEFb. Recent studies uncovering an inhibitory role for 7SK RNA in regulating P-TEFb activity (Nguyen et al. 2001 Nature 414:322-5; Yang et al. 2001 Nature 414:317-22) may in part explain how the switch between inactive and active forms was signaled. Under normal conditions, 7SK interacts with the majority of P-TEFb available in the cell rendering it inactive, an assertion supported by biochemical data documenting the strong interactions between 7SK and P-TEFb and its negative effect on P-TEFb kinase activity (Nguyen et al. 2001 Nature 414:322-5; Sano et al. 2002 Nat Med 8:1310-7; Yang et al. 2001 Nature 414:317-22). A shift in this standard equilibrium between inactive P-TEFb-7SK complexes and active P-TEFb may then be triggered in several ways as depicted in FIG. 11 and described below.

One possible trigger may result from exposure to stress like UV irradiation or transcriptional inhibitor actinomycin D, which induce the release of 7SK from P-TEFb and increase the levels of kinase active P-TEFb in the cell (Nguyen et al. 2001 Nature 414:322-5; Yang et al. 2001 Nature 414:317-22). Cardiac hypertrophy signaling pathways have also been shown recently to cause the release of P-TEFb from the 7SK inhibitor (Sano et al. 2002 Nat Med 8:1310-7). Similarly, HIV may elicit a shift in the equilibrium between P-TEFb and its interactions with 7SK to increase Tat transactivation. Our results showing that HIV replication is reduced despite normal levels of P-TEFb kinase activity suggested that Tat transactivation associated with normal rates of HIV replication required a larger concentration of activated P-TEFb than normally available in the cell. Therefore, HIV has likely developed a signaling mechanism for shifting the equilibrium between 7SK and P-TEFb. Although it is clear that there must be a signaling mechanism involved in regulating 7SK-P-TEFb interactions, what the signaling components are and how they associate with the 7SK-PTEFb complexes to trigger release of P-TEFb remains unknown.

Although knockdown of P-TEFb was able to support cell viability and showed no significant growth defects, P-TEFb knockdown did have an effect on gene expression. Most significantly, RNAi of P-TEFb resulted in a decrease of both Tat transactivation and HIV replication, suggesting that the amount of P-TEFb in cells was important for the fidelity of Tat transactivation and HIV replication. Previous studies including P-TEFb immunodepletion analyses, and studies with small molecule inhibitors of P-TEFb or anti-hCycT1 intrabodies also supported an important role for P-TEFb in Tat transactivation and HIV replication ( Bai et al. 2003 J. Biol. Chem. 278:1433-42; Chao et al. 2000 J. Biol. Chem. 275:28345-28348; Flores et al. 1999 Proc. Natl. Acad. Sci. USA 96:7208-13; Ghose et al. 2001 J. Virol. 75:11336-43; Macebo et al. 1997 Genes Dev 11:2633-2644). However, none of these studies specifically showed the effects of altering P-TEFb protein levels in vivo. The studies herein are the first to show that targeting P-TEFb by RNAi was not lethal and also decreased HIV transcription and viral replication. These results suggest that using siRNAs to target P-TEFb was a feasible new approach for developing therapeutic agents for AIDS.

In addition to the P-TEFb RNAi effects on HIV replication, changes in global gene expression observed in microarray analyses demonstrated that knockdown of P-TEFb also had an effect on expression of a variety of other genes (Y. L. Chiu and T. M. Rana, unpublished results). The microarray analysis showed that P-TEFb was required for regulating the expression of a broad range of genes involved in a host of different cellular processes, including cell division, development, and stress response. These results provided additional evidence that P-TEFb knockdown occurred and indicating that cellular concentration of activated P-TEFb regulated expression of a broad range of genes. These data also suggested that only a small fraction of P-TEFb regulatory functions have truly been defined and more directed studies of the genes and related cell processes controlled by P-TEFb will be performed in the future.

The results presented here raise new and intriguing questions about P-TEFb function and how it is regulated in the cell. This analysis suggests that there is a critical threshold for P-TEFb kinase activity required for cell viability and Tat transactivation and that there are built-in intracellular mechanisms that allow cells to cope with changes in P-TEFb protein levels. These results also demonstrated that knockdown of P-TEFb by RNAi is a novel tool that can be used to understand P-TEFb cellular functions and the functions of genes regulated by P-TEFb.

### Example 8

### Genome-Wide Analysis of Gene Expression in P-TEFb Knockdown HeLa Cells

To investigate the effects of P-TEFb knockdown on global gene expression, total mRNA was isolated from HeLa cells treated with and without siRNA directed against hCycT1 or CDK9 and the expression of various genes was analyzed using the GeneChip® Human Genome U133 (HG-U133) from Affymetrix. The HG-U133 includes HG-133A and HG-133B sets of arrays containing 22,283 and 22,645 genes, respectively. Another 100 human maintenance genes on both arrays serve as a tool to normalize and scale the data prior to making comparisons.

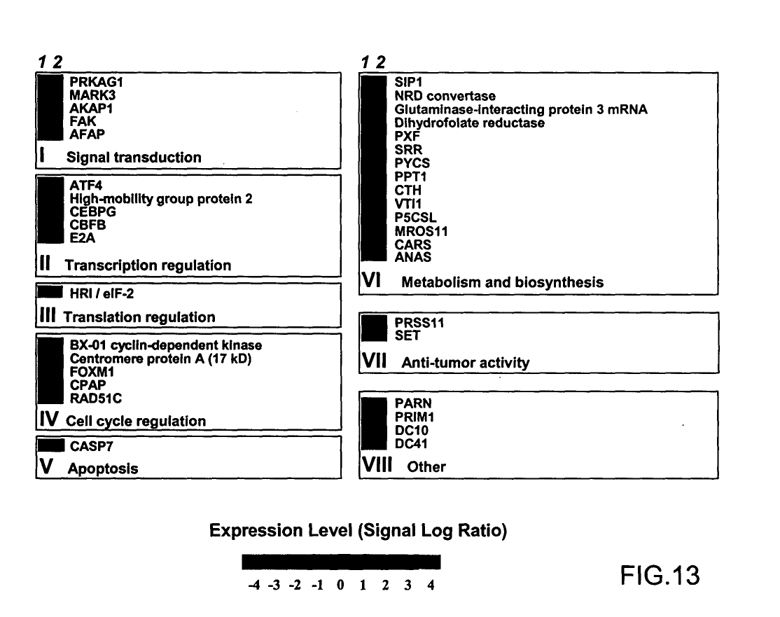

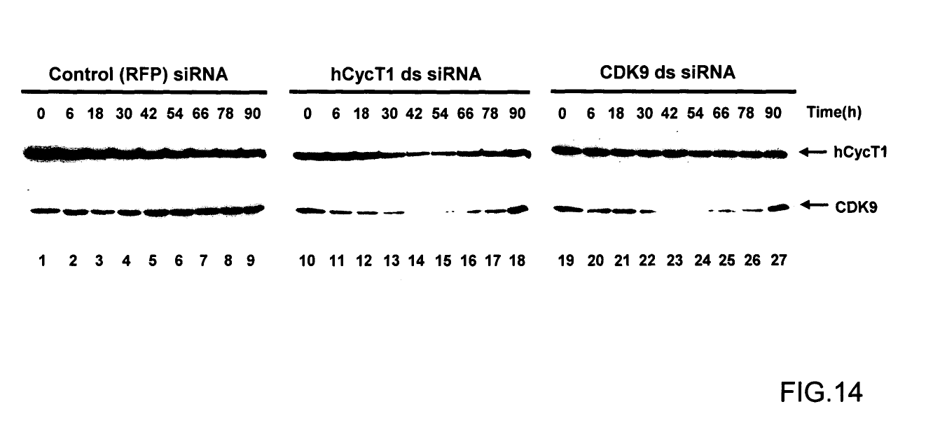

Briefly, HeLa cells were treated with and without ds siRNA directed against hCycT1 or CDK9, and total mRNA was isolated. Total mRNA was used to synthesize ds cDNAs, from which biotin-labeled cRNA was synthesized and fragmented. Fragmented cRNA was then subjected to high-density oligonucleotide microarray hybridization (GeneChip®) using Human Genome U133 from Affymetrix (see Experimental Procedures). Of the 44,928 genes expressed, 90 are displayed in FIGS. 12 and 13 by class, based on their putative functions. Each row represents one gene. Column 1 of FIGS. 12 and 13 indicates hCycT1 ds siRNA treatment and column 2 indicates CDK9 ds siRNA treatment. Both down—(FIG. 12) and up—(FIG. 13) regulated genes are shown. The brightness of each color reflects the magnitude of the gene expression level (Signal Log Ratio). See Experimental Procedures for details of analysis.

Genome-wide analysis of the gene expression profile of P-TEFb knockdown cells revealed that 201 genes out of 44,928 were down-regulated. A complete list of down- and up-regulated genes with names and accession numbers can be found in the Table below. Of those 201 genes, 53 are known and were classified according to their function or protein product activity (FIGS. 12 and 13). It is widely accepted that P-TEFb is a positive transcription factor during RNA pol II elongation. Down-regulated genes in the P-TEFb knockdown background are presumably those required by P-TEFb for normal levels of expression, especially at the transcription elongation level. These results indicate that P-TEFb regulation of various cellular genes is affected by siRNA targeting either CDK9 or CycT1.

Table 1 below summarizes the results of genome-wide analysis of gene expression in P-TEFb knockdown HeLa cells. Of 44,928 genes expressed, 390 are displayed. Each row represents one gene. The genes are listed by Accession number, and the magnitude of decrease (D) indicating downregulation, or increase (I) indicating upregulation, in Signal Log Ratio (SLR), is indicated.

| | CyclinT | | CDK9 | | | CyclinT | | CDK9 | |
|---|---|---|---|---|---|---|---|---|---|
| Accession | SLR | Change | SLR | Change | Accession | SLR | Change | SLR | Change |
| 230465_at | −4.5 | D | −1.02 | D | 225366_at | −1.29 | D | −1.74 | D |
| 234986_at | −2.96 | D | −1.57 | D | 226817_at | −1.27 | D | −1.11 | D |
| 229256_at | −2.88 | D | −2.52 | D | 230788_at | −1.26 | D | −1.3 | D |
| 225710_at | −2.75 | D | −2.68 | D | 226310_at | −1.26 | D | −1.16 | D |
| 226625_at | −2.5 | D | −1.17 | D | 224800_at | −1.26 | D | −1.79 | D |
| 224893_at | −2.31 | D | −1.33 | D | 238587_at | −1.24 | D | −2.66 | D |
| 222692_s_at | −2.19 | D | −1.85 | D | 226051_at | −1.24 | D | −0.96 | D |
| 225847_at | −2.16 | D | −1.89 | D | 225405_at | −1.24 | D | −1.03 | MD |
| 238417_at | −2.12 | D | −1.79 | D | 239269_at | −1.23 | D | −0.97 | D |
| 225479_at | −2.05 | D | −1.45 | D | 227740_at | −1.23 | D | −1.14 | D |
| 225032_at | −2.04 | D | −1.7 | D | 225575_at | −1.23 | D | −1.39 | D |
| 225179_at | −2.01 | D | −1.29 | D | 227853_at | −1.22 | D | −1.05 | D |
| 224989_at | −1.97 | D | −3.09 | D | 224617_at | −1.22 | D | −1.47 | D |
| 228745_at | −1.92 | D | −1.14 | D | 223441_at | −1.21 | D | −1.68 | D |
| 229553_at | −1.89 | D | −1.07 | D | 235258_at | −1.2 | D | −1.03 | D |
| 238320_at | −1.83 | D | −1.23 | D | 235125_x_at | −1.2 | D | −1.06 | D |
| 226685_at | −1.8 | D | −1.41 | D | 228531_at | −1.2 | D | −1.18 | D |
| 236262_at | −1.79 | D | −1.38 | D | 227685_at | −1.17 | D | −1.48 | D |
| 226785_at | −1.79 | D | −1.65 | D | 228611_s_at | −1.15 | D | −1.25 | D |
| 225319_s_at | −1.76 | D | −0.98 | D | 225571_at | −1.14 | D | −1.22 | D |
| 232125_at | −1.74 | D | −1.92 | D | 223006_s_at | −1.13 | D | −1.02 | D |
| 225266_at | −1.73 | D | −1.82 | D | 226671_at | −1.11 | D | −1.02 | D |
| 227052_at | −1.71 | D | −2.72 | D | 225813_at | −1.11 | D | −1.15 | D |
| 226982_at | −1.71 | D | −1.77 | D | 229450_at | −1.09 | D | −1.24 | D |
| 227112_at | −1.64 | D | −1.56 | D | 227771_at | −1.08 | D | −1.53 | D |
| 225750_at | −1.59 | D | −1.28 | D | 226045_at | −1.07 | D | −1.34 | D |
| 225242_s_at | −1.59 | D | −3.1 | D | 228479_at | −1.06 | D | −1.06 | D |
| 241879_at | −1.58 | D | −1.24 | D | 226115_at | −1.06 | D | −1.23 | D |
| 226962_at | −1.55 | D | −1.37 | D | 222996_s_at | −1.06 | D | −1.06 | D |
| 232079_s_at | −1.52 | D | −2.59 | D | 231716_at | −1.05 | D | −0.95 | D |
| 231550_at | −1.51 | D | −1.15 | D | 225607_at | −1.05 | D | −1.03 | D |
| 227062_at | −1.49 | D | −1 | D | 225912_at | −1.04 | D | −1.26 | D |
| 224874_at | −1.47 | D | −0.98 | D | 243357_at | −1 | D | −1.14 | D |
| 224691_at | −1.45 | D | −1.17 | D | 225725_at | −1 | D | −1.8 | D |
| 228418_at | −1.44 | D | 1.1 | I | 224209_s_at | −1 | D | −1.19 | D |
| 224597_at | −1.44 | D | −1.02 | D | 229399_at | −0.99 | D | −2.19 | D |
| 235198_at | −1.43 | D | −2.61 | D | 225192_at | −0.99 | D | −1.59 | D |
| 228220_at | −1.42 | D | −1.6 | D | 242931_at | −0.98 | D | −1.41 | D |
| 239503_at | −1.41 | D | −1.42 | D | 226711_at | −0.97 | D | −1.26 | D |
| 224702_at | −1.4 | D | −1.59 | D | 229010_at | −0.96 | D | −1.17 | D |
| 228708_at | −1.39 | D | −1.71 | D | 227082_at | −0.96 | D | −1.56 | D |
| 223571_at | −1.38 | D | −1.02 | D | 226312_at | −0.96 | D | −0.95 | D |
| 243495_s_at | −1.35 | D | −1.72 | D | 226237_at | −0.96 | D | −2.07 | D |
| 229461_x_at | −1.34 | D | −2.16 | D | 225950_at | −0.96 | D | 1.38 | I |
| 227312_at | −1.34 | D | −1.12 | D | 229075_at | 0.95 | I | 1.07 | I |
| 225522_at | −1.34 | D | −1.22 | D | 231094_s_at | 0.97 | I | 0.99 | I |
| 226390_at | −1.33 | D | −1.07 | D | 223039_at | 0.97 | I | 1.15 | I |
| 226254_s_at | −1.33 | D | −1.83 | D | 228867_at | 1 | I | 1 | I |
| 224618_at | −1.33 | D | −1.43 | D | 225836_s_at | 1 | I | 1.01 | I |
| 226633_at | −1.32 | D | −1.55 | D | 224824_at | 1.02 | I | 1.26 | I |
| 241453_at | 1.04 | I | 1.51 | I | 64883_at | −1.11 | D | −0.97 | D |
| 223024_at | 1.04 | I | 1.11 | I | 35148_at | −2.21 | D | −2.71 | D |
| 226181_at | 1.05 | I | 1.18 | I | 32502_at | −1.38 | D | −0.96 | D |
| 223513_at | 1.06 | I | 1.06 | I | 221568_s_at | −1.3 | D | −1.07 | D |
| 238756_at | 1.07 | I | 1.18 | I | 221269_s_at | −1.06 | D | −1.12 | D |
| 228577_x_at | 1.07 | I | 1.34 | I | 219594_at | −1.26 | D | −1.35 | MD |
| 225456_at | 1.08 | I | 1.34 | I | 219297_at | −1 | D | 0.96 | I |
| 225549_at | 1.09 | I | 1.35 | I | 219023_at | −1.36 | D | 1.01 | I |
| 225125_at | 1.09 | I | 0.98 | I | 218706_s_at | −1.25 | D | −1.15 | D |
| 236641_at | 1.1 | I | 1.29 | I | 218618_s_at | −1.99 | D | −1.79 | D |
| 227356_at | 1.1 | I | 1.66 | I | 218196_at | −1.61 | D | −1.89 | D |
| 225837_at | 1.1 | I | 1.21 | I | 217887_s_at | −1.36 | D | −1.33 | D |
| 225060_at | 1.11 | I | 1.66 | I | 217765_at | −0.96 | D | −1.02 | D |
| 229603_at | 1.13 | I | 1.17 | I | 217738_at | −0.96 | D | −1.09 | D |
| 242617_at | 1.14 | I | 0.96 | I | 215489_x_at | −1.01 | D | −1.29 | D |
| 227249_at | 1.14 | I | 1.45 | I | 215177_s_at | −1.52 | D | −1.68 | D |
| 225834_at | 1.2 | I | 1.06 | I | 214975_s_at | −1.34 | D | −1.9 | D |
| 225195_at | 1.22 | I | 0.97 | I | 214895_s_at | −1.56 | D | −1.11 | D |
| 222431_at | 1.23 | I | 1.04 | I | 214443_at | −1.13 | D | −1.51 | D |
| 223307_at | 1.24 | I | 1.07 | I | 214121_x_at | −2.05 | D | −0.97 | D |
| 222425_s_at | 1.25 | I | 1.24 | I | 213805_at | −1.14 | D | −1.44 | D |
| 224626_at | 1.26 | I | 1.53 | I | 213623_at | −1.15 | D | −1.46 | D |
| 227157_at | 1.28 | I | 1.08 | I | 213552_at | −1.37 | D | −1.44 | D |
| 225649_s_at | 1.29 | I | 1.49 | I | 213508_at | −0.99 | D | −1.32 | D |
| 231855_at | 1.3 | I | 1.24 | I | 213372_at | −1.86 | D | −1 | D |
| 223700_at | 1.3 | I | 1.12 | I | 213151_s_at | −1.14 | D | −1.84 | D |
| 222416_at | 1.3 | I | 1.23 | I | 213012_at | −1.78 | D | −1.36 | D |
| 228812_at | 1.31 | I | 1.33 | I | 212993_at | −2.01 | D | −1.4 | D |
| 224601_at | 1.31 | I | 1 | I | 212919_at | −1.7 | D | −0.96 | D |
| 232235_at | 1.37 | I | 1.05 | I | 212731_at | −1.86 | D | −1.73 | D |
| 236300_at | 1.38 | I | 1.2 | I | 212690_at | −1.25 | D | −1.38 | D |
| 228507_at | 1.38 | I | 1.26 | I | 212589_at | −1.11 | D | 1.18 | I |
| 225006_x_at | 1.42 | I | 1.73 | I | 212489_at | −1.07 | D | −1.69 | D |
| 224560_at | 1.43 | I | 1.68 | I | 212488_at | −1.22 | D | −1.4 | D |
| 222889_at | 1.43 | I | 1.37 | I | 212340_at | −1.09 | D | −1.08 | D |
| 231579_s_at | 1.46 | I | 1.19 | I | 211985_s_at | −1.41 | D | −1.11 | D |
| 228597_at | 1.46 | I | 1.7 | I | 211708_s_at | −1.36 | D | −2.08 | D |
| 225865_x_at | 1.51 | I | 1.81 | I | 211162_x_at | −1.8 | D | −2.16 | D |
| 229700_at | 1.58 | I | 1.37 | I | 211161_s_at | −1.03 | D | −1.28 | D |
| 229299_at | 1.59 | I | 1.21 | I | 210797_s_at | −1.01 | D | −1.57 | D |
| 225343_at | 1.65 | I | 1.72 | I | 210749_x_at | −1.2 | D | −1.55 | D |
| 224819_at | 1.66 | I | 1.5 | I | 209877_at | −1.95 | D | −1.11 | D |
| 222844_s_at | 1.67 | I | 1.34 | I | 209748_at | −1.41 | D | 1.07 | I |
| 225261_x_at | 1.68 | I | 1.95 | I | 209468_at | −1.17 | D | −0.98 | D |
| 224715_at | 1.76 | I | 1.23 | I | 209406_at | −1.67 | D | −0.98 | D |
| 222843_at | 1.77 | I | 1.77 | I | 209380_s_at | −1.23 | D | −1.58 | D |
| 226546_at | 1.79 | I | 1.2 | I | 209064_x_at | −1.34 | D | −1.71 | D |
| 228992_at | 1.87 | I | 1.96 | I | 208867_s_at | −1.73 | D | −0.96 | D |
| 240983_s_at | 1.96 | I | 1.09 | I | 208796_s_at | −1.29 | D | −1.03 | D |
| 230251_at | 2.22 | I | 1.36 | I | 208779_x_at | −1.42 | D | −1.54 | D |
| 208712_at | −1.49 | D | −1.01 | D | 201409_s_at | −1.48 | D | 1 | I |
| 208051_s_at | −1.71 | D | −1.66 | D | 201389_at | −1.69 | D | −1.38 | D |
| 207357_s_at | −1.84 | D | −1 | D | 201324_at | −2.35 | D | −1.37 | D |
| 207169_x_at | −1.19 | D | −1.69 | D | 201149_s_at | −1.57 | D | −1.26 | D |
| 207147_at | −1.07 | D | −1.02 | D | 201015_s_at | −1.47 | D | −1.09 | D |
| 206584_at | −1.43 | D | −1.87 | D | 200831_s_at | −1.91 | D | −1.33 | D |
| 206382_s_at | −1.15 | D | −0.98 | D | 200600_at | −2.12 | D | −1.07 | D |
| 206315_at | −1.25 | D | −1.82 | D | 1007_s_at | −1.25 | D | −1.19 | D |
| 205905_s_at | −1.21 | D | −0.96 | D | 52164_at | 1.16 | I | 1.06 | I |
| 205808_at | −1.11 | D | −2.05 | D | 48808_at | 1.54 | I | 1.12 | I |
| 205619_s_at | −1.88 | D | −1.4 | D | 40189_at | 1.42 | I | 1.18 | I |
| 205594_at | −1.03 | D | −1.52 | D | 222103_at | 1.2 | I | 1.27 | I |
| 205552_s_at | −1.2 | D | −1.29 | D | 222000_at | 1.19 | I | 1.04 | I |
| 205383_s_at | −0.97 | D | −1.24 | D | 221931_s_at | 1.18 | I | 1.24 | I |
| 204855_at | −1.3 | D | −1.61 | D | 221802_s_at | 0.99 | I | 1.62 | I |
| 204794_at | −1.19 | D | −1.34 | D | 221739_at | 2.12 | I | 1.7 | I |
| 204731_at | −2.29 | D | −1.04 | D | 221521_s_at | 1.18 | I | 1.08 | I |
| 204540_at | −0.95 | D | −1.22 | D | 220607_x_at | 1.55 | I | 1.43 | I |
| 204396_s_at | −2.27 | D | −1.09 | D | 220178_at | 0.95 | I | 1.43 | I |
| 204379_s_at | −3.18 | D | −3.36 | D | 219502_at | 1.24 | I | 0.96 | I |
| 204364_s_at | −1.07 | D | −1.39 | D | 219306_at | 1.07 | I | 1.48 | I |
| 204298_s_at | −1.52 | D | −1.16 | D | 219204_s_at | 1.02 | I | 1.1 | I |
| 203743_s_at | −0.97 | D | −1.06 | D | 219162_s_at | 1.1 | I | 1.21 | I |
| 203679_at | −1.15 | D | −1.15 | D | 219148_at | 1.08 | I | 1.18 | I |
| 203650_at | −2.56 | D | −1.85 | D | 219045_at | 1.12 | I | 1.36 | I |
| 203646_at | −2.24 | D | −1.21 | D | 218979_at | 1.05 | I | 1.62 | I |
| 203325_s_at | −1 | D | −1.22 | D | 218945_at | 0.98 | I | 1.02 | I |
| 203231_s_at | −1.68 | D | −1.02 | D | 218886_at | 1.29 | I | 2.09 | I |
| 203203_s_at | −1.01 | D | 0.98 | I | 218883_s_at | 1.32 | I | 1.04 | I |
| 203153_at | −2.49 | D | −1.23 | D | 218875_s_at | 1.21 | I | 1.44 | I |
| 203071_at | −1.05 | D | −1.14 | D | 218755_at | 1.08 | I | 1.17 | I |
| 202893_at | −1.17 | D | −1.02 | D | 218602_s_at | 1.3 | I | 1.36 | I |
| 202686_s_at | −2.03 | D | −2.81 | D | 218585_s_at | 0.96 | I | 1.15 | I |
| 202667_s_at | −1 | D | −1.14 | D | 218563_at | 1.86 | I | 2.38 | I |
| 202502_at | −1.13 | D | 1.07 | I | 218545_at | 1.33 | I | 1.3 | I |
| 202351_at | −1.37 | D | −1.04 | D | 218542_at | 0.96 | I | 1.02 | I |
| 202342_s_at | −1.79 | D | −1.35 | D | 218460_at | 1.15 | I | 0.97 | I |
| 202067_s_at | −1.12 | D | −2.56 | D | 218458_at | 1.42 | I | 1.27 | I |
| 201971_s_at | −1.61 | D | −1.1 | D | 218373_at | 1.39 | I | 1.1 | I |
| 201943_s_at | −1.75 | D | −1.07 | D | 218294_s_at | 0.96 | I | 2.14 | I |
| 201942_s_at | −1.5 | D | −1.56 | D | 218131_s_at | 1.55 | I | 1.05 | I |
| 201941_at | −2.04 | D | −0.99 | D | 217959_s_at | 1.73 | I | 1.35 | I |
| 201940_at | −2.36 | D | −1.4 | D | 217958_at | 1.43 | I | 1.5 | I |
| 201852_x_at | −1 | D | −1.21 | D | 217794_at | 1.79 | I | 1.36 | I |
| 201681_s_at | −1.64 | D | −1 | D | 217791_s_at | 1.34 | I | 1.18 | I |
| 201617_x_at | −1.45 | D | −1.03 | D | 217736_s_at | 1.54 | I | 1.64 | I |
| 201602_s_at | −1.23 | D | −1.02 | D | 217127_at | 1.25 | I | 1.4 | I |
| 201506_at | −1.15 | D | −1.13 | D | 216484_x_at | 1.62 | I | 1.62 | I |
| 201490_s_at | −1.85 | D | −0.97 | D | 216483_s_at | 2.38 | I | 1.67 | I |
| 201469_s_at | −1.24 | D | −1.62 | D | 214949_at | 1.49 | I | 1.32 | I |
| 214948_s_at | 1.67 | I | 1.37 | I | 204616_at | 1.32 | I | 1.57 | I |
| 213959_s_at | 1.63 | I | 1.02 | I | 204228_at | 1.01 | I | 1.11 | I |
| 213761_at | 1.1 | I | 1.21 | I | 204203_at | 1.23 | I | 0.99 | I |
| 213682_at | 1.4 | I | 2.2 | I | 203905_at | 1.14 | I | 1.18 | I |
| 213523_at | 1.49 | I | 1.37 | I | 203790_s_at | 1.17 | I | 1.25 | I |
| 213359_at | 1.44 | I | 1.38 | I | 203745_at | 1.6 | I | 1.25 | I |
| 213322_at | 1.52 | I | 1.09 | I | 203740_at | 1.07 | I | 1.77 | I |
| 213310_at | 1.9 | I | 1.37 | I | 203563_at | 0.98 | I | 1.5 | I |
| 213047_x_at | 1.3 | I | 1.08 | I | 203494_s_at | 1.27 | I | 1.21 | I |
| 213007_at | 1.15 | I | 0.99 | I | 203341_at | 1.49 | I | 1.66 | I |
| 212943_at | 0.98 | I | 1.19 | I | 203324_s_at | 2.03 | I | 1.77 | I |
| 212815_at | 1.23 | I | 1.19 | I | 203150_at | 1 | I | 1.04 | I |
| 212688_at | 1.39 | I | 1.08 | I | 203126_at | 1.61 | I | 1.73 | I |
| 212651_at | 1.18 | I | 1.67 | I | 202580_x_at | 1.97 | I | 1.55 | I |
| 212483_at | 0.96 | I | 1 | I | 202569_s_at | 1.14 | I | 1.11 | I |
| 212474_at | 1.79 | I | 1.81 | I | 202536_at | 1.11 | I | 1.86 | I |
| 212444_at | 1.38 | I | 1.1 | I | 202534_x_at | 1.71 | I | 1.15 | I |
| 212367_at | 1.38 | I | 1.34 | I | 202533_s_at | 1.05 | I | 1.24 | I |
| 212297_at | 1.3 | I | 1.04 | I | 202402_s_at | 1.71 | I | 1.02 | I |
| 212021_s_at | 1.65 | I | 1.27 | I | 202399_s_at | 1.12 | I | 1.64 | I |
| 212020_s_at | 1.85 | I | 1.23 | MI | 202370_s_at | 2.07 | I | 1.88 | I |
| 211595_s_at | 1.12 | I | 1.28 | I | 202209_at | 2.34 | I | 2.48 | I |
| 211115_x_at | 1.1 | I | 1.37 | I | 201707_at | 1.27 | I | 1.29 | I |
| 211114_x_at | 1.01 | I | 1.25 | I | 201675_at | 1.04 | I | 1.43 | I |
| 210821_x_at | 1.41 | I | 1.26 | I | 201555_at | 1.14 | I | 1.08 | I |
| 209849_s_at | 0.96 | I | 0.98 | I | 201536_at | 1.02 | I | 1.04 | I |
| 209714_s_at | 1.27 | I | 1.23 | I | 201515_s_at | 1.3 | I | 1.46 | I |
| 209513_s_at | 1.69 | I | 1.3 | I | 201513_at | 1.19 | I | 1.31 | I |
| 209512_at | 1.85 | I | 1.12 | I | 201328_at | 1.35 | I | 1.25 | I |
| 209452_s_at | 3.58 | I | 3.31 | I | 201306_s_at | 1.2 | I | 1.28 | I |
| 209154_at | 0.97 | I | 1.28 | I | 201292_at | 1.01 | I | 1.3 | I |
| 209096_at | 2.29 | I | 2.29 | I | 201185_at | 1.1 | I | 1.07 | I |
| 208931_s_at | 1.13 | I | 1.48 | I | 201001_s_at | 1.5 | I | 1.49 | I |
| 208808_s_at | 1.27 | I | 1.52 | I | 200975_at | 1.13 | I | 1.03 | I |
| 208709_s_at | 1 | I | 1.39 | I | 200896_x_at | 1.48 | I | 1.45 | I |
| 208654_s_at | 1.38 | I | 1.58 | I | 200779_at | 2.27 | I | 1 | I |
| 208653_s_at | 1.05 | I | 1.29 | I | 200749_at | 2.56 | I | 2.52 | I |
| 208405_s_at | 1.65 | I | 1.81 | I | 200640_at | 1.34 | I | 1.38 | I |
| 208079_s_at | 1.27 | I | 1.47 | I | 200639_s_at | 1.18 | I | 1.35 | I |
| 207181_s_at | 1.22 | I | 1.17 | I | 200631_s_at | 1.3 | I | 1.15 | I |
| 207165_at | 1.16 | I | 1.01 | I | 200629_at | 1.33 | I | 0.97 | I |
| 206364_at | 1.03 | I | 1.02 | I | | | | | |
| 206085_s_at | 1.18 | I | 1.07 | I | | | | | |
| 205417_s_at | 1.69 | I | 1.31 | I | | | | | |
| 205063_at | 1.09 | I | 1.65 | I | | | | | |
| 205053_at | 1.5 | I | 1.11 | I | | | | | |
| 205047_s_at | 3.28 | I | 1.78 | I | | | | | |
| 204962_s_at | 1.4 | I | 2 | I | | | | | |
| 204634_at | 1.91 | I | 1.71 | I | | | | | |
| 204622_x_at | 1.16 | I | 1.01 | I | | | | | |

### Example 9

### P-TEFb is Required for Embryonic Transcription but is Relatively Non-Essential in Adults

Broadly speaking, about 50% (25 out of 53) of the known down-regulated genes (FIG. 12) are known or likely to be involved in controlling and mediating cell proliferation and differentiation. These genes can be further divided into three classes (FIG. 12, I-III). The first class (FIG. 12, I) includes genes directly linked to cellular proliferation and differentiation. Most of these genes belong to the protein tyrosine kinase (PTK) superfamily. PTKs catalyze phosphate transfer from ATP to tyrosine residues on protein substrates, activating numerous signaling pathways leading to cell proliferation, differentiation, migration, or metabolic changes and playing a prominent role in the control of a variety of cellular processes during embryonic development (Hubbard and Till (2000), Annual Review of Biochemistry, 69, 373-398). Two classes of PTKs are affected by P-TEFb knockdown: transmembrane receptor protein tyrosine kinases (RTKs) and non-receptor tyrosine kinases (NRTKs). AXL receptor tyrosine kinase (AXL), discoidin domain receptor 1 (DDR1), epidermal growth factor receptor (EGFR) and fibroblast growth factor receptor (FGFR) belong to the RTKs, while cell adhesion kinase (CAK) belongs to the NRTKs. AXL, DDR1 and EGFR are required for the epithelial-mesenchymal transition during embryonic development, which allows multicellular organisms to get past the blastula stage (Thiery (2002). Nature Reviews Cancer, 2, 442-454).

Transforming growth factor beta (TGF-beta), also down-regulated by P-TEFb knockdown (FIG. 12, I), binds to another membrane receptor family with diverse functions during embryonic development and adult tissue homeostasis(Attisano and Wrana (2002), Science, 296, 1646-1647; Massague (2000), Nature Reviews Molecular Cell Biology, 1, 169-178). The genes for pre-T/NK cell-associated proteins (fasciculation and elongation protein zeta 2) are preferentially expressed in early stages of human T/NK cells and brain, suggesting that they play a role in early development (Ishii et al. (1999), Proc. Natl. Acad. Sci. USA, 96, 3928-3933; Whitehouse et al. (2002), Eur. J. Biochem., 269, 538-545). Brain-derived neurotrophic factor (BDNF) has been implicated in activity-dependent plasticity of neuronal function and network arrangement (Yamada et al. (2002), Journal of Neuroscience, 22, 7580-7585).

A second class of genes affected by P-TEFb knockdown is functionally linked to the cell membrane and extracellular matrix (FIG. 12, II). Junction plakoglobulin (JUP) cooperates with beta-catenin and promotes epithelial growth and morphogenesis (Hatsell and Cowin (2001). Nat. Cell Biol., 3, E270-272). Moesin (MSN), a plasma membrane protein associated with the underlying cytoskeleton, determines cell shape and participates in adhesion, motility and signal transduction pathways (Bretscher et al. (2002). Nature Reviews Molecular Cell Biology, 3, 586-599). This class of genes is also required for embryonic development, especially for angiogenesis and neuronal development. For example, integrin functions in epithelial cell organization and synaptogenesis during development of the CNS (Hynes (2002), Nature Medicine, 8, 918-921; Milner and Campbell (2002), J. Neuroscience Research, 69, 286-291). Epithelial membrane protein 1 (EMP1), which is highly expressed by immature neurons in the embryonic stage, functions in neuronal differentiation and neurite outgrowth (Wulf and Suter (1999), Brain. Res. Dev. Brain Res., 116, 169-180).

A third class of genes is involved in signal transduction (FIG. 12, III), the events downstream of membrane receptor activation. The inositol 1,4,5-triphosphate receptor (ITPR1) responds to G-protein-coupled receptor activation(Katso et al. (2001). Annual Review of Cell & Developmental Biology, 17, 615-675), while mitogen-activated protein kinase 6 (MAPK6) propagates cell proliferation/differentiation signals from receptor tyrosine kinase activation (Hubbard and Till, 2000, supra; Katso et al., 2001, supra). Non-receptor type protein tyrosine phosphatase regulates phosphotyrosine signalling events during complex ectodermal-mesenchymal interactions that regulate mammalian limb development (Arregui et al. (2000), Neurochemical Research, 25, 95-105; Saxton et al. (2000), Nat. Genet., 24, 420-423). Protein phosphatase 1 (PPP1CB) regulates the phosphorylation status of anti-apoptotic and pro-apoptotic proteins and their cellular activity in the apoptosis cascade(Klumpp and Krieglstein (2002), Curr. Opin. Pharmacol., 2, 458-462). Dual specificity phosphatase 2 (DUSP2) participates in the regulation of intracellular signal transduction mediated by MAP kinases (Yi et al. (1995). Genomics, 28, 92-96).

There is cross talk among these three gene classes (FIG. 12, I, II, and III). For example, collagen (type III, alpha 1), an extracellular matrix membrane protein can be a direct ligand for tyrosine kinase receptors (class I) and integrin (class II) (Shrivastava et al. (1997), Mol. Cell., 1, 25-34). Coordinated regulation of PTK (class I) and protein phosphatase (class II) also acts at different steps in a common signal transduction process (Bauman and Scott (2002), Nature Cell Biology, 4, E203-206). Non-receptor type protein tyrosine phosphatase (class III) positively regulates BDNF-promoted (class I) survival of ventral mesencephalic dopaminergic neurons (Takai et al. (2002), J. Neurochem., 82, 353-364). FGFR (class I), EMP1 (class II), pre-T/NK cell-associated protein (fasciculation and elongation protein zeta 2; class I) and integrin (class II) all participate in neuronal development. Nedasin (S form, class X), which is predominantly expressed in the brain, plays a role in the formation and structural changes of synapses during neuronal development by modifying clustering of neurotransmitter receptors at synaptic sites (Kuwahara et al. (1999), J. Biol. Chem., 274, 32204-32214). Down-regulation of the genes coding for these last five proteins by P-TEFb knockdown indicates an important role for P-TEFb during neuronal development.

From the expression profile of these three classes of genes, it can be concluded that P-TEFb is essential for embryonic gene expression and development, while knockdown of its subunits (hCycT1 and CDK9) does not affect cellular viability at the adult stage. It has been proposed that the P-TEFb complex is required for global gene expression during embryonic development of C. elegans. (Shim et al. (2002), Genes Dev., 16, 2135-2146.) Knockdown of CDK9 or CycT1 siRNA in C. elegans embryos inhibits transcription of embryonic genes, including the MAP kinase pathway and cell cycle-related genes (Shim et al., 2002, supra). The non-essential nature of P-TEFb in adult tissues makes it an ideal therapeutic target for treatment of HIV/AIDS and disorders characterized by unwanted cellular proliferation using the methods of the present invention.

### Example 10

### Method of Treating Cancer by Inhibiting P-TEFb

An intriguing finding is that genes linked to embryonic development and showing down-regulation in P-TEFb knockdown cells (as described above) also participate in tumorogenesis and metastasis. Dysfunction of protein tyrosine kinases (FIG. 12, I) or aberrations in key components of the signaling pathways they activate can lead to severe pathologies such as cancer, diabetes and cardiovascular disease. For example, overexpression of EGFR (FIG. 12, I) has been implicated in mammary carcinomas, squamous carcinomas and glioblastomas (Schlessinger (2002), Cell, 110, 669). AXL, another receptor tyrosine kinase (FIG. 12, I), was originally identified with oncogenic potential and transforming activity in myeloid leukemia cells(Burchert et al. (1998), Oncogene, 16, 3177-3187). Elevated TGF-beta levels can contribute to tumor progression and metastasis (Attisano and Wrana, 2002, supra; Massague, 2000, supra). Lysyl oxidase (LOX class II), an extracellular matrix remodeling enzyme, is up-regulated in prostatic tumor, cutaneous and uveal cell lines (Kirschmann et al. (2002), Cancer Res., 62, 4478-4483). Down-regulating these genes by P-TEFb knockdown using siRNA targeting CDK9 or CycT1 thus provides a new therapeutic strategy for inhibiting tumorigenesis and metastasis.

Genes involved in mediating progression through the cell cycle and as checkpoints in cancer were regulated by P-TEFb (FIG. 12, IV). Cyclin G1 is the downstream target of the P53 pathway and plays a role in G2/M arrest, damage recovery and growth promotion after cellular stress (Kimura et al. (2001), Oncogene, 20, 3290-3300). Cyclin D, a cell-cycle regulatory protein essential for G1/S transition, has been identified as a potential transforming gene in lymphoma (Motokura and Arnold (1993), Curr. Opin. Genet. Dev., 3, 5-10). Misregulation of the activity of its partner, CDK4/6, by overexpression of Cyclin D leads to hyperproliferative defects and tumor progression (Ortega et al. (2002), Biochim. Biophys. Acta, 1602, 73-87). Several marker genes in cancer cells (class V) are also regulated by P-TEFb. For example, breast cancer-specific protein 1 (BCSG 1) is overexpressed in advanced, infiltrating breast cancer and colorectal tumors (Lu et al. (2001), Oncogene, 20, 5173-5185). Another example is soluble urokinase plasminogen activator receptor (SUPAR), which is present in high concentrations in cystic fluid form ovarian cancer, tumor tissue of primary breast cancer, and gynecological cancer (Riisbro et al. (2002), Clin. Cancer. Res., 8, 1132-1141; Wahlberg et al. (1998), Cancer Res., 58, 3294-3298). Although the functions of these marker genes are still unknown, their high correlation with cancer has been used for prognosis in cancer therapy. The down-regulation of cyclin D and cancer marker genes by P-TEFb knockdown offers a method of cancer therapy. Briefly, a therapeutically effective amount of one of more of the pharmaceutical compositions of the invention is administered to a patient having a disorder characterized by unwanted or aberrant cellular proliferation as described herein.

### Example 11

### Links Between P-TEFb and Stress Responses

Another interesting group of genes down-regulated by P-TEFb are those involved in responding to stress or oxidant-mediated regulation (FIG. 12, VII). There are three known major redox signaling systems in eukaryotic cells, namely glutathione/glutathione reductase, thioredoxin/thioredoxin reductase and glutaredoxin (GLRX, thioltransferase) (Powis et al. (1995), Pharmacology & Therapeutics, 68, 149-173). SH3 domain-binding glutamine-rich 3-like protein (SH3BGRL3) also belongs to the thioredoxin family. Glutathione S-transferase M4 (GSTM4) is involved in detoxifying reactive electrophiles, such as drug or foreign compounds, by catalyzing their reaction with glutathione (GSH) (Cotgreave and Gerdes (1998). Biochem. Biophys. Res. Commun. 242, 1-9). Oxidant-mediated regulation by GSH systems plays a direct role in cellular signaling through thiol-disulfide exchange reactions with membrane-bound receptor proteins, transcription factors, and regulatory proteins in the cell (Cotgreave and Gerdes, 1998, supra). During stress responses, redox regulation has an important function in biological events such as DNA synthesis, enzyme activation, gene expression and cell cycle regulation. Down-regulation of genes involved in these events by P-TEFb knockdown in cells indicates an important role for P-TEFb in stress responses, especially oxidant-mediated regulation of cell proliferation. Moreover, glutathione (GSH) has been linked to multi-drug resistance (Cotgreave and Gerdes, 1998, supra), promising evidence for P-TEFb as a therapeutic target in cancer therapy.

Unlike the covalent modifications in redox regulation described above, the Bcl-2-associated athanogene (BAG) family of modulating proteins (FIG. 12, VII) functions through alterations in conformation and influences signal transduction through non-covalent post-translational modifications (Takayama and Reed (2001), Nat. Cell Biol., 3, E237-241). The BAG family molecular chaperone regulator-2 (BAG-2) belongs to this family, which contains an evolutionarily conserved “BAG domain” that allows its members to interact with and regulate the Hsp 70 (heat shock protein 70) family of molecular chaperones (Takayama and Reed, 2001, supra). Like Hsp 70, BAG-family proteins have been reported to mediate the physiological stress signaling pathway that regulates cell division, death, migration and differentiation (Takayama and Reed, 2001, supra). P-TEFb has been shown to be recruited to heat shock loci in Drosophila melongaster and to co-localize with Hsp 70 and Hsp 90 upon heat shock stress (Lis et al. (2000), Genes Dev., 14, 792-803). Down-regulation of the BAG-2 gene in P-TEFb knockdown cells indicates an important role for P-TEFb in regulating Hsp70 molecular chaperones in human cells. CDK9 itself has been proposed to form complexes with Hsp 70 and Hsp90/cdc37, thereby involving this chaperone-dependent pathway in the stabilization/folding of CDK9 as well as the assembly of an active CDK9/CycT1 complex (O'Keeffe et al. (2000), J. Biol. Chem., 275, 279-287). Thus, given this evidence of P-TEFb involvement in the stress response, down-regulation of P-TEFb by siRNA targeting CyctI or CDK9 provides a method for the treatment of stress-related disorders, as well as aging and senescence. Briefly, a therapeutically effective amount of one of more of the pharmaceutical compositions of the invention is administered to an aged patient or a patient having a stress-related disorder, or disorder characterized by aberrant aging or senescence.

### Example 12

### Role of P-TEFb in Metabolism and Biosynthesis

Several genes involved in metabolism and biosynthesis are also regulated by P-TEFb (FIG. 12, IX). Human iron-sulfur protein or ferredoxin (FDX1) serves as an electron transport intermediary for mitochondrial cytochrome P450 involved in steroid, vitamin D, and bile acid metabolism(Lill et al. (1999), Biol. Chem., 380, 1157-1166). Low-density lipoprotein receptor-related protein 5 (LRP5) contains conserved modules characteristic of the low-density lipoprotein (LDL) receptor family, genetically associated with Type 1 diabetes(Figueroa et al. (2000), J. Histochem. Cytochem., 48, 1357-1368). Because alterations in LRP5 expression may be responsible for susceptibility to diabetes, LRP5 may therefore be a potential target for therapeutic intervention. The vacuolar (H+)-ATPases (or V-ATPases) function in the acidification of intracellular compartments in eukaryotic cells. Eukaryotic translation elongation factor 1, alpha 2 isoform (EEF 1A2), a key factor in protein synthesis, has been shown to have oncogenic properties: it enhances focus formation, allows anchorage-independent growth and decreases doubling time of fibroblasts (Anand et al. (2002), Nature Genetics, 31, 301-305). EEF1A2 is amplified in 25% of primary ovarian tumors, its expression makes NIH3T3 fibroblasts tumourigenic and it increases the growth rate of ovarian carcinoma cells (Anand et al., 2002, supra). Thus, downregulation of P-TEFb using siRNA targeting CDK9 or CycT1 is useful for treating disorders associated with unwanted cell proliferation, including cancer. Briefly, a therapeutically effective amount of one of more of the pharmaceutical compositions of the invention is administered to a patient having a disorder characterized by unwanted or aberrant cellular proliferation as described herein.

### Example 13

### Role of P-TEFb in Cell Cycle Regulation

Also affected by P-TEFb knockdown were genes involved in cell cycle regulation (FIG. 12, VI), which in turn controls proliferation and differentiation. The retinoblastoma (RB) protein regulates both the cell cycle and. tissue-specific transcription by modulating the activity of its associated factors (MacLellan et al. (2000), Mol. Cell Biol., 20, 8903-8915). Efforts to identify such cellular targets have led to the isolation of two novel proteins, RB-associated protein (RBP21) and RB- and p300-binding protein EID-1 (an E1A-like inhibitor of differentiation) (MacLellan et al., 2000, supra). Although its cellular function is still unclear, RBP21 is widely expressed in various human tissues and cancer cell lines. EID-1 is a potent inhibitor of differentiation, an activity that has been linked to its ability to inhibit p300 (and the highly related molecule, CREB-binding protein, or CBP) acetylation of histones (Miyake et al. (2000), Mol. Cell Biol., 20, 8889-8902). EID-1, which is rapidly degraded by the proteasome as cells exit the cell cycle, may act at a nodal point that couples exit from the cell cycle to transcriptional activation of genes required for differentiation (Miyake et al., 2000, supra). Regulation of EID-1 expression by P-TEFb knockdown provides evidence that P-TEFb is also involved in cell cycle regulation, especially RB-linked regulation of proliferation and differentiation. Ths is yet more evidence that downregulation of P-TEFb using siRNA targeting CDK9 or CycT1 is useful for treating disorders associated with unwanted cell proliferation, including cancer. In such a method, for instance, a therapeutically effective amount of one of more of the pharmaceutical compositions of the invention is administered to a patient having a disorder characterized by unwanted or aberrant cellular proliferation as described herein.

### Example 14

### Up-Regulated Genes

Four major classes of up-regulated genes were observed, including those involved in signal transduction (FIG. 13, I), transcription regulation (FIG. 13, II), cell cycle regulation (FIG. 11, IV) and metabolism and biosynthesis (FIG. 13, VI). This up-regulation may not be a direct effect of P-TEFb knockdown but rather a secondary or correlated effect, to which the cell responds by overexpressing certain genes to compensate the loss of function of genes modulated by P-TEFb. The up-regulation of genes involved in signal transduction, transcription, and cell cycle regulation (FIG. 13, I, II, and IV) suggests that these genes complement cellular functions in P-TEFb knockdown cells or play a role in overcoming effects of down-regulated genes. An interesting correlation was observed between up-regulation and the functions of two classes of genes, i.e., genes involved in transcription regulation and metabolism/biosynthesis (FIG. 13, III and VI). As one example, the translation regulator gene eIF-2, which controls a signaling pathway to activate genes involved in amino acid biosynthesis (Harding et al. (2000), Mol. Cell, 6, 1099-1108), was up-regulated.

### Example 15

### Expression of Breast Cancer-Specific Gene 1 (BSCG1) in T47D Breast Cancer Cells is Regulated by P-TEFb Silencing

Analysis of the GeneChip® data strongly suggests that P-TEFb regulates the expression of many genes involved in cancer. To test this hypothesis, the effects of P-TEFb knockdown in T47D cells, a breast cancer cell line, were measured. This cell line was isolated from ductal carcinoma and metastatic sites of human breast cancer tissue. T47D cells have tumorogenic activity and can form colonies in soft agar. Like other metastatic breast cancer cell lines, T47D cells over-express the breast cancer-specific gene, BCSG1 (Lu et al. (2002), J. Biol. Chem., 277, 31364-31372). BCSG1, also referred to as synuclein gamma or persyn, is not expressed in normal breast tissue or benign breast disease tissue, but is over-expressed in stage III/IV breast carcinomas (Ji et al. (1997) Cancer Research, 57, 759-64; Jia et al. (1999) Cancer Research, 59, 742-7). Over-expression of BCSG-1 in breast cancer cells leads to a significant increase in cell motility and invasiveness in vitro and progression of metastasis in vivo (Jia et al. (1999), supra). Recent studies suggest that the aberrant expression of BCSG1 in breast carcinomas is caused by transcriptional activation of the BCSG1 gene or mis-regulation at the transcriptional level (Jia et al. (1999), supra; Lu et al. 2002, supra).

As shown herein (see FIG. 12, V), BCSG1 is one of the cancer marker genes significantly down-regulated (˜8 fold) in P-TEFb-silenced cells. Since P-TEFb is a positive transcription factor, the down-regulation of BCSG 1 expression should occur at the transcription level, thus, P-TEFb silencing can be used to inhibit the growth of breast cancer cells.

Effect of specific silencing of P-TEFb on BCSG1 mRNA and Protein Levels in T47D Cells.

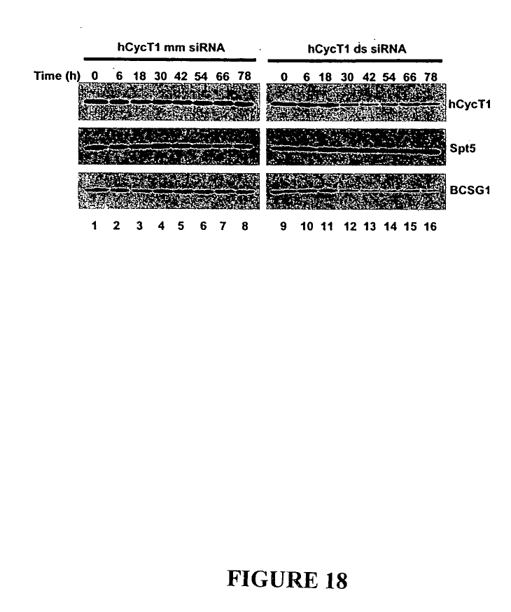

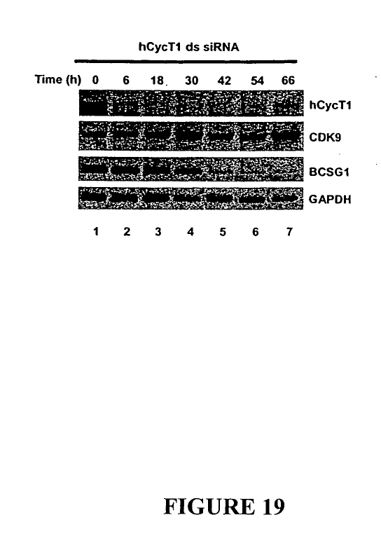

Specific silencing of hCycT1 in T47D cells can be achieved by duplex siRNA treatment; that down-regulation of hCycT1 levels by RNAi correlates with down-regulation of BCSG1 at the mRNA level (FIG. 19) and the protein level (FIG. 18).

T47D cells were transfected with hCycT1 ds siRNA (FIG. 19, lanes 1-7), harvested at various times after transfection, and mRNA was extracted. One-step RT-PCR was performed, setting the specific primer for hCycT1, CDK9, BCSG1 and GAPDH amplification (see Experimental Procedures). RT-PCR products were resolved in 1% agarose gel and viewed by ethidium bromide staining. The decrease in BCSG1 mRNA level at 42 hours after treatment with hCycT1 ds siRNA (FIG. 19, lane 5) shows that BCSG1 down-regulation occurs approximately 24 hours later than P-TEFb down-regulation.

Western blot analysis was used to demonstrate the effect of hCycT1 silencing on BCSG 1 expression in breast cancer cells. T47D cells were transfected with double-stranded (ds) siRNA hCycT1 (FIG. 18, lanes 9-16), or mutant siRNA (hCycT1 mismatch [mm; FIG. 18, lanes 1-8]) having 2 nucleotide mismatches between the target mRNA and the antisense strand of siRNA at the hypothetical cleavage site of the mRNA. Cells were harvested at various times post transfection, their protein content resolved on 10% SDS-PAGE, transferred onto PVDF membranes, and imrnunoblotted with antibodies against hCycT1 and BCSG1. For loading and specificity control, the same membranes were also blotted with antibodies against Spt5. This result confirms that P-TEFb knockdown down-regulates BCSG1 as described above. In addition, these results demonstrate that P-TEFb plays a role in transcribing the BCSG1 gene and that silencing P-TEFb by siRNA directed against its subunits can down-regulate BCSG1 expression by blocking its transcription.

### Example 16

### Expression of P-TEFb Silencing on Metastatic Potential and Growth of BSCG1-Expressing T47D Breast Cancer Cells

Effect of Specific Silencing of P-TEFb on T47D Cell Growth.

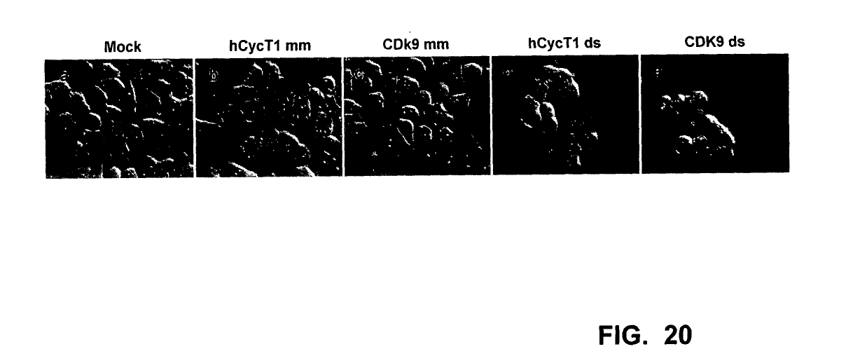

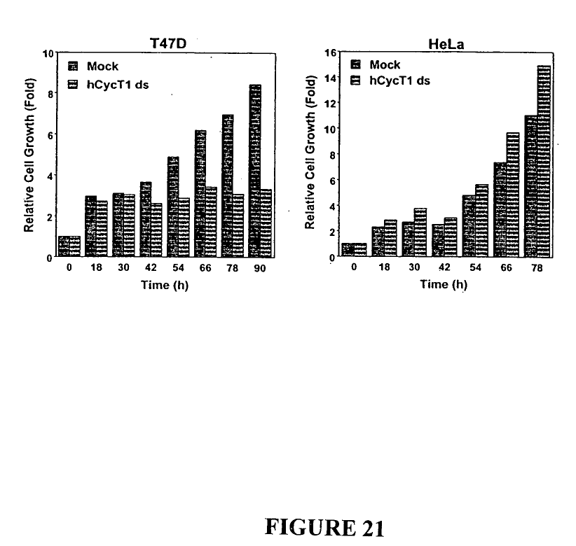

T47D cells were transfected with hCycT1 ds siRNA and harvested at various times after transfection. Total cell lysates were prepared, quantified as described herein and normalized to the amount of cell lysate at time 0; results are shown in FIG. 20. For comparison, the relative growth rate of HeLa cells treated with hCycT1 ds siRNA is also shown. Starting at 60% confluency, mock-treated T47D cells and those treated with mutant siRNAs reached ˜95% confluency at 54 hour post transfection (FIG. 20, panels a, b and c). T47D cells treated with siRNA directed against hCycT1 (FIG. 20, panel c) and CDK9 (FIG. 20, panel d) showed significant retardation in growth rate (˜70% confluency) and had smaller cell volume. The kinetics of T47D cellular growth (FIG. 21, left panel) shows that treatment with ds siRNA directed against hCycT1 prevents normal growth, while P-TEFb knockdown has no effect on the growth rate of HeLa cells (FIG. 21, right panel). Thus, P-TEFb silencing inhibits tumor cell growth.

The Effect of P-TEFb Silencing on T47D Tumor Cell Morphology.

The effects of P-TEFb silencing on tumor cell morphology were also examined in T47D cells. Control cells were mock-treated (FIG. 20, panel a), transfected with mutant siRNAs (hCycT1 mismatch [FIG. 20, panel b] or CDK9 mismatch [FIG. 20, panel c]) having 2 nucleotide mismatches between the target mRNA and the antisense strand of siRNA at the hypothetical cleavage site of the mRNA. Cells were also transfected with double-stranded (ds) siRNAs targeting hCycT1 (FIG. 20, panel d) or CDK9 (FIG. 20, panel e). FIG. 20 shows differential interference contrast images of living cells at 54 hours post transfection.

Effect of Specific Silencing of P-TEFb on Tumourigenic Activity of T47D Cells.

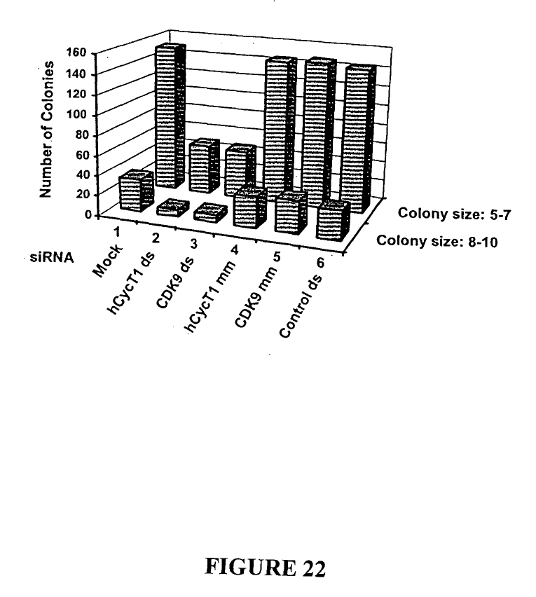

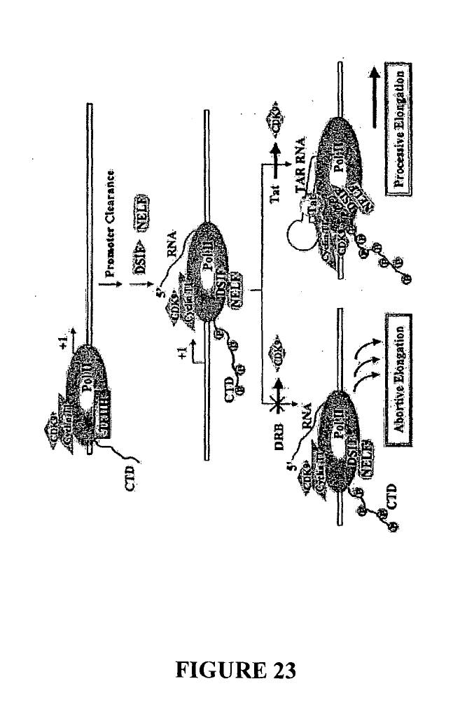

Tumourigenic activity of T47D cells was measured by assaying their anchorage-independent growth ability, as described above. The number of colonies containing 5-7 and 8-10 cells were counted separately at 99 hours post transfection. The results are the average of six randomly selected fields viewed at 200×. As shown in FIG. 22, P-TEFb silencing reduced the colony forming ability of T47D cells in soft agar. The number and size of colonies were reduced to 25% of controls (mock-treated or mutant siRNA-treated cells) after treating cells with ds siRNAs directed against the subunits of P-TEFb (FIG. 22). This result demonstrates that P-TEFb silencing reduces the tumourigenic activity of breast cancer cells.

Taken together, these results demonstrate silencing P-TEFb by siRNA directed against its subunits can affect both cell growth and tumorigenicity of cancer cells.

### Example 17

### Specific Silencing of P-TEFb in vivo

The effect of downregulating P-TEFb in vivo is assayed by administering siRNA targeted to CDK9 and/or CycT1 in an animal model. Any appropriate animal model can be used, for example, including but not limited to, rodent cancer models such as those available from the Mouse Models of Human Cancers Consortium (MMHCC) Repository (NCI, Frederick, Md.); the Oncomouse™ as described in U.S. Pat. Nos. 4,736,866, 5,087,571 and 5,925,803 (Taconic); or rodent or non-human primate models of HIV infection, such as the SCID-hu mouse.

For example, in a mouse model, the siRNA is administered using hydrodynamic transfection as previously described (McCaffrey, 2002, supra; Liu, 1999, supra), by intravenous injection into the tail vein (Zhang, 1999, supra); or by viral delivery (Xia, 2002, supra). At various time points after administration of the selected siRNA, mRNA levels for CDK9 and/or CycT1 can be measured. Additionally, the siRNA can be labeled, and the half-life of the siRNA molecules can be tracked using methods known in the art. Using electroporation, RNase III-prepared siRNA can be delivered into the post-implantation mouse embryos. 0.03 :g-0.3 :g siRNA can efficiently silence reporter gene expression in different regions of the neural tube or other cavities of the mouse embryo (Calegari (2002), supra). Using rapid injection of the siRNA-containing physiological solution into the tail vein of postnatal mice, 0.5-5 :g siRNA can cause 36±17%-88%±3% inhibition of target gene expression. The effect of RNAi is siRNA dose-dependent and can persist for approximately 4 days after siRNA delivery (Lewis (2002), supra). By direct injection, 5-40 :g siRNA can be used to silencing target gene expression in the liver, which is central to metabolism (Lewis (2002), supra; McCaffrey (2002), supra).

Any appropriate parameter can be observed to investigate the effect of P-TEFb expression. For example, changes in gene expression can be determined, such as changes in the expression of any one or more of the genes listed herein. In a mouse cancer model, appropriate parameters can include survival rates, tumor growth, metastasis, etc. In a simian HIV model, for instance parameters that can be determined include, but are not limited to, infectivity, viral load, survival rates, and rates and severity of secondary AIDS-associated illnesses.

Such models may also be useful for evaluating various gene delivery methods and constructs, to determine those that are the most effective, e.g., have the greatest effect, or have a desirable half-life or toxicity profile, for instance.

### Example 18

### Expression Patterns of P-TEFb

Expression of CDK9 in Embryonic and Adult Mouse Tissues

Bagella et al. (1998), supra, subjected paraffin-embedded mouse embryos from 12 to 17 days of gestation to in situ immunohistology and immunofluorescence by incubating with anti-CDK9/PITALRE antibody followed by peroxidase or FITC-conjucated secondary antibodies. This study indicated that murine CDK9/PITALRE protein appeared to be expressed predominantly in tissues that are terminally differentiated such as the developing brain and the dorsal root ganglia, areas of skeletal muscle, cardiac muscle, and the lining of the developing intestinal epithelium. Analysis of expression pattern of the murine CDK9/PITALRE protein in adult mouse tissues by immunoblotting indicates that murine CDK9/PITALRE expression is ubiquitous, however, steady-state protein levels are markedly higher in the brain, liver, lung, spleen and kidney. Kinase activity of CDK9/PITALRE kinase also detected in the same adult tissues and was highest in the mouse brain, liver, spleen and lung. Only minimal kinase activity was found in the heart, muscle, and kidney. This indicates that siRNA directed towards one or more subunits of P-TEFb may be particularly therapeutically effective in cancers of tissues in which CDK9 activity is high.

Expression of hCyc-T1 in Adult Human Tissues

De Luca et al. (2001), J. Histochem. Cytochem., 49(6), 685-92, investigated the expression pattern of CycT1 in adult human tissues by immunohistochemistry by incubating with polyclonal anti-CycT1 antibody. CycT1 expression is ubiquitous, with higher immunoreactivity in some tissues of mesenchymal origin, such as cardiovascular and connective tissues, skeletal muscle cells, myocardial cells, adipocytes, chondrocytes and endothelial cells, blood and lymphoid tissues. Astrocytes, oligodendroglial and microglial cells of the brain tissue also had a high level expression of Cyclin Ti while endocrine and reproductive systems showed low Cyclin T1 expression. Again, this is evidence that siRNA directed towards one or more subunits of P-TEFb may be particularly therapeutically effective in cancers of tissues in which CDK9 activity is high.

### Experimental Procedures for Examples 19-25

siRNA preparation

Design of siRNAs Targeting Spt5

The targeted region in the mRNA, and hence the sequence of Spt5-specific siRNA duplexes, was designed following the guidelines provided by Dharmacon (Lafayette, Colo.). Briefly, beginning 100 bases downstream of the start codon, the first AA dimer was located and then the next 19 nucleotides following the AA dimer were recorded. Ideally, the guanosine and cytidine content (G/C content) of the AA-N19 21 base-sequence would be less than 70% and greater than 30%. The search was continued downstream until the conditions were met. The 21-mer sequence was subjected to a BLAST search against the human genome/NCBI EST library to ensure only the desired gene was targeted. The siRNA sequence targeting hSpt5 was from position 407-427 relative to the start codon. siRNA sequences used in the experiments described herein were: hSpt5ds (5′-AACTGGGCGAGTATTACATGAdTdT-3′) (SEQ ID NO: 8); h Spt5 mm (5′-AACTGGGCGGATATTACATGAdTdT-3′) (SEQ ID NO: 9); Tat ds (5′-GAAACGUAGACAGCGCAGAdTdT-3′) (SEQ ID NO: 18); GFP ds (5′-GCAGCACGACUUCUUCAAGdTdT-3′) (SEQ ID NO: 19); and RFP ds (5′-GUGGGAGCGCGUGAUGAACdTdT-3′) (SEQ ID NO: 20). Underlined residues represent the sequences mismatched to their targets.

Using the guidelines provided by Dharmacon (Lafayette, Colo.) as discussed above, other potential siRNA sequences targeting Spt5, as well as siRNA sequences targeting Spt4 or Spt6, can be identified.

SiRNA Synthesis and Maintenance

21-nt RNAs were chemically synthesized as 2′ bis(acetoxyethoxy)-methyl ether-protected oligos by Dharmacon (Lafayette, Colo.). Synthetic oligonucleotides were deprotected, annealed to form dsRNAs and purified according to the manufacturer's recommendation. Successful duplex formation was confirmed by 20% non-denaturing polyacrylamide gel electrophoresis (PAGE). All siRNAs were stored in DEPC (0.1% diethyl pyrocarbonate)-treated water at −80° C.

Culture and Transfection of Cells

HeLa cells were maintained at 37° C. in Dulbecco's modified Eagle's medium (DMEM, Invitrogen) supplemented with 10% fetal bovine serum (FBS), 100 unit/ml penicillin and 100 μg/ml streptomycin (Invitrogen). Magi (multinucleate activation of galactosidase indicator) cells harboring the endogenous HIV LTR-β-gal gene were maintained at 37° C. in DMEM, supplemented with 10% FBS, 0.2 mg/ml Geneticin (G418), and 0.1 mg/ml hygromycin B (Roche Molecular Biochemicals). Cells were regularly passaged at sub-confluence and plated at 70% confluency 16 hours before transfection. Lipofectamine™ (Invitrogen)-mediated transient cotransfections of reporter plasmids and siRNAs were performed in duplicate 6-well plates (Falcon) as described for adherent cell lines by the manufacturer. A standard transfection mixture containing 100-150 nM siRNA and 9-10 μl Lipofectamine™ in 1 ml serum-reduced OPTI-MEM™ (Invitrogen) was added to each well. Cells were incubated in transfection mixture at 37C. for 6 hours and further cultured in antibiotic-free DMEM. For Western blot analysis at various time intervals, the transfected cells were harvested, washed twice with phosphate buffered saline (PBS, Invitrogen), flash frozen in liquid nitrogen, and stored at −80° C. for analysis. For in vivo assays of Tat-mediated transactivation at 48 hours post transfection, Magi cells were directly stained for β-galactosidase or flash frozen in liquid nitrogen and stored at −80° C. for β-galactosidase assays as described below.

Western Blotting

Cells treated with siRNA were harvested as described above and lysed in ice-cold reporter lysis buffer (Promega) containing protease inhibitor (complete, EDTA-free, 1 tablet/10 ml buffer, Roche Molecular Biochemicals). After clearing the resulting lysates by centrifugation, protein in clear lysates was quantified by Dc protein assay kit (Bio-Rad). Proteins in 30-60 μg of total cell lysate were resolved by 10% SDS-PAGE, transferred onto a polyvinylidene difluoride membrane (PVDF, Bio-Rad), and immuno-blotted with antibodies against hCycT1 (Santa Cruz Biotech) and Spt5 (Pharmingen). Protein content was visualized with a BM Chemiluminescence Blotting Kit (Roche Molecular Biochemicals). The blots were exposed to x-ray film (Kodak MR-1) for various times (1 second to 5 minutes).

RT-PCR for Amplification of mRNA

Total cellular mRNA is prepared from HeLa cells with or without siRNA treatment using a Qiagen RNA mini kit, followed by an Oligotex™ mRNA mini kit (Qiagen). RT-PCR is performed using a SuperScript™ One-Step RT-PCR kit with platinum Taq (Invitrogen) and 40 cycles of amplification. Each RT-PCR reaction included 100 ng total cellular mRNA, gene-specific primer sets for amplification of the target gene, e.g., hCycT1, Spt4, Spt5, or Spt6 (0.5 μM for each primer), 200 μM dNTP, 1.2 mM MgSO₄ and 1U of RT/platinum Taq mix. Primer sts for hSpt5 produced 2.6 Kb products while hCycT1 produced 1.8 Kb products. RT-PCR products were resolved in 1% agarose gel and viewed by ethidium bromide staining.

Plasmids Harboring the HIV-1 Tat Sequence

pTat-RFP plasmids were constructed by fusing the DNA sequence of HIV- 1 Tat with DNA sequences of DsRedl-N1, harboring coral (Discosoma spp.)-derived red fluorescent protein (RFP), per the manufacturer's recommendation (Clontech). A cytomegalovirus promoter was used to drive the expression of Tat-RFP fusion proteins, which were easily visualized in living cells by fluorescence microscopy (Zeiss). Expression of Tat-RFP fusion proteins can also be quantified by directly exciting the RPF fluorophore in clear cell lysates and measuring fluorescence, as described below.

β-Galactosidase Staining of Cells

Magi cells were transfected with Tat-containing plasmids in the absence or presence of siRNAs. At 48 hours post-transfection, cells were washed twice with PBS and fixed for 5 minutes in fixative (1% formaldehyde and 0.2% glutaraldehyde in PBS) at room temperature. After washing twice with PBS, cells were covered with staining solution (PBS containing 4 mM potassium ferrocyanide, 4mM potassium ferricyanide, 2 mM MgCl₂ and 0.4 mg/ml X-gal [Promega]) and incubated at 37° C. for exactly 50 minutes. Plates were then washed twice with PBS, and cell counts made of the number of β-galactosidase positive (blue) cells per 100-power field.

β-Galactosidase Enzyme Assay

Magi cells were transfected with Tat-containing plasmids in the absence or presence of siRNAs. At 48 hours post transfection, cells were harvested and clear cell lysates were prepared and quantified as described above. Total cell lysate (120 μg) in 1×reporter lysis buffer (150 μl) was subjected to standard β-galactosidase assay by adding 150 μl 2×β-galactosidase assay buffer (Promega) and incubating at 37° C. for 30 minutes. The reactions were stopped by adding 500 μl 1M sodium carbonate and briefly vortexing. Absorbance was read immediately at 420 nm. The amount of Tat-RFP protein was determined by fluorescence measurements on a PTI (Photon Technology International) fluorescence spectrophotometer. 300 μg of cell lysates was subjected to spectrophotometer with slit widths set at 4 nm for both excitation and emission wavelengths. All experiments were carried out at room temperature. Fluorescence of Tat-RFP in the cell lysate was detected by exciting at 568 nm and recording the emission spectrum from 588 nm to 650 nm; the spectrum peak at 583 nm represents the maximum fluorescence intensity of Tat-RFP. Tat transactivation was determined by calculating the ratio of β-galactosidase activity (absorbance at 420 run) of the pTat-RFP transfected cells to that of cells without pTat-RFP plasmid treatment. The inhibitory effect of siRNA treatment was determined by normalizing Tat-transactivation activity to the amount of Tat-RFP protein (represented by RFP fluorescence intensity) in the presence and absence of siRNA.

In vivo Fluorescence Analysis

pEGFP-C1 reporter plasmids (1 μg) and siRNA (100 nM) are cotransfected into HeLa cells by Lipofectamine™ as described above, except that cells are cultured on 35 mm plates with glass bottoms (MatTek Corporation, Ashland Mass.) instead of standard 6-well plates. Fluorescence in living cells is visualized 50 hours post transfection by conventional fluorescence microscopy (Zeiss). For GFP fluorescence detection, a FITC filter is used.

GeneChip® Experiments

Total cellular mRNA is prepared from HeLa cells with or without Spt5 ds siRNA treatment using a Qiagen RNA mini kit followed by Oligotex™ mRNA mini kit. Double-stranded cDNAs are synthesized from 2 μg total mRNA using the Superscript Choice System for cDNA synthesis (Invitrogen) with the T7-(dT)24 primer following the manufacturer's recommendations. cDNAs are cleaned up by phase lock gel (PLG) (Brinkman Instrument)phenol/chloroform extraction and concentrated by ethanol precipitation. Biotin-labeled cRNA is synthesized from cDNA by in vitro transcription using the Bioarray High Yield RNA transcript Labeling Kit (Affymetrix) following the vendor's recommendation. In vitro transcription products are cleaned up using RNeasy spin columns (Qiagen) and fragmented into 35-200 base units by metal-induced hydrolysis in fragmentation buffer (40 mM Tris-acetate, pH 8.1, 100 mM KOAc, 30 mM MgOAc). Fragmented cRNA is then subjected to Affymetrix Human Genome U133A and U133B GeneChip® sets in hybridization buffer (100 mM MES, 1M NaCl, 20 mM EDTA, 0.01% Tween-20). GeneChip® images are then analyzed with Affymetrix Microarray Suite V5.0 and Affymetrix Data Mining Tool V3.0.

Magi Infectivity Assay

HeLa-CD4-LTR/β-gal indicator (Magi) cells (Kimpton and Emerman, J. Virol. 66:2232-2239 (1992)) were plated in 24-well plates (7.5×10⁵ cells per well) and transfected with siRNAs as previously described (Jacque et al., Nature 418:435-438 (2002)). Briefly, siRNA (60 pmol) was transfected into cells using Oligofectamine™ (2 μl, Invitrogen) for 3 hours in serum-free DMEM (GIBCO). Cells were rinsed twice and top-layered in 500 μl of DMEM-10% FBS. Twenty-four hours after transfection, cells were trypsinized and seeded in 96-well microtiter plates (4×10⁴ cells per well), incubated 3 hours and infected with HIV_{NL-GFP}, an infectious molecular clone of HIV-1.. HIV-1 virions (normalized to RT (reverse transcriptase) activity in cpm) were added in doubling dilutions to duplicate wells. Forty-eight hours post-infection, the cells are harvested to quantify β-galactosidase activity and protein levels.

### Example 19

### Specific Silencing of hSpt5 Exression by siRNA in HeLa Cells

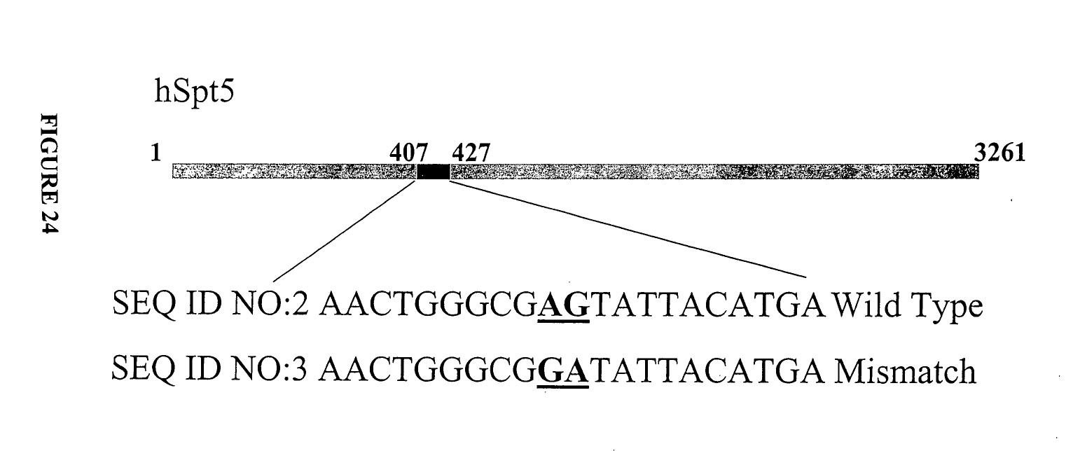

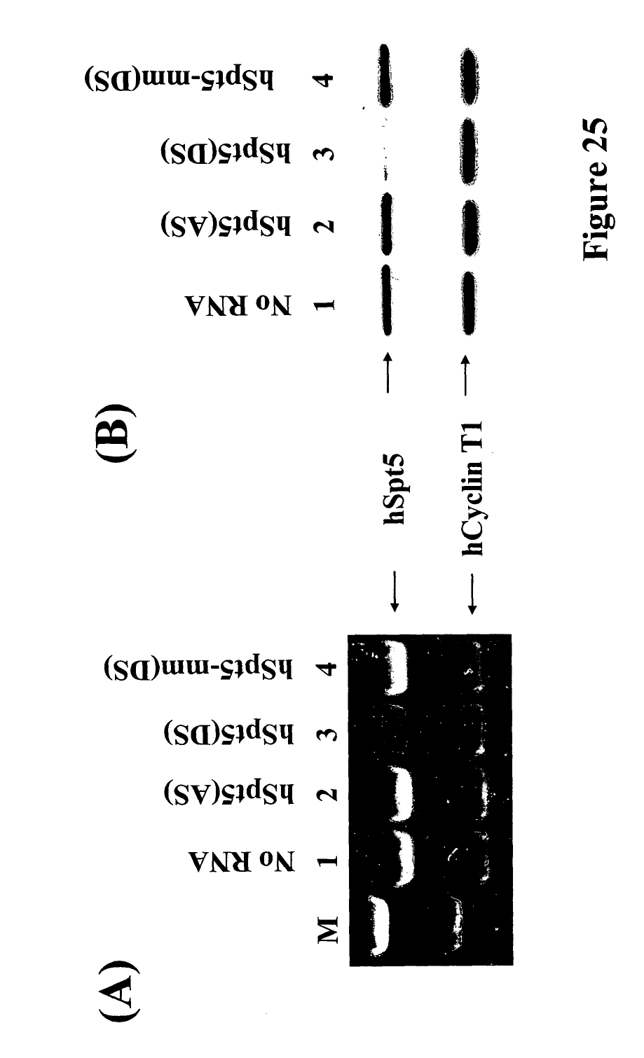

To inhibit hSpt5 expression in a cultured human cell line using RNAi, siRNA targeting an hSpt5 sequence from position 407 to 427 relative to the start codon was designed (FIG. 24). Magi cells were then transfected with hSpt5 duplex siRNA using Lipofectamine (Invitrogen). To evaluate the effects of hSpt5 RNAi, total cell lysates were prepared from siRNA-treated cells harvested at various time points after transfection. hSpt5 mRNA or protein levels were then analyzed by RT-PCR or western blot using anti-hSpt5 antibodies, respectively. These experiments showed that cells transfected hSpt5 siRNA had significantly lowered hSpt5 mRNA (FIG. 25A, lane 3) and protein expression (FIG. 25B, lane 3), indicating that RNAi of hSpt5 had occurred successfully. This knockdown effect was dependent on the presence of a 21 -nt siRNA duplex harboring a sequence complementary to the mRNA target. As shown in FIGS. 25A and 25B, mock-treated (no siRNA) (lane 1), single-stranded antisense hSpt5 siRNA (lane 2), mismatched hSpt5 duplex siRNA (lane 4), containing two nucleotide mismatches between the target mRNA and siRNA antisense strand at the putative cleavage site of the target mRNA (FIG. 24) did not affect hSpt5 mRNA or proteins levels. This suggested that hSpt5 knockdown was specific to duplex siRNA exactly complementary to the hSpt5 mRNA target. In evaluating either mRNA or protein levels, human Cyclin T1 (hCycT1) was used as an internal control, showing that the effects of hSpt5 siRNA were specific to hSpt5 and did not effect hCycT1 mRNA or protein levels (FIGS. 25A and 25B, lower panel). Taken together, these results suggested that hSpt5 knockdown was sequence specific and led to significantly decreased hSpt5 mRNA and proteins levels.

### Example 20

### Kinetics of P-TEFb mRNA Interference in HeLa Cells

RNA interference is a highly efficient process because a few dsRNA molecules are sufficient to inactivate a continuously transcribed target mRNA for long periods of time. Experiments have shown in plants and worms (Cogoni and Macino Nature 399:166-169 (1999); Dalmay et al., Cell 101:543-553 (2000); Grishok et al., Science 287:2494-2497 (2000)) that this inactivation can spread throughout the organism and is often heritable to the next generation. Mutations in genes encoding proteins related to RNA-dependent RNA polymerase (RdRP) affect RNAi-type processes in Neurospora, Caenorhabditis elegans, and plants (Cogoni and Macino (1999), supra; Dalmay et al. (2000), supra; Lipardi et al. Cell 107:297-307 (2001); Mourrain et al., Cell 101:533-542 (2000); Smardon et al., Curr. Biol. 10:169-178 (2000)), and the involvement of RdRP in amplifying RNAi has been postulated (Lipardi et al. (2001), supra).

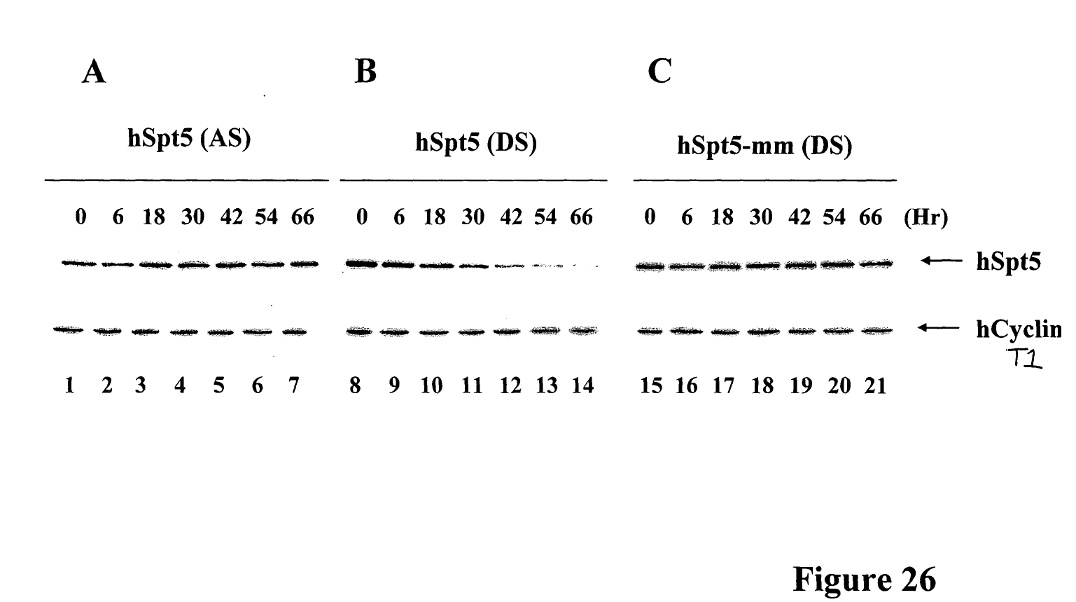

Having established that hSpt5 could be knocked down using RNAi, the kinetics of hSpt5 knockdown were examined. To perform kinetic experiments, hSpt5 duplex siRNA, single-stranded antisense hSpt5 siRNA, or mismatch duplex hSpt5 siRNA were transfected into Magi cells. Cell lysates were collected at various time points to assay for protein levels during hSpt5 knockdown. Briefly, cells were harvested at various times and protein contents were resolved on 10% SDS-PAGE, transferred onto PVDF membranes, and immunoblotted with antibodies against hCycT1 and Spt5. Immunoblot analysis revealed the timing of gene suppression and persistence of hSpt5 RNAi effects in Magi cells during the time course experiment (FIG. 26). hSpt5 knockdown was first observed between 6-18 h post-transfection, with maximum knockdown occurring at 42-66 h post transfection (FIG. 26B, lane 8-14). Protein levels gradually recovered to normal levels between 66-90 h (data not shown), indicating that the effects of hSpt5 siRNA did not last indefinitely. Neither single-stranded antisense siRNA (FIG. 26A, lanes 1-7) nor mismatched duplex siRNA (FIG. 26C, lanes 15-21) affected hSpt5 proteins levels throughout the duration of the time course. These results indicated that the kinetics of hSpt5 knockdown were specific to duplex siRNA targeting hSpt5. These data also indicate that the effect of siRNA on Spt5 is relatively long-lasting, persisting at least 66 hours.

### Example 21

### Specific Silencing of Spt5 by siRNA at the mRNA Level

To determine the specificity of Spt5 knockdown by siRNA at the mRNA level, RT-PCR is used to reveal the effect of siRNA on the level of mRNA involved in Spt5 expression. Briefly, HeLa cells are transfected with Spt5 ds siRNA, harvested at various times after transfection and mRNAs are extracted. One-step RT-PCR is performed, using specific primers for Spt5 amplification. A control is run concurrently using primers specific for another, unrelated gene, e.g., CDK9, CycT1, or actin. RT-PCR products are resolved in 1% agarose gel and viewed by ethidium bromide staining. Changes in Spt5 mRNA levels with time, while the levels of mRNA of the unrelated gene remain unaltered, indicate that the effect of the siRNA is specific.

### Example 22

### Viability of Human Cells with Spt5 Knockdown

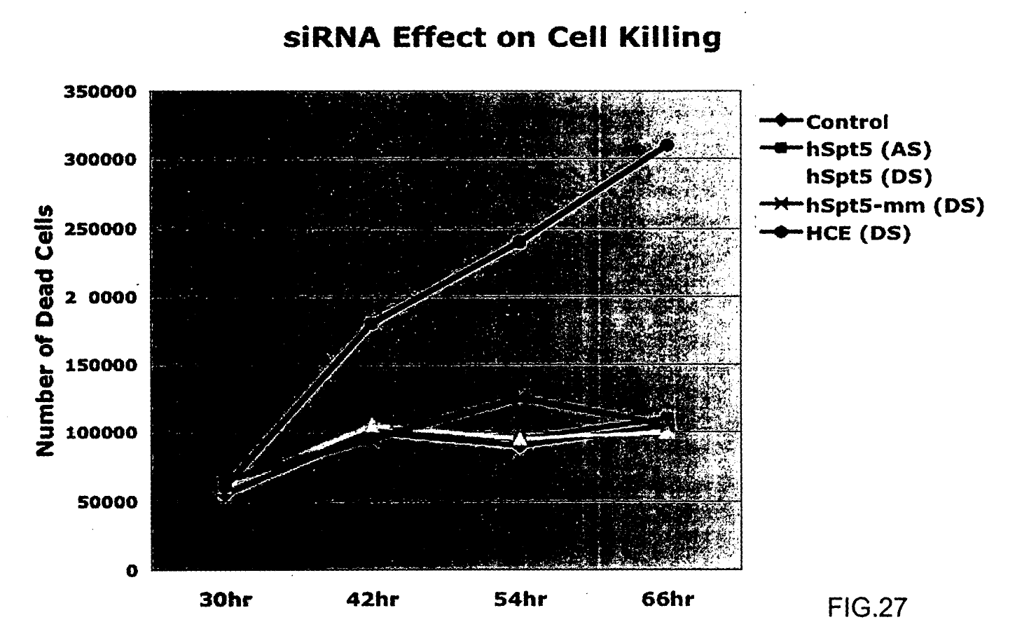

Cellular viability under various siRNA treatments was analyzed by trypan blue exclusion. Knowing that the kinetics of hSpt5 peaked at 42-54 h post-transfection, the viability of cells during an hSpt5 knockdown time course experiment could be evaluated. Cell viability was assessed using trypan blue exclusion at various times after transfection of various siRNAs. As shown in FIG. 27, during the 66 h time course experiment, the number of non-viable hSpt5 knockdown cells (yellow line) observed was comparable to mock-treated cells (no siRNA; dark blue line). Cells transfected with single-stranded antisense hSpt5 siRNA (pink line) or mismatched hSpt5 duplex siRNA (light blue line) that did not show hSpt5 knockdown also showed minimal changes in cell viability. The positive control for this experiment was human capping enzyme (HCE), which is a bifunctional triphophsatase-guanylyltransferase required for capping mRNA (reviewed in Bentley et al., 2002 Curr Opin Cell Biol 14:336-342). HCE is very likely to be essential for cell viability as the HCE homolog cel-1 in C. elegans is essential (Srinivasan et al., 2003 J Biol Chem 278:14168-14173). In contrast to hSpt5 knockdown cells, HCE knockdown cells showed a significant increase in cell death (FIG. 27, red line) over the course of the knockdown experiment. These results indicated that hSpt5 knockdown was not lethal to human cells, while a much more stringent requirement for HCE expression was essential for cell viability.

Cell viability in vivo under siRNA treatment can also be evaluated by fluorescence imaging. pEGFP-C1 reporter plasmid (harboring enhanced green fluorescent protein [GFP]) and siRNAs are cotransfected into HeLa cells using Lipofectamine™ . Briefly, HeLa cells are cotransfected by Lipofectamine™ with pEGFP-C1 reporter (GFP) plasmid and siRNAs. In general, four siRNA duplexes, including a control duplex targeting RFP and duplexes targeting Spt5 are used in these experiments. Reporter gene expression is monitored at 50 hours post transfection by fluorescence imaging in living cells. Cellular shape and density are recorded by phase contrast microscopy.

### Example 23

### hSpt5 RNAi Inhibits HIV-1 Tat Transactivation in Human Cells

A dominant paradigm for Tat up-regulation of HIV gene expression at the level of transcription elongation revolves around the ability of the Tat-TAR RNA complex to bind to P-TEFb and stimulate phosphorylation of the CTD and Spt5, thereby overriding the elongation arrest elicited by DSIF and NELF (Ping and Rana (2001), supra; Price (2000), supra).

To examine whether hSpt5 was required for HIV-1 Tat transactivation in vivo, Tat transactivation during hSpt5 knockdown in Magi cells was monitored. Magi cells are a HeLa cell line harboring a stably integrated single copy of the HIV-1 5′ LTR-β-galactosidase gene. These cells are also genetically programmed to express the CD4 receptor as well as CCR5 coreceptor for HIV-1 infection (Kimpton and Emerman, 1992 J Virol 66:2232-2239); see below). In this experiment, Magi cells were co-transfected with Tat expression plasmid pTat-RFP and hSpt5 duplex siRNA. Co-transfection with Tat siRNA was used as a positive control for inhibition of Tat transactivation while single-stranded antisense hSpt5 siRNA and mismatched siRNA were used as negative controls. Tat transactivation and protein levels were evaluated by harvesting cells 48 h post transfection, which was within the timeframe that hSpt5 knockdown peaked. Expression of HIV-1 Tat-RFP under the control of the CMV early promoter was confirmed by western blot using anti-RFP antibody and RFP fluorescence measurement on a fluorescence spectrophotometer (data not shown). In addition, immunoblot analysis confirmed that hSpt5 siRNA specifically inhibited hSpt5 protein expression in the absence and presence of HIV-1 Tat protein in Magi cells (data not shown).

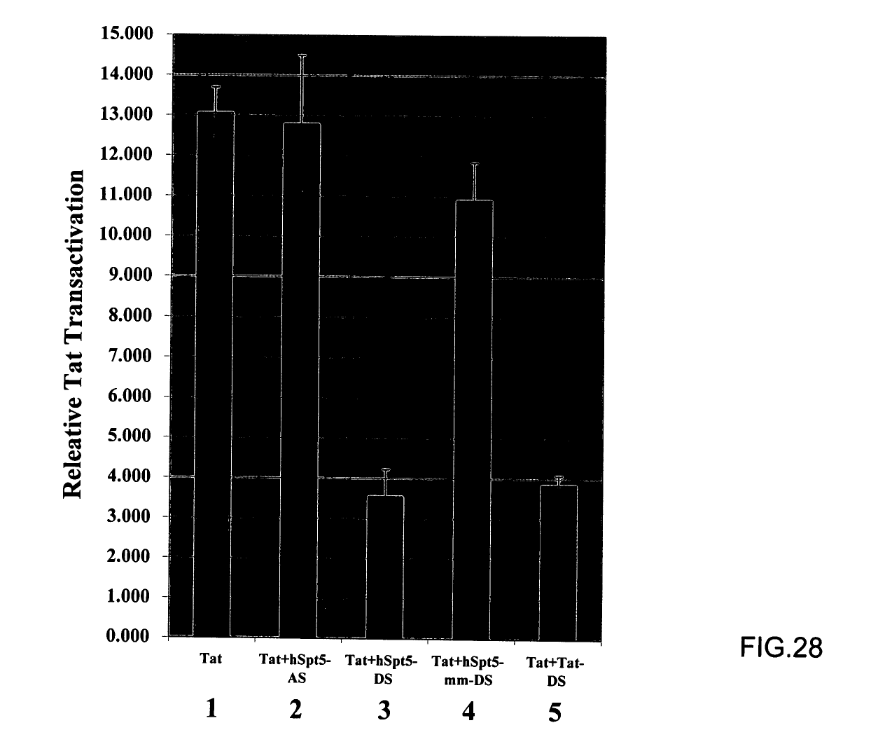

Tat-RFP enhances the expression of genes that are under the control of the HIV-1 5′ LTR promoter. In this experiment, Tat transactivation was measured by assaying the β-galactosidase activity resulting from expression of the β-galactosidase gene under the HIV-1 5′ LTR promoter. To quantify the effects of various siRNAs on HIV-1 Tat transactivation, the ratio between β-galactosidase activity in cells transfected with pTat-RFP (with or without siRNAs) and mock-treated cells not transfected with pTat-RFP was determined. The results of this quantitation are shown in FIG. 28. In Magi cells, Tat-RFP strongly stimulates the expression of β-galactosidase, represented by a 13-fold increase in Tat transactivation (FIG. 28, lane 1). On the other hand, Tat transactivation was strongly inhibited in cells transfected with Tat siRNA (FIG. 28, lane 5), as previously shown (Surabhi and Gaynor 2002 J Virol 76:12963-12973). Tat transactivation was similarly inhibited when cells were transfected with hSpt5 duplex siRNA, exhibiting only ˜30% of the Tat transactivation observed with Tat-RFP alone (FIG. 28, lane 3). Neither antisense hSpt5 siRNA nor mismatched hSpt5 siRNA (FIG. 28, lane 4) showed any effect on Tat transactivation. These results indicated hSpt5 knockdown caused by siRNA specifically targeting hSpt5 mRNA inhibited HIV-1 Tat transactivation in human cells. These results strongly supported an important role for hSpt5 in Tat transactivation in vivo and suggested that RNAi of hSpt5 had the potential to inhibit HIV-1 replication.

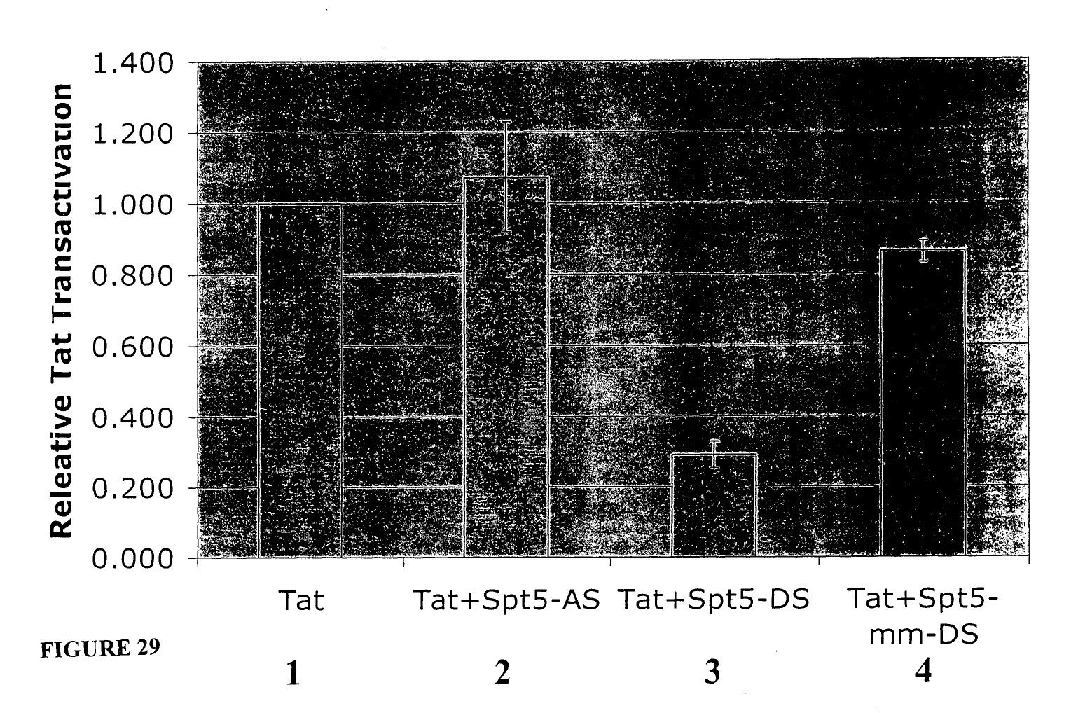

A separate experiment confirmed the results presented in FIG. 28. Tat-RFP strongly enhanced expression of genes that are under the control of the HIV-1 LTR promoter in transfected Magi cells. Tat transactivation was determined by calculating the ratio of β-galactosidase activity in pTat-RFP transfected cells to the activity in cells without pTat-RFP treatment. Inhibitory activity was determined by normalizing Tat-transactivation activity to the amount of Tat-RFP protein (represented by RFP fluorescence intensity as described in Experimental Procedures) in the presence and absence of siRNA targeting Spt5. Briefly, twenty-four hours after pre-treating Magi cells with siRNA targeting a TEF, the cells were cotransfected with pTat-RFP plasmid and additional siRNA targeting the TEF. Cells were harvested 48 hours post pTat-RFP transfection, and activity of [symbol] -galactosidase in clear cell lysates was measured (see Experimental Procedures). Tat-transactivation activity in Tat-RFP transfected cells is shown in FIG. 29, bar 1. Magi cells were cotransfected with ds siRNAs targeting Spt5 (FIG. 29, bar 3), with antisense (AS) RNA strands (FIG. 29, bar 2), or mutant (mm) siRNAs (FIG. 29, bar 4). Means±SD of two experiments are shown. Under standard experimental conditions, Tat-RFP enhanced gene transactivation 20- to 25-fold (FIG. 29, bar 1). This activation was strongly inhibited by cotransfecting host Magi cells with the specific ds siRNAs targeting Spt5 (FIG. 29, bar 3), but not with antisense (as) RNA strands (FIG. 29, bar 2), or mutant (mm) siRNAs (FIG. 29, bar 4). From these results, it can be concluded that siRNA targeting hSpt5 can inhibit Tat-transactivation in human cells without affecting cellular viability, thus making siRNA targeting hSpt5 an excellent candidate for treatment of patients infected with HIV.

### Example 24

### hSpt5 siRNAs Inhibit hSpt5 Protein Expression in the Presence or Absence of Tat Expression

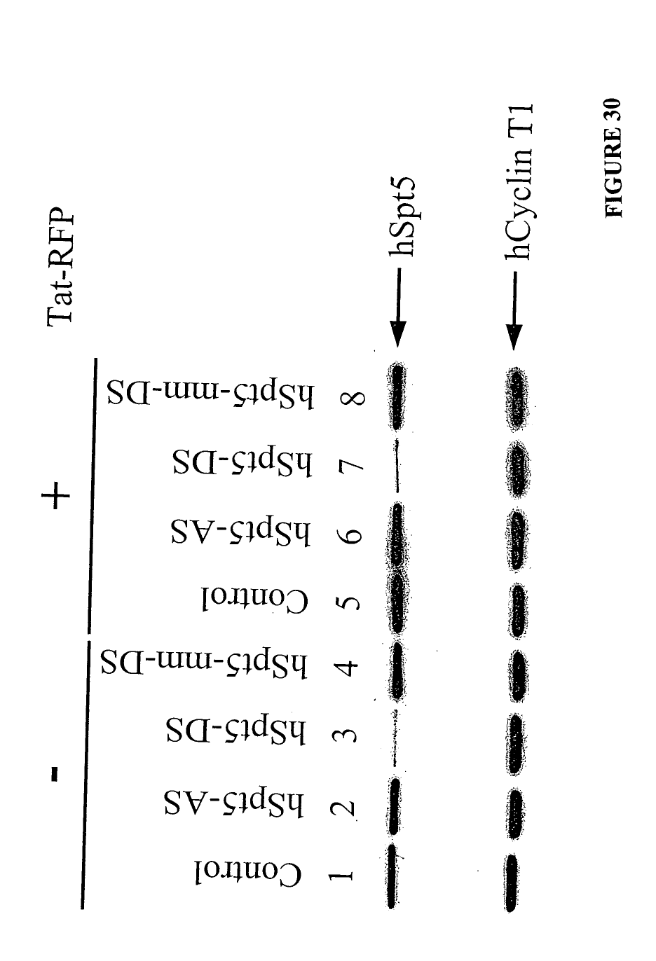

Specific RNA interference with Spt5 expression in Magi cells was demonstrated by Western blot analysis. Briefly, Magi cells were co-transfected with pTat-RFP plasmid and various siRNAs. Cells were harvested at 48 hours post-transfection, resolved on 10% SDS-PAGE, transferred onto PVDF membranes, and immunoblotted with antibodies against Spt5 (FIG. 30, upper row) or hCycT1 (FIG. 30, lower row). RNAi activities in Magi cells treated with antisense (AS) strands of Spt5 siRNAs are shown in FIG. 30, lanes 2 and 7, while those of cells treated with ds siRNA targeting Spt5 are shown in FIG. 30, lanes 3 and 7. RNAi activities in cells treated with mismatch Spt5 (hCycT1. mm) siRNAs with two mismatches are shown in FIG. 30, lanes 4 and 8.

From these results, it can be concluded that siRNA targeting hSpt5 can inhibit hSpt5 protein expression in the presence or absence of Tat protein, making siRNA targeting hSpt5 an excellent candidate compound for treatment of patients infected with HIV.

### Example 25

### RNAi Inhibition of HIV-1 Infectivity

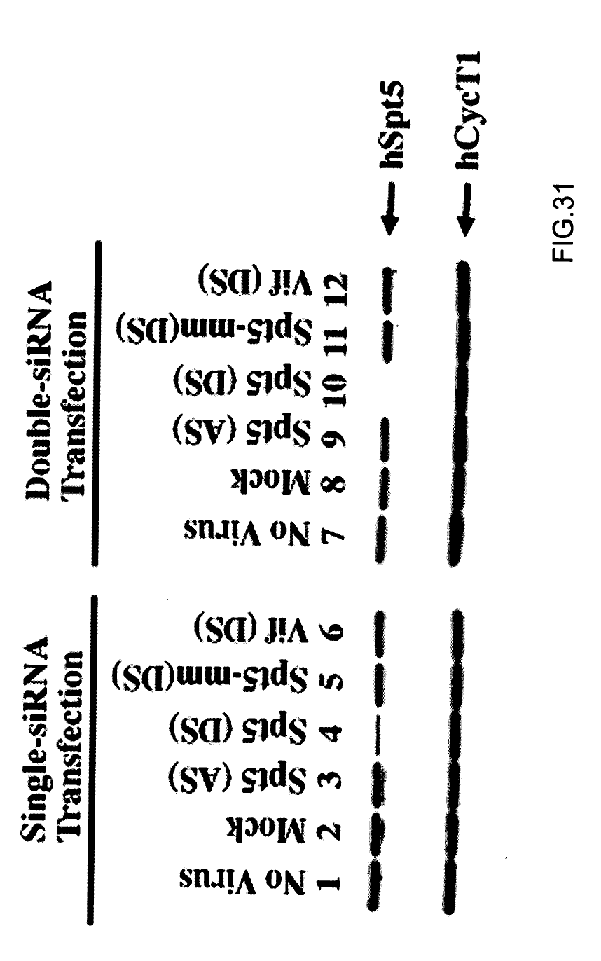

Since hSpt5 knockdown effectively inhibited Tat transactivation, we next determined whether hSpt5 knockdown could inhibit HIV-1 replication. To evaluate the effect of hSpt5 knockdown on HIV-1 replication, a double siRNA transfection protocol was used to maximize the knockdown efficiency of hSpt5 during HIV-1 infection. Magi cells were transfected with siRNA directed against hSpt5. Cells mock transfected without siRNA, or transfected with single-stranded antisense hSpt5 siRNA or mismatch hSpt5 siRNA were used as negative controls. Transfection with Nef siRNA was used as a positive control. 24 h after the first transfection, a second siRNA transfection was performed. 24 h later, doubly transfected cells were infected with various concentrations of HIV_{NL-GFP}, an infectious molecular clone of HIV-1. Knockdown of hSpt5 protein levels was then evaluated 48 h post infection in doubly transfected cells. An even larger decrease in hSpt5 protein levels was observed in doubly transfected cells as compared to singly transfected cells (FIG. 31, compare lanes 4 and 10), suggesting that more robust knockdown of gene expression can be achieved using this double transfection method.

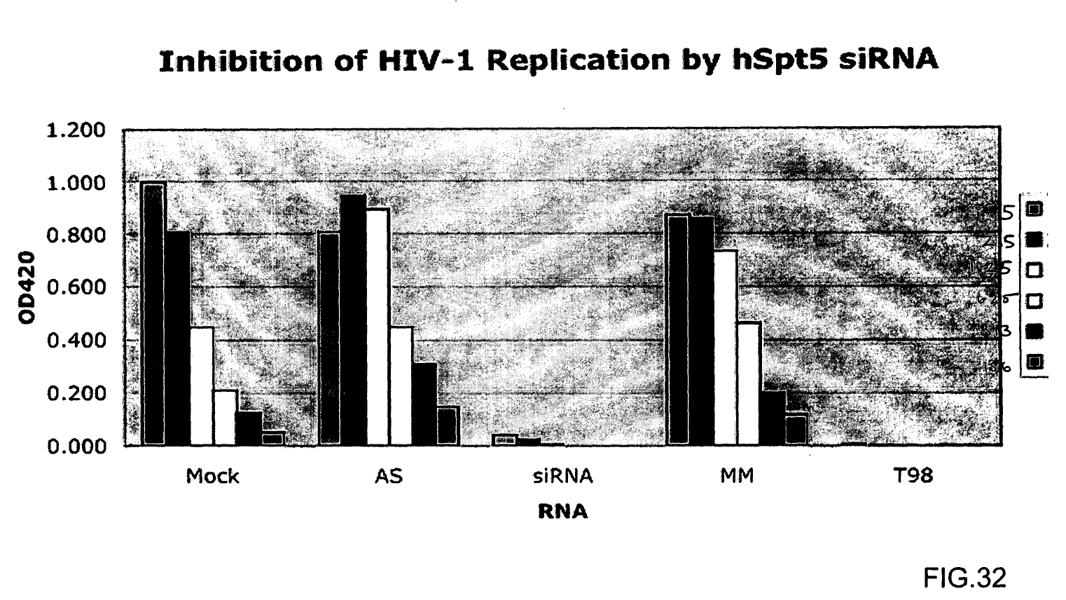

HIV-1 Tat-mediated transactivation of the 5′ LTR occurring in cells infected with virus led to β-galactosidase production, which was also quantified 48 h post-infection. In this single-cycle replication assay for evaluating HIV-1 replication, β-gal activity reflected the activity of reverse transcriptase and viral replication of varying amounts of viral inoculum. Therefore, changes in β-gal activity could be directly correlated to changes in the efficacy of HIV replication. The positive siRNA control targeting HIV Nef showed decreased levels of β-gal activity and viral infectivity, as shown previously (FIG. 32; (Jacque et al., 2002 Nature 418:435-438). Double-stranded siRNA directed against hSpt5 showed a similar decrease in β-gal activity when compared with Nef knockdown. This observed decrease was equivalent to the β-gal activity measured when using 32 times less viral inoculum with mock-treated cells (FIG. 32), indicating that hSpt5 knockdown had significantly reduced HIV replication. Control experiments using hSpt5 single-stranded antisense or mismatched duplex siRNA duplexes showed no antiviral activities. In addition, no significant toxicity or cell death was observed during these experiments, suggesting that hSpt5 knockdown was not lethal even in the context of HIV- 1 infection. These results demonstrated that HIV replication was modulated by siRNAs targeting hSpt5, further establishing an important role for hSpt5 in Tat transactivation and HIV-1 replication in vivo.

### Example 26

### hSpt5 is Required for Upregulation of Heat Shock Genes During Heat Shock

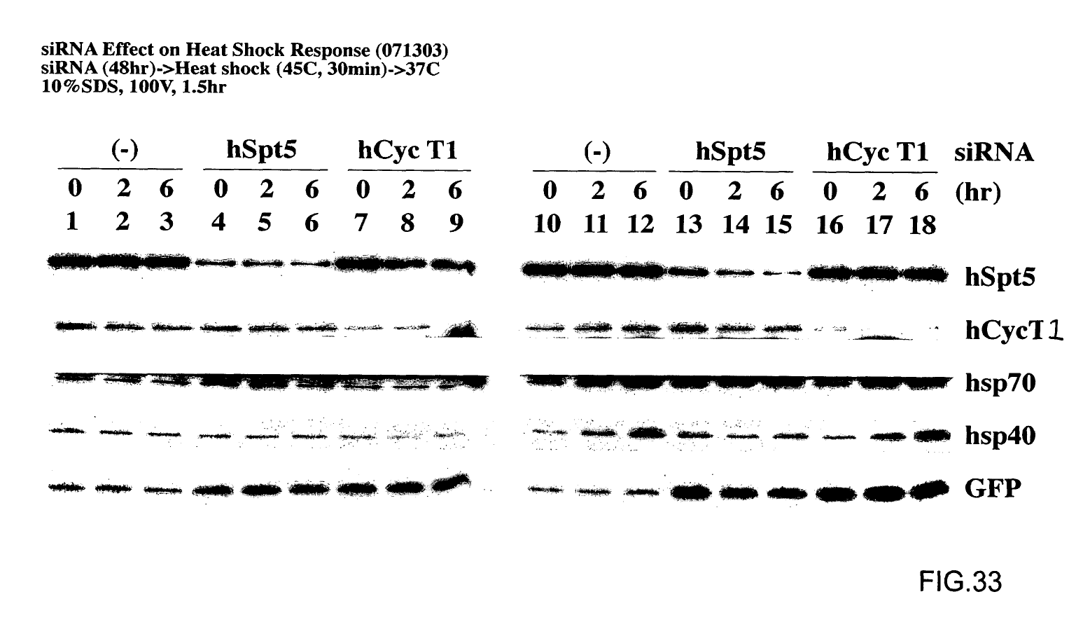

Homologs of hSpt5 were previously linked to heat shock response in D. melanogaster and C. elegans (Andrulis et al., 2000 Genes Dev 14:2635-2649; Wu et al., 2003 Genes Dev 17:1402-1414; Shim et al., 2002 Genes Dev 16, 2135-2146), suggesting that hSpt5 may have a conserved role in regulating heat shock genes. To further define hSpt5 cellular function and define a potential role for hSpt5 in heat shock response in human cells, the effect of hSpt5 knockdown on heat shock genes Hsp40 and Hsp70 was evaluated in Magi cells. Cells were transfected without or with hSpt5 duplex siRNA, and 48 h after transfection, cells were incubated under heat shock conditions at 45° C. for 30 min. Cells were then harvested at various time points after heat shock and cell lysates were evaluated for protein levels by immunoblot analysis with hSpt5, Hsp40, Hsp70 and hCycT1 antibodies. As shown in FIG. 33, cells without hSpt5 siRNA did not exhibit any decrease in hSpt5 protein levels after heat shock while Hsp40 and Hsp70 showed an increase in protein levels post-heat shock (compare lanes 1-3 to lanes 4-6), as seen previously. By contrast, in cells exhibiting knockdown of hSpt5 protein levels even after heat shock, Hsp40 and Hsp70 no longer showed the upregulation of expression seen in cells without siRNA during recovery from heat shock (FIG. 33, compare lanes 10-12 to lanes 13-15). hCycT1, used an internal control for specificity of hSpt5 knockdown and effects on upregulation of heat shock genes, showed no change in protein levels after heat shock whether cells were transfected with or without hSpt5 siRNA (Lanes 1-6, 10-15). These results indicated that hSpt5 was important for specifically regulating Hsp40 and Hsp70 during heat shock response, raising the possibility that hSpt5 may be a general regulatory factor involved in stress responses in human cells.

### Discussion of Examples 19-26

The role of hSpt5 in Tat transactivation has been predominantly analyzed using in vitro assays that have led to conflicting ideas about hSpt5 function during HIV-1 replication. hSpt5, as part of the DSIF complex, was originally discovered as a negative elongation factor required for conferring DRB sensitivity to transcription elongation complexes thereby inhibiting transcription (Wada et al., 1998 Genes Dev 12:343-356). This negative barrier provided by hSpt5 was thought to be relieved through P-TEFb phosphorylation of both hSpt5 and RNA pol II CTD, resulting in increased processivity of RNA pol II complexes (Renner et al., 2001 J Biol Chem 276:42601-42609; Wada et al., 1998 Genes Dev 12:343-356; Yamaguchi et al., 1999 Cell 97:41-51; Price 2000 Mol Cell Biol 12:2078-2090; Isel and Karm 1999 J Mol Biol 290:929-941; Ivanov et al., 2000 Mol Cell Biol 20:2970-2983; Ping and Rana 2001 J Biol Chem 276:12951-12958; Kim et al., 2002 Mol Cell Biol 22:4622-4637; Zhou et al., 2000 Mol Cell Biol 20:5077-5086; Zhou et al., 1998 EMBO J 17:3681-3691; Wu-Baer et al., 1998 J Mol Biol 277:179-197). Increased processivity has also been linked to the phosphorylated form of hSpt5 conferring a positive effect on transcription elongation (Wu-Baer et al., 1998 J Mol Biol 277:179-197; Kim et al., 1999 Mol Cell Biol 19:5960-5968; Ping and Rana 2001 J Biol Chem 276:12951-12958). Evidence for Spt5 as a positive regulator of transcription elongation has been documented across several organisms, including yeast, C. elegans, zebrafish, and humans. In humans, hSpt5 was found to be a positive regulatory factor involved in HIV-1 Tat transactivation and has a specific role in antitermination (Kim et al., 1999 Mol Cell Biol 19:5960-5968; Wu-Baer et al., 1998 J Mol Biol 277:179-197; Ping and Rana 2001 J Biol Chem 276:12951-12958; Bourgeois et al., 2002 Mol Cell Biol 22:1079-1093). On the contrary, however, it has also been shown that Tat is able to enhance transcription elongation in vitro in the absence of hSpt5. These results appeared to indicate that P-TEFb phosphorylation of RNA pol II was the sole event that directly led to Tat transactivation and increased RNA pol II processivity (Kim 2002, ibid). Thus, collectively, the requirement for hSpt5 in positively regulating transcription elongation during Tat transactivation has remained ambiguous from results of in vitro studies.

Herein, we studied the role of hSpt5 in vivo using RNAi and established that hSpt5 does have a required positive role in Tat transactivation and HIV-1 replication. Knockdown of hSpt5 gave insight into several functional aspects of the hSpt5 protein. First, knockdown of hSpt5 was not lethal in Magi cells, indicating that hSpt5 was not required for cell viability. This was an interesting result in that studies of Spt5 mutants in yeast and zebrafish and RNAi of Spt5 in C. elegans have shown that Spt5 was essential for growth and/or embryonic development in those organisms (Guo et al., 2000 Nature 408:366-369; Hartzog et al., 1998 Genes Dev 12:357-369; Shim et al. 2002 ibid). It seems likely that hSpt5 holds similar essential fuinctions in human cells during embryonic development but may not be required in adult cells. Alternatively, hSpt5 knockdown may have led to decreased levels of expression that were still sufficient for hSpt5 to carry out its essential functions. In either case, our results do support further study of RNAi of hSpt5 as a potential therapeutic strategy for fighting HIV-1 infection since there is the potential that HIV- 1 functions could be targeted for inhibition without interfering with host cell functions.

The key finding of this study was that hSpt5 knockdown significantly inhibited both Tat transactivation and HIV-1 replication. These results indicated that hSpt5 was a bona fide regulator of Tat transactivation that is required for HIV-1 replication in vivo. Having established hSpt5 importance in HIV-1 replication, it is interesting to dissect how the conflicting in vitro results regarding hSpt5 role in Tat transactivation connect to what we observed with hSpt5 knockdown. Our in vivo results strongly support those in vitro assays recapitulating Tat transactivation that showed immunodepletion of hSpt5 significantly inhibited Tat transactivation (Kim et al., 1999 ibid; Wu-Baer et al., 1998 ibid). More complicated to tie into these and our in vivo results are the in vitro assays showing that P-TEFb hyperphosphorylation of the CTD in the absence of hSpt5 still enhanced RNA pol II processivity during Tat transactivation (Kim et al., 2002 ibid).

In reviewing the work of Kim et al., it was well documented that hSpt5 was not required for hyperphosphorylation of RNA pol II during Tat transactivation. However, the processivity of RNA pol II complexes during Tat transactivation in the absence of hSpt5 was not as clear. Although complete inhibition of Tat transactivation was not observed, a decrease in transcriptional processivity was still apparent when hSpt5 was immunodepleted in their transcriptional assay. In reconciling whether P-TEFb hyperphosphorylation was directly required for Tat transactivation to the exclusion of hSpt5, we would like to propose that the required function of P-TEFb hyperphosphorylation may be distinct from the role hSpt5 plays in enhancing RNA pol II processivity during Tat transactivation. In our model, P-TEFb hyperphosphorylation would occur first, triggering enhanced processivity of RNA pol II. hSpt5 presumably is phosphorylated at around the same time as RNA pol II, stimulating hSpt5 to switch from a negative regulator to a positive elongation factor. Phosphorylated hSpt5 may then be important for stabilizing the conformation of processive RNA pol II complexes formed during the initial step of Tat transactivation.

Conceivably, hSpt5 key function in transcription elongation is as a stabilization factor that reinforces the processivity conformation of RNA pol II complexes that were first formed after P-TEFb hyperphosphorylation of the CTD. This type of role would also support hSpt5 function as an antiterminator factor that was described previously (Bourgeois, 2002 ibid). If our model for hSpt5 function proves correct, then the in vitro assays showing that P-TEFb hyperphosphorylation was the only requirement for enhanced processivity may have recapitulated conditions that allowed for maintenance of stable RNA pol II processive complexes in the absence of hSpt5 but were not wholly representative of intracellular conditions. In other words, the hyperphosphorylated RNA pol II complexes remained stably processive in the context of the particular in vitro conditions used in those assays. However, under in vivo conditions, RNA pol II complexes, after being hyperphosphorylated by P-TEFb, would require hSpt5 for stabilization of processive RNA pol II complexes. Overall, the in vitro and in vivo approaches taken to address the importance of hSpt5 function all shed light on the complex, multi-faceted nature of Tat transactivation. Accordingly, these studies altogether support important roles for both P-TEFb and hSpt5 in mediating transcription elongation during HIV-1 replication in vivo.

Further analysis of cells with knocked down hSpt5 showed that heat shock genes Hsp70 and Hsp40 required hSpt5 for increased expression in response to heat shock. These results provided a functional role for hSpt5 in adult human cells and suggested that hSpt5 was important for upregulating the expression of genes in response to stress. Interestingly, hSpt5 has been previously implicated in heat shock gene regulation in other organisms, including D. melanogaster and C. elegans, suggesting that regulation of heat shock genes by hSpt5 may be a generally conserved mechanism among eukaryotes. In flies, hSpt5 was localized to heat shock gene loci upon heat shock (Andrulis et al., 2000 ibid; Wu et al. 2003 ibid) suggesting a transcription regulation role for hSpt5 in heat shock responses. In C. elegans, knockdown of hSpt4, hSpt5 and P-TEFb resulted in altered heat shock gene expression of Hsp70 (Shim et al., 2002 ibid). It is worth speculating whether the role of hSpt5 in regulating heat shock genes is similar to the role it plays in Tat transactivation, and/or if HIV-1 infection stimulates a general shift in gene regulation that is mediated by hSpt5 and P-TEFb. Previous studies have shown that expression from the 5′LTR displays similar kinetics of upregulation during heat shock as heat shock response genes like Hsp70 (Kretz-Remy and Arrigo 1994 FEBS Lett 351:191-196). This raises the possibility that the mechanism of Tat transactivation mirrors how heat shock genes are upregulated during heat shock. Future analysis of hSpt5 role in regulating heat shock genes and Tat transactivation should further elucidate such connections between regulation of the 5′LTR during HIV-1 infection and heat shock gene expression.

The dual function of hSpt5 as a negative and positive transcription elongation factor demonstrates the complexity associated with transcriptional regulation during transcription elongation and HIV-1 Tat transactivation. The results presented here firmly establish hSpt5 as important for Tat transactivation and HIV-1 replication, compellingly tying together previous studies that sought to decipher the intriguing role this factor plays in these processes. This analysis also exemplified the benefits of using RNAi to study hSpt5 function in vivo. Building on these studies should prove useful for further defining the intricate mechanisms associated with hSpt5 cellular functions generally and during the course of HIV-1 infection.

### Example 27

### Genome-Wide Analysis of Gene Expression in TEF Knockdown HeLa Cells

To investigate the effects of TEF knockdown on global gene expression, total mRNA is isolated from HeLa cells treated with and without siRNA directed against one or more TEF and the expression of various genes is analyzed using the GeneChip® Human Genome U133 (HG-U133) from Affymetrix. The HG- includes HG-133A and HG-133B sets of arrays containing 22,283 and 22,645 genes, respectively. Another 100 human maintenance genes on both arrays serve as a tool to normalize and scale the data prior to making comparisons.

Briefly, HeLa cells are treated with and without ds siRNA directed against one or more TEF, e.g. Spt4, Spt5, and/or Spt6, and total mRNA is isolated. Total mRNA is then used to synthesize ds cDNAs, from which biotin-labeled cRNA was synthesized and fragmented. Fragmented cRNA was then subjected to high-density oligonucleotide microarray hybridization (GeneChip®) using Human Genome U133 from Affymetrix (see Experimental Procedures).

This procedure was used to identify genes whose expression is affected by knock down of P-TEFb, a positive transcription elongation factor (see Examples 8-14 supra). HeLa cells were treated with ds siRNAs targeting CDK9 or CycT1, as described in U.S. Provisional Patent Application No. 60/423,198 to the present inventor. Of the 44,928 genes expressed, 90 are displayed in FIG. 12 and FIG. 13 by class, based on their putative functions. Each row represents one gene. Column 1 of FIG. 12 and FIG. 13 indicates hCycT1 ds siRNA treatment and column 2 indicates CDK9 ds siRNA treatment. The down—(FIG. 12) and up—(FIG. 13) regulated genes are represented by green and red, respectively. The brightness of each color reflects the magnitude of the gene expression level (Signal Log Ratio). See Experimental Procedures for details of analysis.

Genome-wide analysis of the gene expression profile of P-TEFb knockdown cells revealed that 201 genes out of 44,928 were down-regulated. A complete list of down- and up-regulated genes with names and accession numbers can be found in Table 1. Of those 201 genes, 53 are known and can be classified according to their function or protein product activity. It is widely accepted that P-TEFb is a positive transcription factor during RNA pol II elongation. Down-regulated genes in the P-TEFb knockdown background are presumably those required by P-TEFb for normal levels of expression, especially at the transcription elongation level. These results indicate that P-TEFb regulation of various cellular genes is affected by siRNA targeting either CDK9 or CycT 1. Because P-TEFb works cooperatively with other TEFs, e.g. Spt4, Spt5, and/or Spt6, the same genes are likely to be regulated by other TEFs, e.g. Spt4, Spt5, and/or Spt6.

P-TEFb is Required for Embryonic Transcription but is Relatively Non-essential in Adults

Broadly speaking, about 50% (25 out of 53) of the known P-TEFb-down-regulated genes are known or likely to be involved in controlling and mediating cell proliferation and differentiation. These genes can be further divided into three classes (FIG. 12, I-III). The first class (FIG. 12, I) includes genes directly linked to cellular proliferation and differentiation. Most of these genes belong to the protein tyrosine kinase (PTK) superfamily. PTKs catalyze phosphate transfer from ATP to tyrosine residues on protein substrates, activating numerous signaling pathways leading to cell proliferation, differentiation, migration, or metabolic changes and playing a prominent role in the control of a variety of cellular processes during embryonic development (Hubbard and Till, Annual Review of Biochemistry 69:373-398 (2000)). Two classes of PTKs are affected by P-TEFb knockdown: transmembrane receptor protein tyrosine kinases (RTKs) and non-receptor tyrosine kinases (NRTKs). AXL receptor tyrosine kinase (AXL), discoidin domain receptor 1 (DDR1), epidermal growth factor receptor (EGFR), and fibroblast growth factor receptor (FGFR) belong to the RTKs, while cell adhesion kinase (CAK) belongs to the NRTKs. AXL, DDR1 and EGFR are required for the epithelial-mesenchymal transition during embryonic development, which allows multicellular organisms to get past the blastula stage (Thiery, Nature Reviews Cancer 2:442-454 (2002)).

Transforming growth factor beta (TGF-beta), also down-regulated by P-TEFb knockdown (FIG. 12, I), binds to another membrane receptor family with diverse functions during embryonic development and adult tissue homeostasis (Attisano and Wrana, Science 296:1646-1647 (2002); Massague, Nature Reviews Molecular Cell Biology 1:169-178 (2000)). The genes for pre-T/NK cell-associated proteins (fasciculation and elongation protein zeta 2) are preferentially expressed in early stages of human T/NK cells and brain, suggesting that they play a role in early development (Ishii et al., Proc. Natl. Acad. Sci. USA 96:3928-3933 (1999); Whitehouse et al., Eur. J. Biochem. 269:538-545 (2002)). Brain-derived neurotrophic factor (BDNF) has been implicated in activity-dependent plasticity of neuronal function and network arrangement (Yamada et al., Journal of Neuroscience 22:7580-7585 (2002)).

A second class of genes affected by P-TEFb knockdown is functionally linked to the cell membrane and extracellular matrix (FIG. 12, II). Junction plakoglobulin (JUP) cooperates with beta-catenin and promotes epithelial growth and morphogenesis (Hatsell and Cowin, Nat. Cell Biol. 3:E270-272 (2001)). Moesin (MSN), a plasma membrane protein associated with the underlying cytoskeleton, determines cell shape and participates in adhesion, motility and signal transduction pathways (Bretscher et al., Nature Reviews Molecular Cell Biology 3:586-599 (2002)). This class of genes is also required for embryonic development, especially for angiogenesis and neuronal development. For example, integrin functions in epithelial cell organization and synaptogenesis during development of the CNS (Hynes, Nature Medicine 8:918-921 (2002); Milner and Campbell, J. Neuroscience Research 69:286-291 (2002)). Epithelial membrane protein 1 (EMP1), which is highly expressed by immature neurons in the embryonic stage, functions in neuronal differentiation and neurite outgrowth (Wulf and Suter, Brain. Res. Dev. Brain Res. 116:169-180 (1999)).

A third class of genes is involved in signal transduction (FIG. 12, III), the events downstream of membrane receptor activation. The inositol 1,4,5-triphosphate receptor (ITPR1) responds to G-protein-coupled receptor activation (Katso et al., Annual Review of Cell & Developmental Biology 17:615-675 (2001)), while mitogen-activated protein kinase 6 (MAPK6) propagates cell proliferation/differentiation signals from receptor tyrosine kinase activation (Hubbard and Till, (2000), supra; Katso et al., (2001), supra). Non-receptor type protein tyrosine phosphatase regulates phosphotyrosine signalling events during complex ectodermal-mesenchymal interactions that regulate mammalian limb development (Arregui et al., Neurochemical Research 25:95-105 (2000); Saxton et al., Nat. Genet. 24:420-423 (2000)). Protein phosphatase 1 (PPP1CB) regulates the phosphorylation status of anti-apoptotic and pro-apoptotic proteins and their cellular activity in the apoptosis cascade (Klumpp and Krieglstein, Curr. Opin. Pharmacol. 2:458-462 (2002)). Dual specificity phosphatase 2 (DUSP2) participates in the regulation of intracellular signal transduction mediated by MAP kinases (Yi et al., Genomics 28:92-96 (1995)).

There is cross talk among these three gene classes (FIG. 12, I, II, and III). For example, collagen (type III, alpha 1), an extracellular matrix membrane protein can be a direct ligand for tyrosine kinase receptors (class I) and integrin (class II) (Shrivastava et al., Mol. Cell. 1:25-34 (1997)). Coordinated regulation of PTK (class I) and protein phosphatase (class II) also acts at different steps in a common signal transduction process (Bauman and Scott, Nature Cell Biology 4:E203-206 (2002)). Non-receptor type protein tyrosine phosphatase (class III) positively regulates BDNF-promoted (class I) survival of ventral mesencephalic dopaminergic neurons (Takai et al., J. Neurochem. 82:353-364 (2002)). FGFR (class I), EMP I (class II), pre-T/NK cell-associated protein (fasciculation and elongation protein zeta 2; class I) and integrin (class II) all participate in neuronal development. Nedasin (S form, class X), which is predominantly expressed in the brain, plays a role in the formation and structural changes of synapses during neuronal development by modifying clustering of neurotransmitter receptors at synaptic sites (Kuwahara et al., J. Biol. Chem. 274:32204-32214 (1999)). Down-regulation of the genes coding for these last five proteins by P-TEFb knockdown indicates an important role for P-TEFb during neuronal development.

From the expression profile of these three classes of genes, it can be concluded that P-TEFb is essential for embryonic gene expression and development, while knockdown of its subunits (hCycT1 and CDK9) does not affect cellular viability at the adult stage. It has been proposed that the P-TEFb complex is required for global gene expression during embryonic development of C. elegans (Shim et al., Genes Dev. 16:2135-2146 (2002)). Knockdown of CDK9 or CycT1 siRNA in C. elegans embryos inhibits transcription of embryonic genes, including the MAP kinase pathway and cell cycle-related genes (Shim et al. (2002), supra). The non-essential nature of P-TEFb in adult tissues makes it an ideal therapeutic target for treatment of HIV/AIDS and disorders characterized by unwanted cellular proliferation using the methods of the present invention.

The Role of TEFs in Human Cancers

An intriguing finding is that genes linked to embryonic development and showing down-regulation in P-TEFb knockdown cells (as described above) also participate in tumorogenesis and metastasis. Dysfunction of protein tyrosine kinases (FIG. 12, I) or aberrations in key components of the signaling pathways they activate can lead to severe pathologies such as cancer, diabetes, and cardiovascular disease. For example, overexpression of EGFR (FIG. 12, I) has been implicated in mammary carcinomas, squamous carcinomas and glioblastomas (Schlessinger, Cell 110:669 (2002)). AXL, another receptor tyrosine kinase (FIG. 12, I), was originally identified with oncogenic potential and transforming activity in myeloid leukemia cells(Burchert et al., Oncogene 16:3177-3187 (1998)). Elevated TGF-beta levels can contribute to tumor progression and metastasis (Attisano and Wrana (2002), supra; Massague (2000), supra). Lysyl oxidase (LOX class II), an extracellular matrix remodeling enzyme, is up-regulated in prostatic tumor, cutaneous and uveal cell lines (Kirschmann et al., Cancer Res. 62:4478-4483 (2002)). Down-regulating these genes by P-TEFb knockdown using siRNA targeting CDK9 or CycT1 thus provides a new therapeutic strategy for inhibiting tumorigenesis and metastasis.

Genes involved in mediating progression through the cell cycle and as checkpoints in cancer were regulated by P-TEFb (FIG. 12, IV). Cyclin G1 is the downstream target of the P53 pathway and plays a role in G2/M arrest, damage recovery and growth promotion after cellular stress(Kimura et al., Oncogene 20:3290-3300 (2001)). Cyclin D, a cell-cycle regulatory protein essential for G1/S transition, has been identified as a potential transforming gene in lymphoma (Motokura and Arnold, Curr. Opin. Genet. Dev. 3:5-10 (1993)). Misregulation of the activity of its partner, CDK4/6, by overexpression of Cyclin D leads to hyperproliferative defects and tumor progression (Ortega et al., Biochim. Biophys. Acta 1602:73-87 (2002)). Several marker genes in cancer cells (class V) are also regulated by P-TEFb. For example, breast cancer-specific protein 1 (BCSG1) is overexpressed in advanced, infiltrating breast cancer and colorectal tumors (Lu et al., Oncogene 20:5173-5185 (2001)). Another example is soluble urokinase plasminogen activator receptor (SUPAR), which is present in high concentrations in cystic fluid form ovarian cancer, tumor tissue of primary breast cancer, and gynecological cancer (Riisbro et al., Clin. Cancer. Res. 8:1132-1141 (2002); Wahlberg et al., Cancer Res. 58:3294-3298 (1998)). Although the functions of these marker genes are still unknown, their high correlation with cancer has been used for prognosis in cancer therapy. The down-regulation of cyclin D and cancer marker genes by P-TEFb knockdown offer more evidence to support the proposal that siRNA targeting TEFs is a new and potent method of cancer therapy.

Links Between P-TEFb and Stress Responses

Another interesting group of genes down-regulated by P-TEFb are those involved in responding to stress or oxidant-mediated regulation (FIG. 12, VII). There are three known major redox signaling systems in eukaryotic cells, namely glutathione/glutathione reductase, thioredoxin/thioredoxin reductase and glutaredoxin (GLRX, thioltransferase) (Powis et al., Pharmacology & Therapeutics 68:149-173 (1995)). SH3 domain-binding glutamine-rich 3-like protein (SH3BGRL3) also belongs to the thioredoxin family. Glutathione S-transferase M4 (GSTM4) is involved in detoxifying reactive electrophiles, such as drug or foreign compounds, by catalyzing their reaction with glutathione (GSH) (Cotgreave and Gerdes, Biochem. Biophys. Res. Commun. 242:1-9 (1998)). Oxidant-mediated regulation by GSH systems plays a direct role in cellular signaling through thiol-disulfide exchange reactions with membrane-bound receptor proteins, transcription factors, and regulatory proteins in the cell (Cotgreave and Gerdes (1998), supra). During stress responses, redox regulation has an important function in biological events such as DNA synthesis, enzyme activation, gene expression and cell cycle regulation. Down-regulation of genes involved in these events by P-TEFb knockdown in cells indicates an important role for P-TEFb in stress responses, especially oxidant-mediated regulation of cell proliferation. Moreover, glutathione (GSH) has been linked to multi-drug resistance (Cotgreave and Gerdes (1998), supra). These data provide additional evidence for P-TEFb as an effective therapeutic target in cancer therapy.

Unlike the covalent modifications in redox regulation described above, the Bcl-2-associated athanogene (BAG) family of modulating proteins (FIG. 12, VII) functions through alterations in conformation and influences signal transduction through non-covalent post-translational modifications (Takayama and Reed, Nat. Cell Biol. 3:E237-241 (2001)). The BAG family molecular chaperone regulator-2 (BAG-2) belongs to this family, which contains an evolutionarily conserved “BAG domain” that allows its members to interact with and regulate the Hsp 70 (heat shock protein 70) family of molecular chaperones (Takayama and Reed (2001), supra). Like Hsp 70, BAG-family proteins have been reported to mediate the physiological stress signaling pathway that regulates cell division, death, migration, and differentiation (Takayama and Reed (2001), supra). P-TEFb has been shown to be recruited to heat shock loci in Drosophila melongaster and to co-localize with Hsp 70 and Hsp 90 upon heat shock stress (Lis et al., Genes Dev. 14:792-803 (2000)). Down-regulation of the BAG-2 gene in P-TEFb knockdown cells indicates an important role for P-TEFb in regulating Hsp70 molecular chaperones in human cells. CDK9 itself has been proposed to form complexes with Hsp 70 and Hsp90/cdc37, thereby involving this chaperone-dependent pathway in the stabilization/folding of CDK9 as well as the assembly of an active CDK9/CycT1 complex (O'Keeffe et al., J. Biol. Chem. 275:279-287 (2000)). Thus, given this evidence of P-TEFb involvement in the stress response, down-regulation of P-TEFb by siRNA targeting Cyct1 or CDK9 has implications for the treatment of stress-related disorders, as well as aging and senescence.

Role of P-TEFb in Metabolism and Biosynthesis

Several genes involved in metabolism and biosynthesis are also regulated by P-TEFb (FIG. 12, IX). Human iron-sulfur protein or ferredoxin (FDX1) serves as an electron transport intermediary for mitochondrial cytochrome P450 involved in steroid, vitamin D, and bile acid metabolism(Lill et al., Biol. Chem. 380:1157-1166 (1999)). Low-density lipoprotein receptor-related protein 5 (LRP5) contains conserved modules characteristic of the low-density lipoprotein (LDL) receptor family, genetically associated with Type 1 diabetes (Figueroa et al., J. Histochem. Cytochem. 48:1357-1368 (2000)). Because alterations in LRP5 expression may be responsible for susceptibility to diabetes, LRP5 is therefore a potential target for therapeutic intervention. The vacuolar (H+)-ATPases (or V-ATPases) function in the acidification of intracellular compartments in eukaryotic cells. Eukaryotic translation elongation factor 1, alpha 2 isoform (EEF1A2), a key factor in protein synthesis, has been shown to have oncogenic properties: it enhances focus formation, allows anchorage-independent growth and decreases doubling time of fibroblasts (Anand et al., Nature Genetics 31:301-305 (2002)). EEF1A2 is amplified in 25% of primary ovarian tumors, its expression makes NIH3T3 fibroblasts tumourigenic and it increases the growth rate of ovarian carcinoma cells (Anand et al. (2002), supra). This is further evidence that downregulation of TEFs using siRNA is useful for treating disorders associated with unwanted cell proliferation, including cancer.

Role of P-TEFb in Cell Cycle Regulation

Also affected by P-TEFb knockdown were genes involved in cell cycle regulation (FIG. 12, VI), which in turn controls proliferation and differentiation. The retinoblastoma (RB) protein regulates both the cell cycle and tissue-specific transcription by modulating the activity of its associated factors (MacLellan et al., Mol. Cell Biol. 20:8903-8915 (2000)). Efforts to identify such cellular targets have led to the isolation of two novel proteins, RB-associated protein (RBP21) and RB- and p300-binding protein EID-1 (an E1A-like inhibitor of differentiation) (MacLellan et al. (2000), supra). Although its cellular function is still unclear, RBP21 is widely expressed in various human tissues and cancer cell lines. EID-1 is a potent inhibitor of differentiation, an activity that has been linked to its ability to inhibit p300 (and the highly related molecule, CREB-binding protein, or CBP) acetylation of histones (Miyake et al., Mol. Cell Biol. 20:8889-8902 (2000)). EID-1, which is rapidly degraded by the proteasome as cells exit the cell cycle, may act at a nodal point that couples exit from the cell cycle to transcriptional activation of genes required for differentiation (Miyake et al. (2000), supra). Regulation of EID-1 expression by P-TEFb knockdown provides evidence that P-TEFb is also involved in cell cycle regulation, especially RB-linked regulation of proliferation and differentiation. This is yet more evidence that downregulation of TEFs using siRNA is useful for treating disorders associated with unwanted cell proliferation, including cancer.

Up-Regulated Genes

Four major classes of up-regulated genes were observed, including those involved in signal transduction (FIG. 13, I), transcription regulation (FIG. 13, II), cell cycle regulation (FIG. 13, IV) and metabolism and biosynthesis (FIG. 13, VI). This up-regulation may not be a direct effect of P-TEFb knockdown but rather a secondary or correlated effect, to which the cell responds by overexpressing certain genes to compensate the loss of fuction of genes modulated by P-TEFb. The up-regulation of genes involved in signal transduction, transcription and cell cycle regulation (FIG. 13, I, II, and IV) suggests that these genes could complement cellular functions in P-TEFb knockdown cells or play a role in overcoming effects of down-regulated genes. An interesting correlation was observed between up-regulation and the functions of two classes of genes, i.e., transcription regulation and metabolism/biosynthesis (FIG. 13, III and VI). Translation regulator gene eIF-2, which controls a signaling pathway to activate genes involved in amino acid biosynthesis (Harding et al., Mol. Cell 6:1099-1108 (2000)), was up-regulated.

### Example 28

### Specific Silencing of TEFs in vivo

The effect of downregulating TEFs in vivo is assayed by administering siRNA targeted to one or more TEFs, e.g. Spt4, Spt5, and/or Spt6, in an animal model. The siRNA is administered using hydrodynamic transfection as previously described (McCaffrey (2002), supra; Liu (1999), supra), by intravenous injection into the tail vein (Zhang (1999), supra); or by viral delivery (Xia (2002), supra). At various time points after administration of the selected siRNA, mRNA levels for one or more TEFs, e.g., Spt4, Spt5, and/or Spt6 are measured. Additionally, the siRNA can be labeled, and the half life of the siRNA molecules is tracked using methods known in the art. Using electroporation, RNase III-prepared siRNA can be delivered into the post-implantation mouse embryos. 0.03:g-0.3 :g siRNA can efficiently silence reporter gene expression in different regions of the neural tube or other cavities of the mouse embryo (Calegari (2002), supra). Using rapid injection of the siRNA-containing physiological solution into the tail vein of postnatal mice, 0.5-5 :g siRNA can cause 36±17%-88%±3% inhibition of target gene expression. The effect of RNAi is siRNA dose-dependent and can persist for approximately 4 days after siRNA delivery (Lewis (2002), supra). By direct injection, 5-40 :g siRNA can be used to silencing target gene expression in the liver, which is central to metabolism (Lewis (2002), supra; McCaffrey (2002), supra).

### OTHER REFERENCES

Bieniasz, P. D., Grdina, T. A., Bogerd, H. P., and Cullen, B. R. (1998). Recruitment of a protein complex containing Tat and cyclin T1 to TAR governs the species specificity of HIV-1 Tat. EMBO J 17, 7056-7065.

Cullen, B. R. (1998). HIV-1 auxiliary proteins: making connections in a dying cell. Cell 93, 685-692.

Fujinaga, K., Cujec, T., Peng, J., Garriga, J., Price, D., Grana, X., and Peterlin, B. (1998). The ability of positive transcription elongation factor b to transactivate human immunodeficiency virus transcription depends on a functional kinase domain, cyclin T1 and Tat. Journal of Virology 72, 7154-7159.

Garber, M. E., Wei, P., and Jones, K. A. (1998a). HIV-1 Tat interacts with cyclin T1 to direct the P-TEFb CTD kinase complex to TAR RNA. Cold Spring Harbor Sump Quant Biol 63, 371-380.

Ivanov, D., Kwak, Y. T., Guo, J., and Gaynor, R. B. (2000). Domains in the SPT5 protein that modulate its transcriptional regulatory properties. Mol Cell Biol 20, 2970-2983.

lvanov, D., Kwak, Y. T., Nee, E., Guo, J., Garcia-Martinez, L. F., and Gaynor, R. B. (1999). Cyclin T1 domains involved in complex formation with Tat and TAR RNA are critical for tat-activation. J Mol Biol 288, 41-56.

Jones, K. A. (1997). Taking a new TAK on Tat Transactivation. Genes & Dev 11, 2593-2599.

Kim, J. B., and Sharp, P. A. (2001). Positive transcription elongation factor B phosphorylates hSPT5 and RNA polymerase II carboxyl-terninal domain independently of cyclin-dependent kinase-activating kinase. Journal of Biological Chemistry 276, 12317-12323.

Rana, T. M., and Jeang, K.-T. (1999). Biochemical and functional interactions between HIV-1 Tat protein and TAR RNA. Archives Biochem Biophysics 365, 175-185.

Renner, D. B., Yamaguchi, Y., Wada, T., Handa, H., and Price, D. H. (2001). A highly purified RNA polymerase II elongation control system. Journal of Biological Chemistry 276, 42601-42609.

Richter, S., Cao, H., and Rana, T. M. (2002a). Specific HIV-1 TAR RNA loop sequence and fuictional groups are required for human cyclinT 1-Tat-TAR ternary complex formation. Biochemistry 41, 6391-6397.

Richter, S., Ping, Y.-H., and Rana, T. M. (2002b). TAR RNA: A scaffold for the assembly of a regulatory switch in HIV replication. Proc Natl Acad Sci USA 99, 7928-7933.

Schwab, G., Duroux, I., Chavany, C., Helene, C., Saison-Behmoaras, E. (1994). An approach for new anticancer drugs: oncogene-targeted antisense DNA. Ann. Oncol. 5 Suppl. 4:55-8

Taube, R., Fujinaga, K., Wimmer, J., Barboric, M., and Peterlin, B. M. (1999). Tat transactivation: A model for the regulation of eukaryotic transcriptional elongation. Virology 264, 245-253.

Wada, T., Takagi, T., Yamaguchi, Y., Ferdous, A., Imai, T., Hirose, S., Sugimoto, S., Yano, K., Hartzog, G. A., Winston, F., et al. (1998a). DSIF, a novel transcription elongation factor that regulates RNA polymerase II processivity, is composed of human Spt4 and Spt5 homologs. Genes & Development 12, 343-356.

Wada, T., Takagi, T., Yamaguchi, Y., Watanabe, D., and Handa, H. (1998b). Evidence that P-TEFb alleviates the negative effect of DSIF on RNA polymerase II-dependent transcription in vitro. EMBO Journal 17, 7395-7403.

Wei, P., Garber, M. E., Fang, S.-M., Fischer, W. H., and Jones, K. A. (1998). A novel CDK9-associated C-type cyclin interacts directly with HIV-1 Tat and mediates its high-affinity, loop specific binding to TAR RNA. Cell 92, 451-462.

Wimmer, J., Fujinaga, K., Taube, R., Cujec, T., Zhu, Y., Peng, J., Price, D., and Peterlin, B. (1999). Interactions between Tat and TAR and human immunodeficiency virus replication are facilitated by human cyclin Ti but not cyclin T2a or T2b. Virology 255, 182-189.

Yamaguchi, Y., Takagi, T., Wada, T., Yano, K., Furuya, A., Sugimoto, S., Hasegawa, J., and Handa, H. (1999). NELF, a multisubunit complex containing RD, cooperates with DSIF to repress RNA polymerase II elongation. Cell 97, 41-51.

Zhou, Q., Chen, D., Pierstorff, E., and Luo, K. (1998). Transcription elongation factor P-TEFb mediates Tat activation of HIV-1 transcription at multiple stages. EMBO J 17, 3681-3691.

Zhang et al., Human Gene Therapy 10:1735-1737 (1999).

Liu et al., Gene Therapy 6:1258-1266 (1999).

### OTHER EMBODIMENTS

All publications, patent applications, patents, and other references mentioned herein are incorporated by reference in their entirety. In addition, the materials, methods, and examples are illustrative only and not intended to be limiting.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Those skilled in the art will recognize, or be able to ascertain using no more than routine experimentation, numerous equivalents to the specific procedures described herein. Such equivalents are considered to be within the scope of the present invention and are covered by the following claims. The appropriate components, processes, and methods of those patents, applications and other documents may be selected for the present invention and embodiments thereof.

What is claimed is:

1. An isolated ribonucleic acid (RNA) molecule comprising a first nucleotide sequence of at least 16 nucleotides sufficiently complementary to a target region of a TEF or TEF subunit mRNA sequence to direct target-specific RNA interference (RNAi), and a second nucleotide sequence of at least 16 nucleotides complementary to the first nucleotide sequence.

2. An isolated ribonucleic acid (RNA) molecule, comprising a sense strand and an antisense strand, wherein the antisense strand has a sequence sufficiently complementary to a target region of a TEF or TEF subunit mRNA sequence to direct target-specific RNA interference (RNAi) and wherein the sense strand has a sequence complementary to the sequence of the antisense strand.

3. The isolated RNA molecule of claim 2, wherein the sense and antisense strands each comprise at least 16 nucleotides.

4. The isolated RNA molecule of any one of claims 1-3, wherein the TEF mRNA is a P-TEFb mRNA

5. The isolated RNA molecule of any one of claims 1-3, wherein the TEF subunit RNA is a CycT1 mRNA.

6. The isolated RNA molecule of claim 4, wherein the CycT1 mRNA sequence is set forth as SEQ ID NO: 1.

7. The isolated RNA molecule of any one of claims 1-3, wherein the TEF subunit mRNA is a CDK9 mRNA.

8. The isolated RNA molecule of claim 6, wherein the CDK9 mRNA sequence is set forth as SEQ ID NO: 2.

9. The isolated RNA molecule of any one of claims 1-3, wherein the TEF mRNA is a DSFI mRNA.

10. The isolated RNA molecule of any one of claims 1-3, wherein the TEF subunit mRNA is a Spt5 mRNA.

11. The isolated RNA molecule of claim 10, wherein the Spt5 mRNA sequence is set forth as SEQ ID NO: 7.

12. The isolated RNAmolecule of claim 10, wherein the Spt5 mRNA sequence is set forth as SEQ ID NO: 11.

13. The isolated RNA molecule of any one of claims 1-12, wherein the first nucleotide sequence is fully complementary to the mRNA sequence.

14. The isolated RNA molecule of any one of claims 2-12, wherein the antisense strand nucleotide sequence is fully complementary to the mRNA sequence.

15. The isolated RNA molecule of any one of claims 1 and 4-13, further comprising a loop portion comprising 4-11 nucleotides that connects the two nucleotide sequences.

16. The isolated RNA molecule of any one of claims 1, 4-13 and 15, wherein the first and second nucleotide sequences each comprise 16, 17, 18 or 19 nucleotides.

17. The isolated RNA molecule of any one of claims 1, 4-13 and 15, wherein the first and second nucleotide sequences each consist of 20, 21 or 22 nucleotides.

18. The isolated RNA molecule of any one of claims 2-14, wherein the sense and antisense strands each comprise 16, 17, 18 or 19 nucleotides.

19. The isolated RNA molecule of any one of claims 2-14, wherein the sense and antisense strands each consist of 20, 21 or 22 nucleotides.

20. The isolated RNA molecule of any one of the preceeding claims, wherein the target region of the mRNA sequence is located from 100 to 300 nucleotides downstream of the start of translation of the mRNA.

21. The isolated RNA molecule of any one of the preceeding claims, wherein the target region of the mRNA sequence is located in a 5′ untranslated region (UTR) or a 3′ UTR of the mRNA.

22. The isolated RNA molecule of claim 1, wherein the first or second nucleotide sequence is selected from the group consisting of SEQ ID NO: 3, SEQ ID NO: 21, SEQ ID NO: 5 and SEQ ID NO: 23.

23. The isolated RNA molecule of claim 1, wherein the first or second nucleotide sequence is selected from the group consisting of SEQ ID NO: 8 and SEQ ID NO: 25.

24. An isolated nucleic acid molecule encoding the RNA molecule of any one of the preceding claims.

25. A vector comprising the isolated nucleic acid molecule of claim 24.

26. The vector of claim 25, which is a viral vector, retroviral vector, expression cassette, or plasmid.

27. The vector of claim 25, further comprising an RNA Polymerase III or RNA Polymerase II promoter.

28. The vector of claim 25, wherein the RNA Polymerase III promoter is the U6 or H1 promoter.

29. A host cell comprising the nucleic acid molecule of any one of claims 1-24.

30. A host cell comprising the vector of any one of claims 24-28.

31. The host cell of claim 29 or 30, which is a mammalian host cell.

32. The host cell of claim 31, which is a non-human mammalian cell.

33. The host cell of claim 31, which is a human cell.

34. A therapeutic composition comprising the nucleic acid molecule of one of claims 1-24, and a pharmaceutically acceptable carrier.

35. A method of treating a subject having a disorder characterized by aberrant or unwanted cellular proliferation, the method comprising administering to the subject a therapeutically effective amount of the composition of claim 34.

36. A method of treating a subject having a disorder characterized by aberrant or unwanted cellular proliferation, the method comprising administering to the subject a therapeutically effective amount of the nucleic acid molecule of any one of claims 1-24.

37. The method of claim 35 or 36, wherein the therapeutically effective amount is an amount effective to inhibit the expression or activity of a TEF, or subunit thereof.

38. The method of claim 37, wherein the TEF is P-TEFb

39. The method of claim 37, wherein the TEF is DSIF.

40. The method of claim 37, wherein the TEF subunit is selected from the group consisting of CycT1, CDK9 and Spt5.

41. The method of any one of claims 35-40, wherein the disorder is cancer.

42. The method of claim 41, wherein the cancer is selected from the groups consisting of carcinomas, sarcomas, metastatic disorders and hematopoietic neoplastic disorders.

43. The method of claim 42, wherein the hematopoietic neoplastic disorder is a leukemia.

44. A method of treating a subject infected with HIV, the method comprising administering to the subject a therapeutically effective amount of the composition of claim 34.

45. A method of treating a subject infected with HIV, the method comprising administering to the subject a therapeutically effective amount of the nucleic acid molecule of any one of claims 1-24.

46. The method of claim 44 or 45, wherein the therapeutically effective amount is an amount effective to inhibit the expression or activity of a TEF, or subunit thereof.

47. The method of claim 46, wherein the TEF is P-TEFb

48. The method of claim 46, wherein the TEF is DSIF.

49. The method of claim 46, wherein the TEF subunit is selected from the group consisting of CycT1, CDK9 and Spt5.

50. A method of treating a subject having a disorder characterized by aberrant or unwanted expression of a gene whose expression is regulated by P-TEFb, the method comprising administering to the subject a therapeutically effective amount of the composition of claim 34.

51. A method of treating a subject having a disorder characterized by aberrant expression of a gene whose expression is regulated by P-TEFb, the method comprising administering to the subject a therapeutically effective amount of the nucleic acid molecules of any one of claims 24.

52. The method of any one of claims 44-51, wherein the therapeutically effective amount is an amount effective to inhibit expression or activity of P-TEFb.

53. The method of claim 50 or 51, wherein the gene is selected from the list of genes in Table 1.

54. A method of treating a subject having a disorder characterized by aberrant expression of P-TEFb, the method comprising administering to the subject a therapeutically effective amount of the composition of claim 54.

55. A method of treating a subject having a disorder characterized by aberrant expression of P-TEFb, the method comprising administering to the subject a therapeutically effective amount of the nucleic acid molecules of any one of claims 1-24.

56. The method of claim 54 or 55, wherein the therapeutically effective amount is an amount effective to inhibit expression or activity of P-TEFb.

57. The method of any one of claims 50-56, wherein the disorder is HIV/AIDS.

58. The method of any one of claims 50-56, wherein the disorder is cancer.

59. The method of claim 58, wherein the cancer is selected from the group consisting of carcinomas, sarcomas, metastatic disorders and hematopoietic neoplastic disorders.

60. The method of claim 59 wherein the hematopoietic neoplastic disorder is a leukemia.

61. A method for inhibiting unwanted cellular proliferation in a subject, the method comprising administering an effective amount of an inhibitor of CDK9.

62. A method for inhibiting viral replication in a subject, comprising administering an effective amount of an inhibitor of CDK9.

63. The method of claim 61 or 62, wherein the inhibitor of CDK9 reduces CDK9 expression.

64. The method of claim 63, wherein the inhibitor is an antisense strand or a siRNA that is targeted to bind to a nucleic acid that encodes CDK9.

65. The method of claim 61 or 62, wherein the inhibitor of CDK9 reduces P-TEFb activity.

66. The method of claim 65, wherein the inhibitor is a small molecule, a dominant negative form of CDK9 or CycT1.

67. The method of claim 61, wherein the unwanted cellular proliferation is cancer.

68. The method of claim 67, wherein the cancer is selected from the group consisting of carcinomas, sarcomas, metastatic disorders and hematopoietic neoplastic disorders.

69. The method of claim 68, wherein the hematopoietic neoplastic disorder is a leukemia.

70. A method of treating a subject having a disorder characterized by aberrant expression of a gene whose expression is regulated by DSIF or Spt5, the method comprising administering to the subject a therapeutically effective amount of the composition of claim 34.

71. A method of treating a subject having a disorder characterized by aberrant expression of a gene whose expression is regulated by DSIF or Spt5, the method comprising administering to the subject a therapeutically effective amount of the nucleic acid molecules of any one of claims 1-24.

72. The method of claim 71 or 72, wherein the gene is selected from the list of genes in Table 1.

73. A method of treating a subject having a disorder characterized by aberrant expression of DSIF or Spt5, the method comprising administering to the subject a therapeutically effective amount of the composition of claim 34.

74. A method of treating a subject having a disorder characterized by aberrant expression of DSIF or Spt5, the method comprising administering to the subject a therapeutically effective amount of the nucleic acid molecule of one of claims 1-24.

75. The method of any one of claims 70-74, wherein the disorder is HIV/AIDS.

76. The method of any one of claims 70-74, wherein the disorder is cancer.

77. The method of claim 76 wherein the cancer is selected from the group consisting of carcinomas, sarcomas, metastatic disorders and hematopoietic neoplastic disorders.

78. The method of claim 77 wherein the hematopoietic neoplastic disorder is a leukemia.

79. A method for inhibiting unwanted cellular proliferation in a subject, comprising administering an effective amount of an inhibitor of Spt5.

80. A method for inhibiting viral replication in a subject, comprising administering an effective amount of an inhibitor of Spt5.

81. The method of claim 79 or 80, wherein the inhibitor of Spt5 reduces Spt5 expression.

82. The method of claim 81, wherein the inhibitor is antisense or a siRNA.

83. The method of claim 79 or 80, wherein the inhibitor of Spt5 reduces DSIF activity.

84. The method of claim 83, wherein the inhibitor is a small molecule, a dominant negative form of Spt5.

85. The method of claim 79 wherein the unwanted cellular proliferation is cancer.

86. The method of claim 85 wherein the cancer is selected from the group consisting of carcinomas, sarcomas, metastatic disorders and hematopoietic neoplastic disorders.

87. The method of claim 86 wherein the hematopoietic neoplastic disorder is a leukemia.
